(19) United States
(12) Patent Application Publication
(10) Pub. No.: US 2006/0135517 A1
(43) Pub. Date: Jun. 22, 2006

(54) Imidazolopyridines and methods of making and using the same

(76) Inventors: Wen-Cherng Lee, Lexington, MA (US); Mary Beth Carter, Arlington, MA (US); Lihong Sun, Arlington, MA (US); Claudio Chuaqui, Somerville, MA (US); Juswinder Singh, Ashland, MA (US); Paula Boriack-Sjodin, Waltham, MA (US); Michael J. Choi, Cambridge, MA (US)

Correspondence Address: Miller Canfield Paddock & Stone;Jonathan P O'Brien, 444 West Michigan Avenue, Kalamazoo, MI, 49007, US

(21) Appl. No.: 10/526,653

(22) PCT Filed: Sep. 5, 2003
(86) PCT No.: PCT/US03/27721

§ 371 Date: Nov. 1, 2005

Related U.S. Application Data
(60) Provisional application No. 60/408,812, filed on Sep. 6, 2002.

(51) Int. Cl. A61K 31/5377; C07D 471/02; A61K 31/506

(52) U.S. Cl. 514/234.5; 514/303; 514/275; 544/127; 544/331

(57) ABSTRACT

Compounds of formula I possess unexpectedly high affinity for Alk 5 and/or Alk 4, and can be useful as antagonists thereof for preventing and/or treating numerous diseases, including fibrotic disorders. In one embodiment, the invention features a compound of formula I:

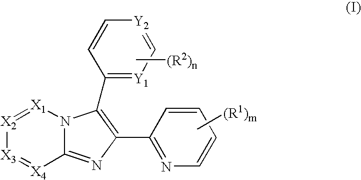

This non-provisional application is a continuation application of PCT/US2003/027721, filed on Sep. 5, 2003, which is a continuation-in-part and claims benefit of priority of U.S. Provisional application 60/408,812, filed Sep. 6, 2002. The entire disclosure of each of the aforementioned patent applications are incorporated herein by reference.

### BACKGROUND OF THE INVENTION

TGFβ (Transforming Growth Factor β) is a member of a large family of dimeric polypeptide growth factors that includes activins, inhibins, bone morphogenetic proteins (BMPs), growth and differentiation factors (GDFs) and mullerian inhibiting substance (MIS). TGFβ exists in three isoforms (TGFβ1, TGFβ2, and TGFβ3) and is present in most cells, along with its receptors. Each isoform is expressed in both a tissue-specific and developmentally regulated fashion. Each TGFβ isoform is synthesized as a precursor protein that is cleaved intracellularly into a C-terminal region (latency associated peptide (LAP)) and an N-terminal region known as mature or active TGFβ. LAP is typically non-covalently associated with mature TGFβ prior to secretion from the cell. The LAP-TGFβ complex cannot bind to the TGFβ receptors and is not biologically active. TGFβ is generally released (and activated) from the complex by a variety of mechanisms including interaction with thrombospondin-1 or plasmin.

Following activation, TGFβ binds at high affinity to the type II receptor (TGFβRII), a constitutively active serine/threonine kinase. The ligand-bound type II receptor phosphorylates the TGFβ type I receptor (Alk 5) in a glycine/serine rich domain, which allows the type I receptor to recruit and phosphorylate downstream signaling molecules, Smad2 or Smad3. See, e.g., Huse, M. et al., Mol. Cell. 8: 671-682 (2001). Phosphorylated Smad2 or Smad3 can then complex with Smad4, and the entire hetero-Smad complex translocates to the nucleus and regulates transcription of various TGFβ-responsive genes. See, e.g., Massagué, J. Ann. Rev. Biochem. Med. 67: 773 (1998).

Activins are also members of the TGFβ superfamily that are distinct from TGFβ in that they are homo- or heterodimers of activin βa or βb. Activins signal in a similar manner to TGFβ that is, by binding to a constitutive serine-threonine receptor kinase, activin type II receptor (ActRIIB), and activating a type I serine-threonine receptor, Alk 4, to phosphorylate Smad2 or Smad3. The consequent formation of a hetero-Smad complex with Smad4 also results in the activin-induced regulation of gene transcription.

Indeed, TGFβ and related factors such as activin regulate a large array of cellular processes, e.g., cell cycle arrest in epithelial and hematopoietic cells, control of mesenchymal cell proliferation and differentiation, inflammatory cell recruitment, immunosuppression, wound healing, and extracellular matrix production. See, e.g., Massagué, J. Ann. Rev. Cell. Biol. 6: 594-641 (1990); Roberts, A. B. and Sporn M. B. Peptide Growth Factors and Their Receptors, 95: 419-472 Berlin: Springer-Verlag (1990); Roberts, A. B. and Sporn M. B. Growth Factors 8:1-9 (1993); and Alexandrow, M. G., Moses, H. L. Cancer Res. 55: 1452-1457 (1995). Hyperactivity of TGFβ signaling pathway underlies many human disorders (e.g., excess deposition of extracellular matrix, an abnormally high level of inflammatory responses, fibrotic disorders, and progressive cancers). Similarly, activin signaling and overexpression of activin is linked to pathological disorders that involve extracellular matrix accumulation and fibrosis (see, e.g., Matsuse, T. et al., Am. J. Respir. Cell Mol. Biol. 13: 17-24 (1995); Inoue, S. et al., Biochem. Biophys. Res. Comm. 205: 441-448 (1994); Matsuse, T. et al, Am. J. Pathol. 148: 707-713 (1996); De Bleser et al., Hepatology 26: 905-912 (1997); Pawlowski, J. E., et al., J. Clin. Invest. 100: 639-648 (1997); Sugiyama, M. et al., Gastroenterology 114: 550-558 (1998); Munz, B. et al., EMBO J. 18: 5205-5215 (1999)) and inflammatory responses (see, e.g., Rosendahl, A. et al., Am. J. Repir. Cell Mol. Biol. 25: 60-68 (2001)). Studies have shown that TGFβ and activin can act synergistically to induce extracellular matrix (see, e.g., Sugiyama, M. et al., Gastroenterology 114: 550-558, (1998)). It is therefore desirable to develop modulators (e.g., antagonists) to signaling pathway components of the TGFβ family to prevent/treat disorders related to the malfunctioning of this signaling pathway.

### SUMMARY OF THEE INVENTION

Compounds of formula (I) are unexpectedly potent antagonists of the TGFβ family type I receptors, Alk5 and/or Alk 4. Thus, compounds of formula (I) can be employed in the prevention and/or treatment of diseases such as fibrosis (e.g., renal fibrosis, pulmonary fibrosis, and hepatic fibrosis), progressive cancers, or other diseases for which reduction of TGFβ family signaling activity is desirable.

In one aspect, the invention features a compound of formula I:

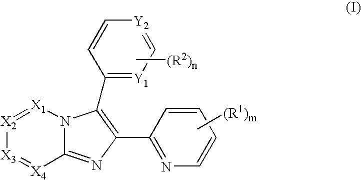

Each of X₁, X₂, X₃, and X₄ is independently CR^{x} or N; provided that only two of X₁, X₂, X₃, and X₄ can be N simultaneously. Each of Y₁ and Y₂ is independently CR^{y} or N; provided that at least one of Y₁ and Y₂ must be N. Each R¹ is independently alkyl, alkenyl, alkynyl, alkoxy, acyl, halo, hydroxy, amino, nitro, cyano, guanadino, amidino, carboxy, sulfo, mercapto, alkylsulfanyl, alkylsulfinyl, alkylsulfonyl, aminocarbonyl, alkylcarbonylamino, alkylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, heterocycloalkyl, heterocycloalkyloxy, heterocycloalkylsulfanyl, aryl, aryloxy, arylsulfanyl, aroyl, heteroaryl, heteroaryloxy, heteroarylsulfanyl, or heteroaroyl. Each R² is independently alkyl, alkenyl, alkynyl, acyl, halo, hydroxy, —NH₂, —NH(alkyl), —N(alkyl)₂, —NH(cycloalkyl), —N(alkyl)(cyclocalkyl),

—NH(heterocycloalkyl), —NH(heteroaryl), —NH-alkyl-heterocycloalkyl, —NH-alkyl-heteroaryl, —NH(aralkyl), cycloalkyl, (cycloalkyl)alkyl, aryl, aralkyl, aroyl, heterocycloalkyl, (heterocycloalkyl)alkyl, heteroaryl, heteroaralkyl, heteroaroyl, nitro, cyano, guanadino, amidino, carboxy, sulfo, mercapto, alkoxy, cycloalkyloxy, (cycloalkyl)alkoxy, aryloxy, arylalkoxy, heterocycloalkyloxy, (heterocycloalkyl)alkoxy, heteroaryloxy, heteroarylalkoxy, alkylsulfanyl, cycloalkylsulfanyl, (cycloalkyl)alkylsulfanyl, arylsulfanyl, aralkylsulfanyl, heterocycloalkylsulfanyl, (heterocycloalkyl)alkylsulfanyl, heteroarylsulfanyl, heteroarylalkylsulfanyl, alkylsulfinyl, alkylsulfonyl, aminocarbonyl, aminosulfonyl, alkylcarbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, heteroaralkylcarbonylamino, alkoxycarbonylaminoalkylamino, (heteroaryl)arylcarbonylaminoalkylamino, heteroaralkylcarbonylaminoalkylamino, (heteroaryl)arylsulfonylaminoalkylcarbonylaminoalkylamino, arylsulfonylaminoalkylamino, alkoxycarbonyl, alkylcarbonyloxy, urea, thiourea, sulfamoyl, sulfamide, or carbamoyl. m is 0, 1, 2, 3, or 4; provided that when m≧2, two adjacent R¹ groups can join together to form a 4- to 8-membered optionally substituted cyclic moiety. n is 0, 1, 2, or 3; provided that when n≧2, two adjacent R² groups can join together to form a 4- to 8-membered optionally substituted cyclic moiety. Each of R^{x} and R^{y} is independently hydrogen, alkyl, alkenyl, alkynyl, alkoxy, acyl, halo, hydroxy, amino, nitro, cyano, guanadino, amidino, carboxy, sulfo, mercapto, alkylsulfanyl, alkylsulfinyl, alkylsulfonyl, cycloalkylcarbonyl, (cycloalkyl)alkylcarbonyl, aroyl, aralkylcarbonyl, heterocycloalkylcarbonyl, (heterocycloalkyl)acyl, heteroaroyl, (heteroaryl)acyl, aminocarbonyl, alkylcarbonylamino, (amino)aminocarbonyl, alkylsulfonylaminocarbonyl, alkylsulfonylamino, cycloalkylcarbonylamino, cycloalkylsulfonylamino, (cycloalkyl)alkylcarbonylamino, (cycloalkyl)alkylsulfonylamino, arylcarbonylamino, arylsulfonylamino, aralkylcarbonylamino, aralkylsulfonylamino, (heterocycloalkyl)carbonylamino, (heterocycloalkyl)sulfonylamino, (heterocycloalkyl)alkylcarbonylamino, (heterocycloalkyl)alkylsulfonylamino, heteroarylcarbonylamino, heteroarylsulfonylamino, heteroaralkylcarbonylamino, heteroaralkylsulfonylamino, (heteroaryl)arylcarbonylaminoalkylamino, heteroaralkylcarbonylaminoalkylamino, (heteroaryl)arylsulfonylaminoalkylcarbonylaminoalkylamino, arylsulfonylaminoalkylamino, alkoxycarbonyl, alkylcarbonyloxy, urea, thiourea, sulfamoyl, sulfamide, carbamoyl, cycloalkyl, cycloalkyloxy, cycloalkylsulfanyl, (cycloalkyl)alkyl, (cycloalkyl)alkoxy, (cycloalkyl)alkylsulfanyl, heterocycloalkyl, heterocycloalkyloxy, heterocycloalkylsulfanyl, (heterocycloalkyl)alkyl, (heterocycloalkyl)alkoxy, (heterocycloalkyl)alkylsulfanyl, aryl, aryloxy, arylsulfanyl, aralkyl, aralkyloxy, aralkylsulfanyl, arylalkenyl, arylalkynyl, heteroaryl, heteroaryloxy, heteroarylsulfanyl, heteroaralkyl, (heteroaryl)alkoxy, or (heteroaryl)alkylsulfanyl.

As defined above, when m≧2, two adjacent R¹ groups can join together to form a 4- to 8-membered optionally substituted cyclic moiety. That is, the 2-pyridyl ring can fuse with a 4- to 8-membered cyclic moiety to form a moiety such as 7H-[1]pyrindinyl, 6,7-dihydro-5H-[1]pyrindinyl, 5,6,7,8-tetrahydro-quinolinyl, 5,7-dihydro-furo[3,4-b]pyridinyl, or 3,4-dihydro-1H-thiopyrano[4,3-c]pyridinyl. The fused ring moiety can be optionally substituted with one or more substituents such as alkyl (including carboxyalkyl, hydroxyalkyl, and haloalkyl such as trifluoromethyl; see definiton of “alkyl” below), alkenyl, alkynyl, cycloalkyl, heterocycloalkyl, alkoxy, aryl, heteroaryl, aryloxy, heteroaryloxy, aralkyloxy, heteroarylalkoxy, aroyl, heteroaroyl, amino, nitro, carboxy, alkoxycarbonyl, alkylcarbonyloxy, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

Similarly, when n≧2, two adjacent R² groups can join together to form a 4- to 8-membered optionally substituted cyclic moiety, thereby forming a ring fused with the pyridyl or pyrimidinyl group. Some examples of such a moiety are shown below: 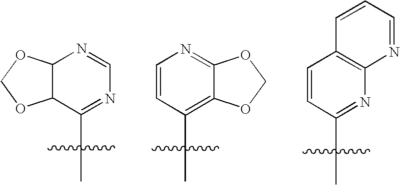
The 4- to 8-membered cyclic moiety formed by two adjacent R² groups can be optionally substituted with substituents such as alkyl (including carboxyalkyl, hydroxyalkyl, and haloalkyl such as trifluoromethyl; see definiton of “alkyl” below), alkenyl, alkynyl, cycloalkyl, heterocycloalkyl, alkoxy, aryl, heteroaryl, aryloxy, heteroaryloxy, aralkyloxy, heteroarylalkoxy, aroyl, heteroaroyl, amino, nitro, carboxy, alkoxycarbonyl, alkylcarbonyloxy, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

In one embodiment, each of X₁, X₂, and X₃ is CR^{x}. In one embodiment, each of X₂, X₃, and X₄ is independently —CH—, —C(CH₃)—, —C(OH)—, —C(NH₂)—, —C(CO—NH₂)—, —C(CO—NHOH)—, —C(NH(unsubstituted alkyl))-, —C(NH(aryl))-, —C(NH(aralkyl))-, —C(NH(heteroaryl))-,

—C(NH(heteroarylalkyl))-, —C(NH—CO-(unsubstituted alkyl))-, —C(NH—CO-(aryl))-, —C(NH—CO-(heteroaryl))-, —C(NH—CO-(aralkyl))-, —C(NH—CO-(heteroarylalkyl))-, —C(NH—SO₂-(unsubstituted alkyl))-, —C(NH—SO₂-(aryl))-, —C(NH—SO₂-(heteroaryl))-, —C(NH—SO₂-(aralkyl))-, —C(NH—SO₂-(heteroarylalkyl))-, —CNH—SO₂—NH(unsubstituted alkyl))-, —C(NH—SO₂—NH(aryl))-, —C(NH—SO₂—NH(heteroaryl))-, —C(NH—SO₂—NH(aralkyl))-, —C(NH—SO₂—NH(heteroarylalkyl))-, —C(hydroxyalkyl)-, or —C(carboxy)-, and X₁ is —CH—.

In one embodiment, each of X₁ and X₂ is —CH—; X₄ is N; and X₃ is —C(NH₂)—, —C(NH(unsubstituted alkyl))-, —C(NH(aryl))-, —C(NH(aralkyl))-, —C(NH(heteroaryl))-, —C(NH(heteroarylalkyl))-, —C(NH—CO-(unsubstituted alkyl))-, —C(NH—CO-(aryl))-, —C(NH—CO-(heteroaryl))-, —C(NH—CO-(aralkyl))-, —C(NH—CO-(heteroarylalkyl))-, —C(H—SO₂-(unsubstituted alkyl))-, —C(NH—SO₂-(aryl))-, —C(NH—SO₂-(heteroaryl))-, —C(NH—SO₂-(aralkyl))-, —C(NH—SO₂-(heteroarylalkyl))-, —C(NH—SO₂—NH(unsubstituted alkyl))-, —CH—SO₂—NH(aryl))-,

—C(NH—SO₂—NH(heteroaryl))-, —C(NH—SO₂—NH(aralkyl))-, or —C(NH—SO₂—NH(heteroarylalkyl))-.

In one embodiment, both Y₁ are Y₂ are N.

In one embodiment, m is 0, 1, or 2 (e.g., m is 1). In one embodiment, R¹ is substituted at the 5-position or the 6-position (i.e., R¹ can be mono-substituted at either the 5-position or the 6-position or R¹ can be di-substituted at both the 5- and the 6-position). In one embodiment, R¹ is C₁₋₄ alkyl, C₁₋₄ alkoxy, C₁₋₄ alkylthio, halo, amino, aminocarbonyl, or alkoxycarbonyl.

In one embodiment, n is 1 or 2 (e.g., n is 1).

In one embodiment, each R¹ is independently unsubstituted alkyl (e.g., 6-methyl, 6-ethyl, 6-n-propyl, or 6-isopropyl), hydroxyalkyl, haloalkyl (e.g., 6-trifluoromethyl), aminoalkyl, aryloxyalkyl, heteroaralkyloxyalkyl, unsubstituted alkenyl (e.g., 6-vinyl), alkoxy, acyl, halo, hydroxy, carboxy, cyano, guanadino, amidino, amino (e.g., —NH₂, monoalkylamino, dialkylamino, monoheterocycloalkylamino, monoheteroarylamino, mono(heterocyclylalkyl)amino, mono(aralkyl)amino, or mono(heteroaralkyl)amino), carboxy, mercapto, alkylsulfanyl, alkylsulfinyl, alkylsulfonyl, aminocarbonyl (e.g., —CONH₂, —CONH(alkyl), or —CO—N(alkyl)₂), alkylcarbonylamino (e.g., —NH—CO-alkyl or —N(alkyl)-CO-alkyl), alkoxycarbonyl, alkylcarbonyloxy, alkylsulfonyl, sulfamoyl (e.g., —SO₂—NH₂, —SO₂—NH(alkyl), or —SO₂—N(alkyl)₂), cycloalkyl (e.g., 6-cyclopropyl), heterocycloalkyl, (heterocycloalkyl)alkyl, heteroaryl, or heteroaralkyl.

In one embodiment, each R² is independently unsubstituted alkyl, hydroxyalkyl, haloalkyl, aminoalkyl (e.g., aminomethyl), aryloxyalkyl, heteroaralkyloxyalkyl, alkoxy, acyl, halo, hydroxy, carboxy, cyano, guanadino, amidino, —NH₂, monoalkylamino, dialkylamino, monocycloalkylamino, monoheterocycloalkylamino (e.g., —NH-piperidinyl or —NH-morpholino), monoheteroarylamino (e.g., —NH-tetrazolyl, —NH-pyrazolyl, or —NH-imidazolyl), mono((heterocycloalkyl)alkyl)amino (e.g., —NH—(CH₂)₁₋₃-piperidinyl or —NH—(CH₂)₁₋₃-morpholino), mono(heteroaralkyl)amino (e.g., —NH—(CH₂)₁₋₃-tetrazolyl, —NH—(CH₂)₁₋₃-pyrazolyl, or —NH—(CH₂)₁₋₃-imidazolyl), —N(alkyl)(cycloalkyl), mercapto, alkylsulfanyl, alkylsulfinyl, alkylsulfonyl, —CONH₂, —CONH(alkyl), —CO—N(alkyl)₂, —NH—CO-alkyl,

—N(alkyl)-CO-alkyl, —CO₂-alkyl, —O—CO-alkyl, —SO₂—NH₂, —SO₂—NH(alkyl), —SO₂—N(alkyl)₂,

—NH—SO₂-alkyl, —N(alkyl)-SO₂-alkyl, —NH—CO—NH(alkyl), —N(alkyl)-CO—NH(alkyl), —NH—SO₂—NH(alkyl), —N(alkyl)-SO₂—NH(alkyl), heterocycloalkyl, or heteroaryl (e.g., imidazolyl, pyrazolyl, tetrazolyl, or pyridyl). For example, R² is substituted at the 3-position and is guanadino, amidino, —NH₂, monoalkylamino, dialkylamino, monocycloalkylamino, monoheterocycloalkylamino, monoheteroarylamino, mono((heterocycloalkyl)alkyl)amino, mono(heteroaralkyl)amino, —NH—CO—NH(alkyl), —N(alkyl)-CO—NH(alkyl), —NH—SO₂—NH(alkyl), —N(alkyl)-SO₂—NH(alkyl), heterocycloalkyl, or heteroaryl.

In one embodiment, each R^{x} is independently hydrogen, unsubstituted alkyl, hydroxyalkyl (e.g., hydroxy-C₁₋₄ alkyl such as hydroxyethyl), haloalkyl (e.g., trifluoromethyl), aminoalkyl, aryloxyalkyl, heteroaralkyloxyalkyl, alkoxy (e.g., C₁₋₄ alkoxy such as methoxy or C₁₋₄ haloalkoxy such as —OCF₃), halo (e.g., chloro or bromo), hydroxy, carboxy, cyano, guanadino, amidino, amino (e.g., —NH₂, —NH(alkyl), —N(alkyl)₂,

—NH(heterocycloalkyl), —NH(heteroaryl), —NH(heterocycloalkyl-alkyl), —NH(aralkyl), or

—NH(heteroaralkyl)), carboxy, (heteroaryl)acyl, aminocarbonyl (e.g., —CO—NH₂, —CO—NH—(CH₂)₀₋₃—COOH, —CO—NH—(CH₂)₀₋₃—OH, —CO—NH—(CH₂)₀₋₃-heteroaryl (e.g., —CO—NH—(CH₂)₀₋₃-tetrazolyl, —CO—NH—(CH₂)₀₋₃-pyrazolyl, or —CO—NH—(CH₂)₀₋₃-imidazolyl), —CO—NH—(CH₂)₀₋₃-heterocycloalkyl (e.g., —CO—NH—(CH₂)₀₋₃-piperidinyl or —CO—NH—(CH₂)₀₋₃-morpholino), or —CO—NH—(CH₂)₀₋₃-aryl (e.g., —CO—NH—(CH₂)₀₋₃-phenyl), heteroarylcarbonylamino, (heterocycloalkyl)alkoxy, (heteroaryl)alkoxy, (heteroaryl)alkylsulfanyl, heterocycloalkyl (e.g., morpholino, pyrazinyl, or piperidinyl), (heterocycloalkyl)alkyl (e.g., morpholino-C₁₋₄ alkyl, pyrazinyl-C₁₋₄ alkyl, or piperidinyl-C₁₋₄ alkyl), heteroaryl (e.g., imidazolyl, pyrazolyl, tetrazolyl, or pyridyl), or heteroaralkyl (e.g., imidazolyl-C₁₋₄ alkyl, pyrazolyl-C₁₋₄ alkyl, tetrazolyl-C₁₋₄ alkyl, or pyridyl-C₁₋₄ alkyl). Some examples of —NH(alkyl) are

—NH(haloalkyl) (e.g., —NHCF₃), —NH(carboxyalkyl) (e.g., —NH(CH₂)₁₋₃COOH), and —NH(hydroxyalkyl) (e.g., —NH(CH₂)₁₋₃OH). Some examples of —NH(heteroaryl) are —NH(tetrazolyl), —NH(pyrazolyl), and —NH(imidazolyl). Some examples of —NH(heterocycloalkylalkyl) are —NH(piperazinylalkyl) (e.g., —NH(CH₂)₁₋₃-piperizine) and

—NH(morpholino-alkyl) (e.g., —NH(CH₂)₁₋₃-morpholine). Some examples of —NH(heteroaralkyl) are —NH(tetrazolylalkyl) (e.g., —NH(CH₂)₀₋₃-tetrazole), —NH(pyrazolyl-alkyl) (e.g., —NH(CH₂)₀₋₃-pyrazole), and —NH(imidazolyl-alkyl) (e.g., —NH(CH₂)₀₋₃-imidazole).

In one embodiment, R^{y} is hydrogen, unsubstituted alkyl, hydroxyalkyl, haloalkyl (e.g., trifluoromethyl), aminoalkyl, aryloxyalkyl, heteroaralkyloxyalkyl, alkoxy, halo, hydroxy, carboxy, cyano, guanadino, amidino, amino (e.g., —NH₂, —NH(alkyl), —N(alkyl)₂, —NH(cycloalkyl),

—NH(heterocycloalkyl), —NH(heteroaryl), —NH(heterocycloalkyl-alkyl), —NH(aralkyl), or

—NH(heteroaralkyl)), carboxy, (heteroaryl)acyl, aminocarbonyl, heteroarylcarbonylamino, (heterocycloalkyl)alkoxy, (heteroaryl)alkoxy, (heteroaryl)alkylsulfanyl, heterocycloalkyl, (heterocycloalkyl)alkyl, heteroaryl, or heteroaralkyl.

In one embodiment, X₁ is N. For example, X₁ is N and each of X₂, X₃, and X₄ is independently CR^{x}.

In one embodiment, X₂ is N. For example, X₂ is N and each of X₁, X₃, and X₄ is independently CR^{x}.

In one embodiment, X₃ is N. For example, X₃ is N and each of X₁, X₂, and X₄ is independently CR^{x}.

In one embodiment, X₄ is N. For example, X₄ is N and each of X₁, X₂, and X₃ is independently CR^{x}.

Some examples of a compound of formula (I) are
(2-Methoxy-ethyl)-{4-[2-(6-methyl-pyridin-2-yl)-imidazo[1,2-a]pyridin-3-yl]-pyrimidin-2-yl}-amine;
(3-{4-[2-(6-Methyl-pyridin-2-yl)-imidazo[1,2-a]pyridin-3-yl]-pyrimidin-2-ylamino}-propyl)-carbamic acid tert-butyl ester;
(3-Imidazol-1-yl-propyl)-{4-[2-(6-methyl-pyridin-2-yl)-imidazo[1,2-a]pyridin-3-yl]-pyrimidin-2-yl}-amine;
(4-Methoxy-benzyl)-{4-[2-(6-methyl-pyridin-2-yl)-imidazo[1,2-a]pyridin-3-yl]-pyrimidin-2-yl}-amine;
[2-(6-Methyl-pyridin-2-yl)-3-(2-methylsulfanyl-pyrimidin-4-yl)-imidazo[1,2-a]pyridin-6-yl]-methanol;
3-(2-Methanesulfonyl-pyrimidin-4-yl)-2-(6-methyl-pyridin-2-yl)-imidazo[1,2-a]pyridine;
(4-{4-[2-(6-Methyl-pyridin-2-yl)-imidazo[1,2-a]pyridin-3-yl]-pyrimidin-2-ylamino}-butyl)-carbamic acid tert-butyl ester;
(4-Amino-benzyl)-{4-[2-(6-methyl-pyridin-2-yl)-imidazo[1,2-a]pyridin-3-yl]-pyrimidin-2-yl}-amine;
(5-{4-[2-(6-Methyl-pyridin-2-yl)-imidazo[1,2-a]pyridin-3-yl]-pyrimidin-2-ylamino}-pentyl)-carbamic acid tert-butyl ester;
[3-(2-Amino-pyrimidin-4-yl)-2-(6-methyl-pyridin-2-yl)-imidazo[1,2-a]pyridin-6-yl]-methanol;
[3-(2-amino-pyrimidin-4-yl)-2-(6-methyl-pyridin-2-yl)-imidazo[1,2-a]pyridin-7-yl]-methanol;
[3-(2-Amino-pyrimidin-4-yl)-6-methyl-2-(6-methyl-pyridin-2-yl)-imidazo[1,2-a]pyridin-8-yl]-(2-morpholin-4-yl-ethyl)-amine;
[3-(2-Amino-pyrimidin-4-yl)-6-methyl-2-(6-methyl-pyridin-2-yl)-imidazo[1,2-a]pyridin-8-yl]-(2-pyridin-2-yl-ethyl)-amine;
[3-(2-Amino-pyrimidin-4-yl)-6-methyl-2-(6-methyl-pyridin-2-yl)-imidazo[1,2-a]pyridin-8-yl]-(2-pyridin-3-yl-ethyl)-amine;
[3-(2-methanesulfonyl-pyrimidin-4-yl)-2-(6-methyl-pyridin-2-yl)-imidazo[1,2-a]pyridin-6-yl]-methanol;
[3-(2-Amino-pyrimidin-4-yl)-6-methyl-2-(6-methyl-pyridin-2-yl)-imidazo[1,2-a]pyridin-8-yl]-(2-pyridin-4-yl-ethyl)-amine;
[3-(2-Amino-pyrimidin-4-yl)-6-methyl-2-(6-methyl-pyridin-2-yl)-imidazo[1,2-a]pyridin-8-yl]-(3-morpholin-4-yl-propyl)-amine;
[3-(4-Methyl-piperazin-1-yl)-propyl]-{4-[2-(6-methyl-pyridin-2-yl)-imidazo[1,2-a]pyridin-3-yl]-pyrimidin-2-yl}-amine;
[3-(4-Methyl-piperidin-1-yl)-propyl]-{4-[2-(6-methyl-pyridin-2-yl)-imidazo[1,2-a]pyridin-3-yl]-pyrimidin-2-yl}-amine;
[4-(2-Pyridin-2-yl-imidazo[1,2-a]pyridin-3-yl)-pyrimidin-2-yl]-pyridin-3-ylmethyl-amine;
{4-[2-(6-Methyl-pyridin-2-yl)-imidazo[1,2-a]pyridin-3-yl]-pyrimidin-2-yl}-((R)-1-phenyl-ethyl)-amine;
{4-[2-(6-Methyl-pyridin-2-yl)-imidazo[1,2-a]pyridin-3-yl]-pyrimidin-2-yl}-((S)-1-phenyl-ethyl)-amine;
{4-[2-(6-Methyl-pyridin-2-yl)-imidazo[1,2-a]pyridin-3-yl]-pyrimidin-2-yl}-(1H-tetrazol-5-yl)-amine;
{4-[2-(6-Methyl-pyridin-2-yl)-imidazo[1,2-a]pyridin-3-yl]-pyrimidin-2-yl}-(2H-pyrazol-3-yl)-amine;
{4-[2-(6-Methyl-pyridin-2-yl)-imidazo[1,2-a]pyridin-3-yl]-pyrimidin-2-yl}-(2-morpholin-4-yl-ethyl)-amine;
{4-[2-(6-Methyl-pyridin-2-yl)-imidazo[1,2-a]pyridin-3-yl]-pyrimidin-2-yl}-(2-pyridin-2-yl-ethyl)-amine;
{4-[2-(6-Methyl-pyridin-2-yl)-imidazo[1,2-a]pyridin-3-yl]-pyrimidin-2-yl}-(2-pyridin-3-yl-ethyl)-amine;
{4-[2-(6-Methyl-pyridin-2-yl)-imidazo[1,2-a]pyridin-3-yl]-pyrimidin-2-yl}-(2-pyridin-4-yl-ethyl)-amine;
{4-[2-(6-Methyl-pyridin-2-yl)-imidazo[1,2-a]pyridin-3-yl]-pyrimidin-2-yl}-(3-morpholin-4-yl-propyl)-amine;
{4-[2-(6-Methyl-pyridin-2-yl)-imidazo[1,2-a]pyridin-3-yl]-pyrimidin-2-yl}-(3-piperidin-1-yl-propyl)-amine;
{4-[2-(6-Methyl-pyridin-2-yl)-imidazo[1,2-a]pyridin-3-yl]-pyrimidin-2-yl}-[1,3,4]thiadiazol-2-yl-amine;
2-(6-Methyl-pyridin-2-yl)-3-(2-methylsulfanyl-pyrimidin-4-yl)-imidazo[1,2-a]pyridine;
2-(6-Methyl-pyridin-2-yl)-3-(2-methylsulfanyl-pyrimidin-4-yl)-imidazo[1,2-a]pyridine-6-carboxylic acid methyl ester;
2-(6-Methyl-pyridin-2-yl)-3-(2-methylsulfanyl-pyrimidin-4-yl)-imidazo[1,2-a]pyridine-7-carboxylic acid ethyl ester;
2-(6-Methyl-pyridin-2-yl)-3-(2-methylsulfanyl-pyrimidin-4-yl)-imidazo[1,2-a]pyrimidin-7-yl amine;
{7,7-Dimethyl-8-[5-(4-{4-[2-(6-methyl-pyridin-2-yl)-imidazo[1,2-a]pyridin-3-yl]-pyrimidin-2-ylamino}-butylcarbamoyl)-pentyl]-2-oxo-4-trifluoromethyl-7,8-dihydro-2H-1-oxa-8-aza-anthracen-5-yl}-methanesulfonic acid;
2-(2,7-Difluoro-6-hydroxy-3-oxo-9,9a-dihydro-3H-xanthen-9-yl)-3,5,6-trifluoro-4-[(4-{4-[2-(6-methyl-pyridin-2-yl)-imidazo[1,2-a]pyridin-3-yl]-pyrimidin-2-ylamino}-butylcarbamoyl)-methylsulfanyl]-benzoic acid;
2-(6-Methyl-pyridin-2-yl)-3-(2-morpholin-4-yl-pyrimidin-4-yl)-imidazo[1,2-a]pyridine;
2-(6-Methyl-pyridin-2-yl)-3-(2-piperidin-1-yl-pyrimidin-4-yl)-imidazo[1,2-a]pyridine;
2-(6-Methyl-pyridin-2-yl)-3-(2-pyrrolidin-1-yl-pyrimidin-4-yl)-imidazo[1,2-a]pyridine;
2-(6-Methyl-pyridin-2-yl)-3-[2-(1H-tetrazol-5-yl)-pyrimidin-4-yl]-imidazo[1,2-a]pyridine;
2-(6-Methyl-pyridin-2-yl)-3-pyrimidin-4-yl-imidazo[1,2-a]pyridine;
2-(6-Methyl-pyridin-2-yl)-3-pyrimidin-4-yl-imidazo[1,2-a]pyrimidin-7-ylamine;
3-(2-Amino-pyrimidin-4-yl)-2-(6-methyl-pyridin-2-yl)-imidazo[1,2-a]pyridin-8-ylamine;
3-(2-Amino-pyrimidin-4-yl)-2-(6-methyl-pyridin-2-yl)-imidazo[1,2-a]pyridine-6-carbonitrile;
3-(2-Amino-pyrimidin-4-yl)-2-(6-methyl-pyridin-2-yl)-imidazo[1,2-a]pyridine-6-carboxylic acid;
3-(2-Amino-pyrimidin-4-yl)-2-(6-methyl-pyridin-2-yl)-imidazo[1,2-a]pyridine-6-carboxylic acid ([1,4]dioxan-2-ylmethyl)-amide;
3-(2-Amino-pyrimidin-4-yl)-2-(6-methyl-pyridin-2-yl)-imidazo[1,2-a]pyridine-6-carboxylic acid ([1,4]dioxan-2-ylmethyl)-amide;
3-(2-Amino-pyrimidin-4-yl)-2-(6-methyl-pyridin-2-yl)-imidazo[1,2-a]pyridine-6-carboxylic acid (2-dimethylamino-ethyl)-amide;
3-(2-Amino-pyrimidin-4-yl)-2-(6-methyl-pyridin-2-yl)-imidazo[1,2-a]pyridine-6-carboxylic acid (2-methoxy-ethyl)-amide;
3-(2-Amino-pyrimidin-4-yl)-2-(6-methyl-pyridin-2-yl)-imidazo[1,2-a]pyridine-6-carboxylic acid (2-thiophen-2-yl-ethyl)-amide;
3-(2-Amino-pyrimidin-4-yl)-2-(6-methyl-pyridin-2-yl)-imidazo[1,2-a]pyridine-6-carboxylic acid [3-(4-methyl-piperazin-1-yl)-propyl]-amide;
3-(2-Amino-pyrimidin-4-yl)-2-(6-methyl-pyridin-2-yl)-imidazo[1,2-a]pyridine-6-carboxylic acid amide;
3-(2-Amino-pyrimidin-4-yl)-2-(6-methyl-pyridin-2-yl)-imidazo[1,2-a]pyridine-6-carboxylic acid cyclopropylamide;
3-(2-Amino-pyrimidin-4-yl)-2-(6-methyl-pyridin-2-yl)-imidazo[1,2-a]pyridine-6-carboxylic acid ethylamide;
3-(2-Amino-pyrimidin-4-yl)-2-(6-methyl-pyridin-2-yl)-imidazo[1,2-a]pyridine-6-carboxylic acid hydroxyamide;
3-(2-Amino-pyrimidin-4-yl)-2-(6-methyl-pyridin-2-yl)-imidazo[1,2-a]pyridine-6-carboxylic acid methoxy-amide;
3-(2-Amino-pyrimidin-4-yl)-2-(6-methyl-pyridin-2-yl)-imidazo[1,2-a]pyridine-6-carboxylic acid methyl ester;
3-(2-Amino-pyrimidin-4-yl)-2-(6-methyl-pyridin-2-yl)-imidazo[1,2-a]pyridine-7-carboxylic acid;
3-(2-Amino-pyrimidin-4-yl)-2-(6-methyl-pyridin-2-yl)-imidazo[1,2-a]pyridine-7-carboxylic acid ([1,4]dioxan-2-ylmethyl)-amide;
3-(2-Amino-pyrimidin-4-yl)-2-(6-methyl-pyridin-2-yl)-imidazo[1,2-a]pyridine-7-carboxylic acid (2-amino-ethyl)-amide;
3-(2-Amino-pyrimidin-4-yl)-2-(6-methyl-pyridin-2-yl)-imidazo[1,2-a]pyridine-7-carboxylic acid (2-dimethylamino-ethyl)-amide;
3-(2-Amino-pyrimidin-4-yl)-2-(6-methyl-pyridin-2-yl)-imidazo[1,2-a]pyridine-7-carboxylic acid (2-hydroxy-ethyl)-amide;
3-(2-Amino-pyrimidin-4-yl)-2-(6-methyl-pyridin-2-yl)-imidazo[1,2-a]pyridine-7-carboxylic acid (2-oxo-2-pyridin-3-yl-ethyl)-amide;
3-(2-Amino-pyrimidin-4-yl)-2-(6-methyl-pyridin-2-yl)-imidazo[1,2-a]pyridine-7-carboxylic acid (2-thiophen-2-yl-ethyl)-amide;
3-(2-Amino-pyrimidin-4-yl)-2-(6-methyl-pyridin-2-yl)-imidazo[1,2-a]pyridine-7-carboxylic acid (piperidin-3-ylmethyl)-amide;
3-(2-Amino-pyrimidin-4-yl)-2-(6-methyl-pyridin-2-yl)-imidazo[1,2-a]pyridine-7-carboxylic acid 2,2-dimethylhydrazide;
3-(2-Amino-pyrimidin-4-yl)-2-(6-methyl-pyridin-2-yl)-imidazo[1,2-a]pyridine-7-carboxylic acid amide;
3-(2-Amino-pyrimidin-4-yl)-2-(6-methyl-pyridin-2-yl)-imidazo[1,2-a]pyridine-7-carboxylic acid cyclopropylamide;
3-(2-Amino-pyrimidin-4-yl)-2-(6-methyl-pyridin-2-yl)-imidazo[1,2-a]pyridine-7-carboxylic acid ethyl ester;
3-(2-Amino-pyrimidin-4-yl)-2-(6-methyl-pyridin-2-yl)-imidazo[1,2-a]pyridine-7-carboxylic acid ethylamide;
3-(2-Amino-pyrimidin-4-yl)-2-(6-methyl-pyridin-2-yl)-imidazo[1,2-a]pyridine-7-carboxylic acid hydroxyamide;
3-(2-Amino-pyrimidin-4-yl)-2-(6-methyl-pyridin-2-yl)-imidazo[1,2-a]pyridine-7-carboxylic acid methoxy-amide;
3-(2-Amino-pyrimidin-4-yl)-2-(6-methyl-pyridin-2-yl)-imidazo[1,2-a]pyrimidin-7-ylamine;
3-(2-Azetidin-1-yl-pyrimidin-4-yl)-2-(6-methyl-pyridin-2-yl)-imidazo[1,2-a]pyridine;
3-(2-Methanesulfonyl-pyrimidin-4-yl)-2-(6-methyl-pyridin-2-yl)-imidazo[1,2-a]pyridine-7-carboxylic acid ethyl ester;
3-(2-Methanesulfonyl-pyrimidin-4-yl)-2-(6-methyl-pyridin-2-yl)-imidazo[1,2-a]pyridine-6-carboxylic acid methyl ester;
3-(2-Methanesulfonyl-pyrimidin-4-yl)-7-methyl-2-(6-methyl-pyridin-2-yl)-imidazo[1,2-a]pyridine;
3-(2-Methanesulfonyl-pyrimidin-4-yl)-8-methyl-2-(6-methyl-pyridin-2-yl)-imidazo[1,2-a]pyridine;
3,3-Dimethyl-N-[2-(6-methyl-pyridin-2-yl)-3-(2-methylsulfanyl-pyrimidin-4-yl)-imidazo[1,2-a]pyrimidin-7-yl]-butyramide;
3-(2-Methanesulfonyl-pyrimidin-4-yl)-2-(6-methyl-pyridin-2-yl)-imidazo[1,2-a]pyridine-6-carbonitrile;
3-(2-Methylsulfanyl-pyrimidin-4-yl)-2-pyridin-2-yl-imidazo[1,2-a]pyridine;
3,6-Dichloro-N-(4-{4-[2-(6-methyl-pyridin-2-yl)-imidazo[1,2-a]pyridin-3-yl]-pyrimidin-2-ylamino}-butyl)-2-(2,4,5,7-Tetrachloro-6-hydroxy-3-oxo-9,9a-dihydro-3H-xanthen-9-yl)-terephthalamic acid;
3-[2-(2-Methyl-aziridin-1-yl)-pyrimidin-4-yl]-2-(6-methyl-pyridin-2-yl)-imidazo[1,2-a]pyridine;
3-[2-(4-Methyl-piperazin-1-yl)-pyrimidin-4-yl]-2-(6-methyl-pyridin-2-yl)-imidazo[1,2-a]pyridine;
3-{[3-(2-Amino-pyrimidin-4-yl)-2-(6-methyl-pyridin-2-yl)-imidazo[1,2-a]pyridine-6-carbonyl]-amino}-propionic acid methyl ester;
3-{[3-(2-Amino-pyrimidin-4-yl)-2-(6-methyl-pyridin-2-yl)-imidazo[1,2-a]pyridine-7-carbonyl]-amino}-propionic acid methyl ester;
3-{4-[2-(6-Methyl-pyridin-2-yl)-imidazo[1,2-a]pyridin-3-yl]-pyrimidin-2-ylamino}-phenol;
4-(2-{4-[2-(6-Methyl-pyridin-2-yl)-imidazo[1,2-a]pyridin-3-yl]-pyrimidin-2-ylamino}-ethyl)-benzenesulfonamide;
4-(2-Pyridin-2-yl-imidazo[1,2-a]pyridin-3-yl)-pyrimidin-2-ylamine;
4-[2-(6-Chloro-pyridin-2-yl)-imidazo[1,2-a]pyridin-3-yl]-pyrimidin-2-ylamine;
4-[2-(6-Methyl-pyridin-2-yl)-7-trifluoromethyl-imidazo[1,2-a]pyridin-3-yl]-pyrimidin-2-ylamine;
4-[2-(6-Methyl-pyridin-2-yl)-imidazo[1,2-a]pyridin-3-yl]-pyrimidin-2-ylamine;
4-[2-(6-Methyl-pyridin-2-yl)-imidazo[1,2-a]pyridin-3-yl]-pyrimidine-2-carbonitrile;
4-[2-(6-Methyl-pyridin-2-yl)-imidazo[1,2-a]pyridin-3-yl]-pyrimidine-2-carboxylic acid amide;
4-[6-Bromo-2-(6-methyl-pyridin-2-yl)-imidazo[1,2-a]pyridin-3-yl]-pyrimidin-2-ylamine;
4-[6-Chloro-2-(6-methyl-pyridin-2-yl)-imidazo[1,2-a]pyridin-3-yl]-pyrimidin-2-ylamine;
4-[6-Fluoro-2-(6-methyl-pyridin-2-yl)-imidazo[1,2-a]pyridin-3-yl]-pyrimidin-2-ylamine;
4-[6-Methyl-2-(6-methyl-pyridin-2-yl)-8-(2-morpholin-4-yl-ethylamino)-imidazo[1,2-a]pyridin-3-yl]-pyrimidin-2-ol;
4-[6-Methyl-2-(6-methyl-pyridin-2-yl)-8-(2-pyridin-2-yl-ethylamino)-imidazo[1,2-a]pyridin-3-yl]-pyrimidin-2-ol;
4-[6-Methyl-2-(6-methyl-pyridin-2-yl)-8-(2-pyridin-3-yl-ethylamino)-imidazo[1,2-a]pyridin-3-yl]-pyrimidin-2-ol;
4-[6-Methyl-2-(6-methyl-pyridin-2-yl)-8-(2-pyridin-4-yl-ethylamino)-imidazo[1,2-a]pyridin-3-yl]-pyrimidin-2-ol;
4-[6-Methyl-2-(6-methyl-pyridin-2-yl)-8-morpholin-4-yl-imidazo[1,2-a]pyridin-3-yl]-pyrimidin-2-ol;
4-[6-Methyl-2-(6-methyl-pyridin-2-yl)-8-morpholin-4-yl-imidazo[1,2-a]pyridin-3-yl]-pyrimidin-2-ylamine;
4-[6-Methyl-2-(6-methyl-pyridin-2-yl)-imidazo[1,2-a]pyridin-3-yl]-pyrimidin-2-ylamine;
4-[7-Aminomethyl-2-(6-methyl-pyridin-2-yl)-imidazo[1,2-a]pyridin-3-yl]-pyrimidin-2-ylamine;
4-[7-Methyl-2-(6-methyl-pyridin-2-yl)-imidazo[1,2-a]pyridin-3-yl]-pyrimidin-2-ylamine;
4-[8-Benzyloxy-2-(6-methyl-pyridin-2-yl)-imidazo[1,2-a]pyridin-3-yl]-pyrimidin-2-ol;
4-[8-Benzyloxy-2-(6-methyl-pyridin-2-yl)-imidazo[1,2-a]pyridin-3-yl]-pyrimidin-2-ylamine;
4-[8-Bromo-6-methyl-2-(6-methyl-pyridin-2-yl)-imidazo[1,2-a]pyridin-3-yl]-pyrimidin-2-ol;
4-[8-Methyl-2-(6-methyl-pyridin-2-yl)-imidazo[1,2-a]pyridin-3-yl]-pyrimidin-2-ylamine;
6-Chloro-3-(2-methanesulfonyl-pyrimidin-4-yl)-2-(6-methyl-pyridin-2-yl)-imidazo[1,2-a]pyridine;
5-Dimethylamino-naphthalene-1-sulfonic acid (4-{4-[2-(6-methyl-pyridin-2-yl)-imidazo[1,2-a]pyridin-3-yl]-pyrimidin-2-ylamino}-butyl)-amide;
6-(2,7-Difluoro-6-hydroxy-3-oxo-3H-xanthen-9-yl)-N-(4-{4-[2-(6-methyl-pyridin-2-yl)-imidazo[1,2-a]pyridin-3-yl]-pyrimidin-2-ylamino}-butyl)-isophthalamic acid;
6-Amino-9-[2-carboxy-5-(4-{4-[2-(6-methyl-pyridin-2-yl)-imidazo[1,2-a]pyridin-3-yl]-pyrimidin-2-ylamino}-butylcarbamoyl)-phenyl]-xanthen-3-ylidene-ammonium;
6-Bromo-2-(6-methyl-pyridin-2-yl)-3-(2-methylsulfanyl-pyrimidin-4-yl)-imidazo[1,2-a]pyridine;
6-Fluoro-2-(6-methyl-pyridin-2-yl)-3-(2-methylsulfanyl-pyrimidin-4-yl)-imidazo[1,2-a]pyridine;
7-Amino-4-methyl-3-[(4-{4-[2-(6-methyl-pyridin-2-yl)-imidazo[1,2-a]pyridin-3-yl]-pyrimidin-2-ylamino}-butylcarbamoyl)-methyl]-2-oxo-2H-chromene-6-sulfonic acid;
Cyclobutyl-{4-[2-(6-methyl-pyridin-2-yl)-imidazo[1,2-a]pyridin-3-yl]-pyrimidin-2-yl)-amine;
Cyclopentyl-{4-[2-(6-methyl-pyridin-2-yl)-imidazo[1,2-a]pyridin-3-yl]-pyrimidin-2-yl}-amine;
Cyclopropyl-{4-[2-(6-methyl-pyridin-2-yl)-imidazo[1,2-a]pyridin-3-yl]-pyrimidin-2-yl}-amine;
Cyclopropyl-methyl-{4-[2-(6-methyl-pyridin-2-yl)-imidazo[1,2-a]pyridin-3-yl]-pyrimidin-2-yl}-amine;
Dimethyl-{4-[2-(6-methyl-pyridin-2-yl)-imidazo[1,2-a]pyridin-3-yl]-pyrimidin-2-yl}-amine;
Isopropyl-{4-[2-(6-methyl-pyridin-2-yl)-imidazo[1,2-a]pyridin-3-yl]-pyrimidin-2-yl}-amine;
Methyl-{4-[2-(6-methyl-pyridin-2-yl)-imidazo[1,2-a]pyridin-3-yl]-pyrimidin-2-yl}-amine;
N-(2-{4-[2-(6-Methyl-pyridin-2-yl)-imidazo[1,2-a]pyridin-3-yl]-pyrimidin-2-ylamino}-ethyl)-acetamide;
N-(4-{4-[2-(6-Methyl-pyridin-2-yl)-imidazo[1,2-a]pyridin-3-yl]-pyrimidin-2-ylamino}-butyl)-acetamide;
N,N-Dimethyl-N′-{4-[2-(6-methyl-pyridin-2-yl)-imidazo[1,2-a]pyridin-3-yl]-pyrimidin-2-yl}-ethane-1,2-diamine;
N-[2-(6-Methyl-pyridin-2-yl)-3-(2-methylsulfanyl-pyrimidin-4-yl)-imidazo[1,2-a]pyrimidin-7-yl]-3-pyridin-3-yl-propionamide;
N-[2-(6-Methyl-pyridin-2-yl)-3-(2-methylsulfanyl-pyrimidin-4-yl)-imidazo[1,2-a]pyrimidin-7-yl]-nicotinamide;
N-[2-(6-Methyl-pyridin-2-yl)-3-(2-methylsulfanyl-pyrimidin-4-yl)-imidazo[1,2-a]pyrimidin-7-yl]-propionamide;
N-[3-(2-Amino-pyrimidin-4-yl)-2-(6-methyl-pyridin-2-yl)-imidazo[1,2-a]pyridine-6-carbonyl]-methanesulfonamide;
N-[3-(2-Amino-pyrimidin-4-yl)-2-(6-methyl-pyridin-2-yl)-imidazo[1,2-a]pyridine-7-carbonyl]-methanesulfonamide;
N-[3-(2-Amino-pyrimidin-4-yl)-2-(6-methyl-pyridin-2-yl)-imidazo[1,2-a]pyrimidin-7-yl]-2-(3-methoxy-phenyl)-acetamide;
N-[3-(2-Amino-pyrimidin-4-yl)-2-(6-methyl-pyridin-2-yl)-imidazo[1,2-a]pyrimidin-7-yl]-3,3-dimethyl-butyramide;
N-[3-(2-Amino-pyrimidin-4-yl)-2-(6-methyl-pyridin-2-yl)-imidazo[1,2-a]pyrimidin-7-yl]-3-pyridin-3-yl-propionamide;
N-[3-(2-Amino-pyrimidin-4-yl)-2-(6-methyl-pyridin-2-yl)-imidazo[1,2-a]pyrimidin-7-yl]-acetamide;
N-[3-(2-Amino-pyrimidin-4-yl)-2-(6-methyl-pyridin-2-yl)-imidazo[1,2-a]pyrimidin-7-yl]-nicotinamide;
N-[3-(2-Methanesulfonyl-pyrimidin-4-yl)-2-(6-methyl-pyridin-2-yl)-imidazo[1,2-a]pyrimidin-7-yl]-2-(3-methoxy-phenyl)-acetamide;
N-[3-(2-Methanesulfonyl-pyrimidin-4-yl)-2-(6-methyl-pyridin-2-yl)-imidazo[1,2-a]pyrimidin-7-yl]-3,3-dimethyl-butyramide;
N-[3-(2-Methanesulfonyl-pyrimidin-4-yl)-2-(6-methyl-pyridin-2-yl)-imidazo[1,2-a]pyrimidin-7-yl]-3-pyridin-3-yl-propionamide;
N-[3-(2-Methanesulfonyl-pyrimidin-4-yl)-2-(6-methyl-pyridin-2-yl)-imidazo[1,2-a]pyrimidin-7-yl]-nicotinamide;
N-[3-(2-Methanesulfonyl-pyrimidin-4-yl)-2-(6-methyl-pyridin-2-yl)-imidazo[1,2-a]pyrimidin-7-yl]-propionamide;
N-[3-(2-Amino-pyrimidin-4-yl)-2-(6-methyl-pyridin-2-yl)-imidazo[1,2-a]pyrimidin-7-yl]-propionamide;
N-{4-[2-(6-Methyl-pyridin-2-yl)-imidazo[1,2-a]pyridin-3-yl]-pyrimidin-2-yl}-acetamide;
N1-{4-[2-(6-Methyl-pyridin-2-yl)-imidazo[1,2-a]pyridin-3-yl]-pyrimidin-2-yl}-butane-1,4-diamine;
N1-{4-[2-(6-Methyl-pyridin-2-yl)-imidazo[1,2-a]pyridin-3-yl]-pyrimidin-2-yl}-propane-1,3-diamine;
N-(4-{4-[2-(6-Methyl-pyridin-2-yl)-imidazo[1,2-a]pyridin-3-yl]-pyrimidin-2-ylamino}-butyl)-(BODIPY FL) amide; and
N-(4-{4-[2-(6-Methyl-pyridin-2-yl)-imidazo[1,2-a]pyridin-3-yl]-pyrimidin-2-ylamino}-butyl)-(Texas Red-X) amide.

An N-oxide derivative or a pharmaceutically acceptable salt of each of the compounds of formula (I) is also within the scope of this invention. For example, a nitrogen ring atom of the imidazole core ring or a nitrogen-containing heterocyclyl substituent can form an oxide in the presence of a suitable oxidizing agent such as m-chloroperbenzoic acid or H₂O₂.

A compound of formula (I) that is acidic in nature (e.g., having a carboxyl or phenolic hydroxyl group) can form a pharmaceutically acceptable salt such as a sodium, potassium, calcium, or gold salt. Also within the scope of the invention are salts formed with pharmaceutically acceptable amines such as ammonia, alkyl amines, hydroxyalkylamines, and N-methylglycamine. A compound of formula (I) can be treated with an acid to form acid addition salts. Examples of such an acid include hydrochloric acid, hydrobromic acid, hydroiodic acid, sulfuric acid, methanesulfonic acid, phosphoric acid, p-bromophenyl-sulfonic acid, carbonic acid, succinic acid, citric acid, benzoic acid, oxalic acid, malonic acid, salicylic acid, malic acid, fumaric acid, ascorbic acid, maleic acid, acetic acid, and other mineral and organic acids well known to a skilled person in the art. The acid addition salts can be prepared by treating a compound of formula (I) in its free base form with a sufficient amount of an acid (e.g., hydrochloric acid) to produce an acid addition salt (e.g., a hydrochloride salt). The acid addition salt can be converted back to its free base form by treating the salt with a suitable dilute aqueous basic solution (e.g., sodium hydroxide, sodium bicarbonate, potassium carbonate, or ammonia). Compounds of formula (I) can also be, e.g., in a form of achiral compounds, racemic mixtures, optically active compounds, pure diastereomers, or a mixture of diastereomers.

Compounds of formula (I) exhibit surprisingly high affinity to the TGFβ family type I receptors, Alk 5 and/or Alk 4, e.g., with an IC₅₀ value of less than 10 μM under conditions as described in Examples 7 and 8 below. Some compounds of formula (I) exhibit an IC₅₀ value of below 0.1 μM.

Compounds of formula (I) can also be modified by appending appropriate functionalities to enhance selective biological properties. Such modifications are known in the art and include those that increase biological penetration into a given biological system (e.g., blood, lymphatic system, central nervous system), increase oral availability, increase solubility to allow administration by injection, alter metabolism, and/or alter rate of excretion. Examples of these modifications include, but are not limited to, esterification with polyethylene glycols, derivatization with pivolates or fatty acid substituents, conversion to carbamates, hydroxylation of aromatic rings, and heteroatom-substitution in aromatic rings.

The present invention also features a pharmaceutical composition comprising a compound of formula (I) (or a combination of two or more compounds of formula (I)) and a pharmaceutically acceptable carrier. Also included in the present invention is a medicament composition including any of the compounds of formula (I), alone or in a combination, together with a suitable excipient.

The invention also features a method of inhibiting the TGFβ family type I receptors, Alk 5 and/or Alk 4 (e.g., with an IC₅₀ value of less than 10 μM; preferably, less than 1 μM; more preferably, less than 0.1 μM in a cell, including the step of contacting the cell with an effective amount of one or more compounds of formula (I). Also with the scope of the invention is a method of inhibiting the TGFβ and/or activin signaling pathway in a cell or in a subject (e.g., a mammal such as human), including the step of contacting the cell with or administering to the subject an effective amount of one or more of a compound of formula (I).

Also within the scope of the present invention is a method of treating a subject or preventing a subject from suffering a condition characterized by or resulted from an elevated level of TGFβ and/or activin activity (e.g., from an overexpression of TGFβ). The method includes the step of administering to the subject an effective amount of one or more of a compound of formula (I). The conditions include an accumulation of excess extracellular matrix; a fibrotic condition (e.g., scleroderma, lupus nephritis, connective tissue disease, wound healing, surgical scarring, spinal cord injury, CNS scarring, acute lung injury, idiopathic pulmonary fibrosis, chronic obstructive pulmonary disease, adult respiratory distress syndrome, acute lung injury, drug-induced lung injury, glomerulonephritis, diabetic nephropathy, hypertension-induced nephropathy, hepatic or biliary fibrosis, liver cirrhosis, primary biliary cirrhosis, fatty liver disease, primary sclerosing cholangitis, restenosis, cardiac fibrosis, opthalmic scarring, fibrosclerosis, fibrotic cancers, fibroids, fibroma, fibroadenomas, fibrosarcomas, transplant arteriopathy, and keloid); demyelination of neurons multiple sclerosis; Alzheimer's disease; cerebral angiopathy; and TGFβ-induced metastasis of tumor cells and carcinomas (e.g, squamous cell carcinomas, multiple myeloma, melanoma, glioma, glioblastomas, leukemia, and carcinomas of the lung, breast, ovary, cervix, liver, biliary tract, gastrointestinal tract, pancreas, prostate, and head and neck).

As used herein, an “alkyl” group refers to a saturated aliphatic hydrocarbon group containing 1-8 (e.g., 1-6 or 1-4) carbon atoms. An alkyl group can be straight or branched. Examples of an alkyl group include, but are not limited to, methyl, ethyl, propyl, isopropyl, butyl, isobutyl, sec-butyl, tert-butyl, n-pentyl, n-heptyl, and 2-ethylhexyl. An alkyl group can be optionally substituted with one or more substituents such as alkoxy, cycloalkyloxy, heterocycloalkyloxy, aryloxy, heteroaryloxy, aralkyloxy, heteroarylalkoxy, amino, nitro, carboxy, cyano, halo, hydroxy, sulfo, mercapto, alkylsulfanyl, alkylsulfinyl, alkylsulfonyl, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, urea, thiourea, sulfamoyl, sulfamide, alkoxycarbonyl, or alkylcarbonyloxy.

As used herein, an “alkenyl” group refers to an aliphatic carbon group that contains 2-8 (e.g., 2-6 or 2-4) carbon atoms and at least one double bond. Like an alkyl group, an alkenyl group can be straight or branched. Examples of an alkenyl group include, but are not limited to, allyl, isoprenyl, 2-butenyl, and 2-hexenyl. An alkenyl group can be optionally substituted with one or more substituents such as alkoxy, cycloalkyloxy, heterocycloalkyloxy, aryloxy, heteroaryloxy, aralkyloxy, heteroarylalkoxy, amino, nitro, carboxy, cyano, halo, hydroxy, sulfo, mercapto, alkylsulfanyl, alkylsulfinyl, alkylsulfonyl, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, urea, thiourea, sulfamoyl, sulfamide, alkoxycarbonyl, or alkylcarbonyloxy.

As used herein, an “alkynyl” group refers to an aliphatic carbon group that contains 2-8 (e.g., 2-6 or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as alkoxy, cycloalkyloxy, heterocycloalkyloxy, aryloxy, heteroaryloxy, aralkyloxy, heteroarylalkoxy, amino, nitro, carboxy, cyano, halo, hydroxy, sulfo, mercapto, alkylsulfanyl, alkylsulfinyl, alkylsulfonyl, aminocarbonyl, alkylcarbonylamino, cycloalkylcarbonylamino, cycloalkyl-alkylcarbonylamino, arylcarbonylamino, aralkylcarbonylamino, heterocycloalkyl-carbonylamino, heterocycloalkyl-alkylcarbonylamino, heteroarylcarbonylamino, heteroaralkylcarbonylamino, urea, thiourea, sulfamoyl, sulfamide, alkoxycarbonyl, or alkylcarbonyloxy.

As used herein, an “amino” group refers to —NR^{X}R^{Y} wherein each of R^{X} and R^{Y} is independently hydrogen, hydroxyl, alkyl, alkoxy, cycloalkyl, (cycloalkyl)alkyl, aryl, aralkyl, heterocycloalkyl, (heterocycloalkyl)alkyl, heteroaryl, or heteroaralkyl. When the term “amino” is not the terminal group (e.g., alkylcarbonylamino), it is represented by —NR^{X}—. R^{X} has the same meaning as defined above.

As used herein, an “aryl” group refers to phenyl, naphthyl, or a benzofused group having 2 to 3 rings. For example, a benzofused group includes phenyl fused with one or two C₄₋₈ carbocyclic moieties, e.g., 1, 2, 3, 4-tetrahydronaphthyl, indanyl, or fluorenyl. An aryl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, (heterocycloalkyl)carbonylamino, (heterocycloalkyl)alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl.

As used herein, a “cycloalkyl” group refers to an aliphatic carbocyclic ring of 3-10 (e.g., 4-8) carbon atoms. Examples of cycloalkyl groups include cyclopropyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, and bicyclo[3.2.3]nonyl. A “cycloalkenyl” group, as used herein, refers to a non-aromatic carbocyclic ring of 3-10 (e.g., 4-8) carbon atoms having one or more double bond. Examples of cycloalkenyl groups include cyclopentenyl, 1,4-cyclohexa-di-enyl, cycloheptenyl, cyclooctenyl, hexahydro-indenyl, octahydro-naphthyl, bicyclo[2.2.2]octenyl, and bicyclo[3.3.1]nonenyl. A cycloalkyl or cycloalkenyl group can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, heterocycloalkyl-carbonylamino, (heterocycloalkyl)alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, a “heterocycloalkyl” group refers to a 3- to 10-membered (e.g., 4- to 8-membered) saturated ring structure, in which one or more of the ring atoms is a heteroatom, e.g., N, O, or S. Examples of a heterocycloalkyl group include piperidinyl, piperazinyl, tetrahydropyranyl, tetrahydrofuryl, dioxolanyl, oxazolidinyl, isooxazolidinyl, morpholinyl, octahydro-benzofuryl, octahydro-chromenyl, octahydro-thiochromenyl, octahydro-indolyl, octahydro-pyrindinyl, decahydro-quinolinyl, octahydro-benzo[b]thiophenyl, 2-oxa-bicyclo[2.2.2]octyl, 1-aza-bicyclo[2.2.2]octyl, 3-aza-bicyclo[3.2.1]octyl, anad 2,6-dioxa-tricyclo[3.3.1.0^{3,7}]nonyl. A “heterocycloalkenyl” group, as used herein, refers to a 3- to 10-membered (e.g., 4- to 8-membered) non-aromatic ring structure having one or more double bonds, and wherein one or more of the ring atoms is a heteroatom, e.g., N, O, or S. A heterocycloalkyl or heterocycloalkenyl group can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, heterocycloalkyl-carbonylamino, (heterocycloalkyl)alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

A “heteroaryl” group, as used herein, refers to a monocyclic, bicyclic, or tricyclic ring structure having 5 to 15 ring atoms wherein one or more of the ring atoms is a heteroatom, e.g., N, O, S, or B and wherein one ore more rings of the bicyclic or tricyclic ring structure is aromatic. Some examples of heteroaryl are pyridyl, furyl, pyrrolyl, thienyl, thiazolyl, oxazolyl, imidazolyl, indolyl, tetrazolyl, benzofuryl, benzthiazolyl, xanthene, thioxanthene, phenothiazine, dihydroindole, and benzo[1,3]dioxole. A heteroaryl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, amino, nitro, carboxy, alkoxycarbonyl, alkylcarbonyloxy, aminocarbonyl, alkylcarbonylamino, cycloalkylcarbonylamino, (cycloalkyl)alkylcarbonylamino, arylcarbonylamino, aralkylcarbonylamino, heterocycloalkyl-carbonylamino, (heterocycloalkyl)alkylcarbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl. A “heteroaralkyl” group, as used herein, refers to an alkyl group (e.g., a C₁₋₄ alkyl group) that is substituted with a heteroaryl group. Both “alkyl” and “heteroaryl” have been defined above.

As used herein, “cyclic moiety” includes cycloalkyl, heterocycloalkyl, cycloalkenyl, heterocycloalkenyl, aryl, or heteroaryl, each of which has been defined previously.

As used herein, an “acyl” group refers to a formyl group or alkyl-C(═O)— where “alkyl” has been defined previously. Acetyl and pivaloyl are examples of acyl groups.

As used herein, a “carbamoyl” group refers to a group having the structure —O—CO—NR^{X}R^{Y} or —NR^{X}—CO—R^{Z} wherein R^{X} and R^{Y} have been defined above and R^{Z} is alkyl, cycloalkyl, (cycloalkyl)alkyl, aryl, aralkyl, heterocycloalkyl, (heterocycloalkyl)alkyl, heteroaryl, or heteroaralkyl.

As used herein, a “carboxy” and a “sulfo” group refer to —COOH and —SO₃H, respectively.

As used herein, an “alkoxy” group refers to an alkyl-O— group where “alkyl” has been defined previously.

As used herein, a “sulfoxy” group refers to —O—SO—R^{X} or —SO—R^{X}, where R^{X} has been defined above.

As used herein, a “halogen” or “halo” group refers to fluorine, chlorine, bromine or iodine.

As used herein, a “sulfamoyl” group refers to the structure —S(O)₂—NRxRY or —NR^{X}—S(O)₂—R^{Z} wherein R^{X}, R^{Y}, and R^{Z} have been defined above.

As used herein, a “sulfamide” group refers to the structure —NR^{X}—S(O)₂—NR^{Y}R^{Z} wherein R^{X}, R^{Y}, and R^{Z} have been defined above.

As used herein, a “urea” group refers to the structure —NR^{X}—CO—NR^{Y}R^{Z} and a “thiourea” group refers to the structure —NR^{X}—CS—NR^{Y}R^{Z}. R^{X}, R^{Y}, and R^{Z} have been defined above.

As used herein, an effective amount is defined as the amount which is required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, N.Y., 537 (1970). As used herein, “patient” refers to a mammal, including a human.

An antagonist is a molecule that binds to the receptor without activating the receptor. It competes with the endogenous ligand(s) or substrate(s) for binding site(s) on the receptor and, thus inhibits the ability of the receptor to transduce an intracellular signal in response to endogenous ligand binding.

As compounds of formula (I) are antagonists of TGFβ receptor type I (Alk5) and/or activin receptor type I (Alk4), these compounds are useful in inhibiting the consequences of TGFβ and/or activin signal transduction such as the production of extracellular matrix (e.g., collagen and fibronectin), the differentiation of stromal cells to myofibroblasts, and the stimulation of and migration of inflammatory cells. Thus, compounds of formula (I) inhibit pathological inflammatory and fibrotic responses and possess the therapuetical utility of treating and/or preventing disorders or diseases for which reduction of TGFβ and/or activin activity is desirable (e.g., various types of fibrosis or progressive cancers).

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

Other features and advantages of the invention will be apparent from the following detailed description, and from the claims.

### DETAILED DESCRIPTION OF THE INVENTION

In general, the invention features compounds of formula (I), which exhibit surprisingly high affinity for the TGFβ family type I receptors, Alk 5 and/or Alk 4.

Synthesis of Compounds of Formula (I)

Compounds of formula (I) may be prepared by a number of known methods from commercially available or known starting materials. In one method, compounds of formula (I) are prepared according to Scheme 1 below. Specifically, a starting compound of formula (II) (where R² has been selected beforehand) can be methylated in the presence of methyl iodide under a basic condition (e.g., aq. NaOH) to yield form a compound of formula (III), which can be deprotonated under appropriate conditions (e.g., using sodium hexamethyldisilazane (NaHMS) in THF). Reaction of the deprotonated compound of formula (III) with a compound of formula (IV) (where R¹ has been selected beforehand) leads to an adduct compound of formula (V). The adduct is then brominated and then cyclize with an amino-subsituted heterocycle of formula (VI) to yield a compound of formula (I).

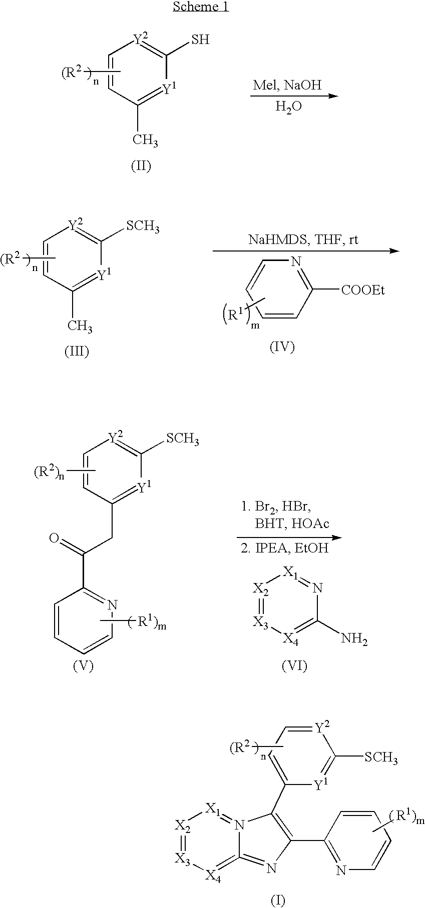

Referring to Scheme 2 below, the thioether-substituted compound of formula (I) (see end product in Scheme 1) can be further modified to form other compounds of formula (I). Note that each of R^{A} and R^{B} represents hydrogen, alkyl, cycloalkyl, (cycloalkyl)alkyl, aryl, aralkyl, heterocycloalkyl, (heterocycloalkyl)alkyl, heteroaryl, or heteroaralkyl.

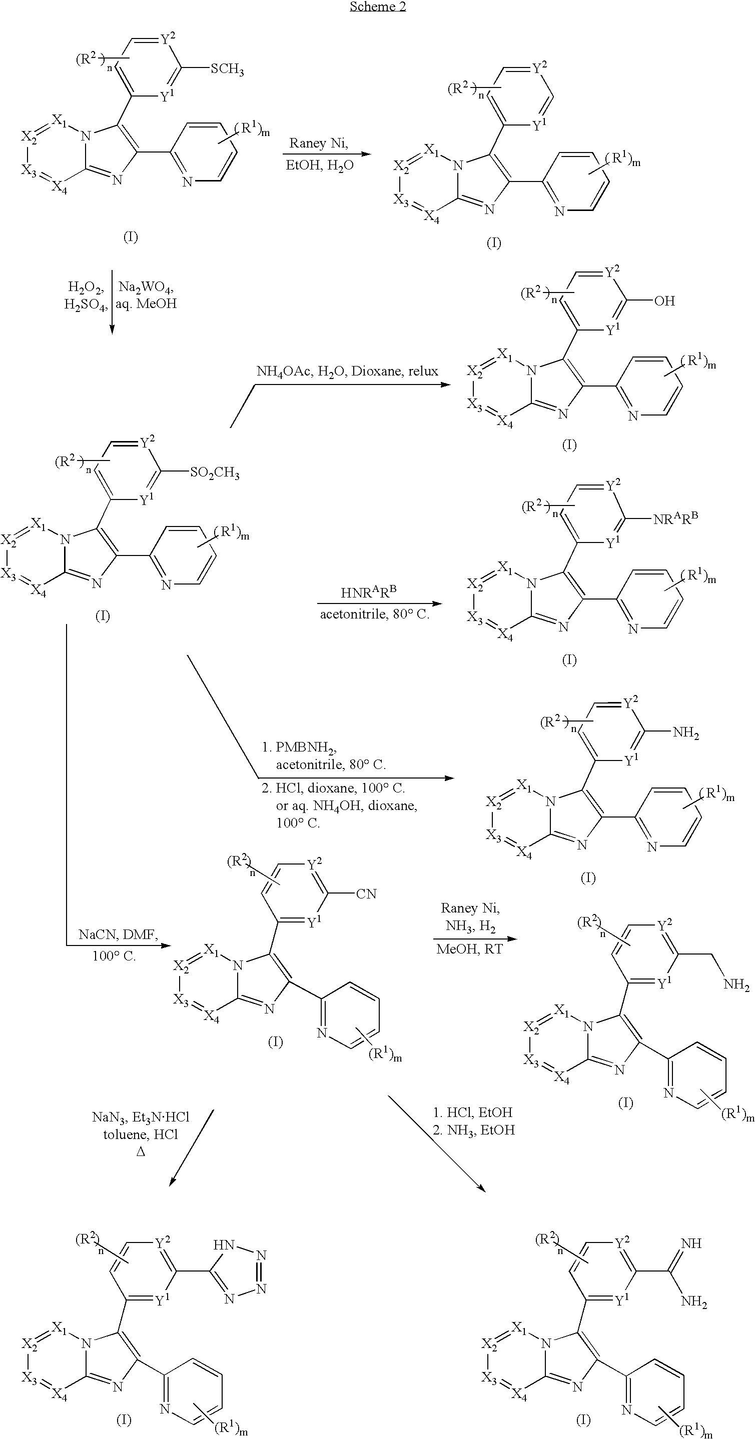

Alternatively, compounds of formula (I) can be prepared according to Scheme 3 below. Specifically, a compound of formula (VII) can cyclize with an amino-substituted heterocycle of formula (VI) to yield a compound of formula (VII), which can be brominated to form a compound of formula (IX). Compounds of formula (IX) and formula (X) can undergo a Suzuki coupling reaction to yield a compound of formula (I), which can be further modified to form other compounds of formula (I). See the amination reaction as illustrated in the last step of Scheme 3 above (each of R^{A} and R^{B} having the same meaning as provided above). For preparation of a compound of formula (X), see WO 02/16359.

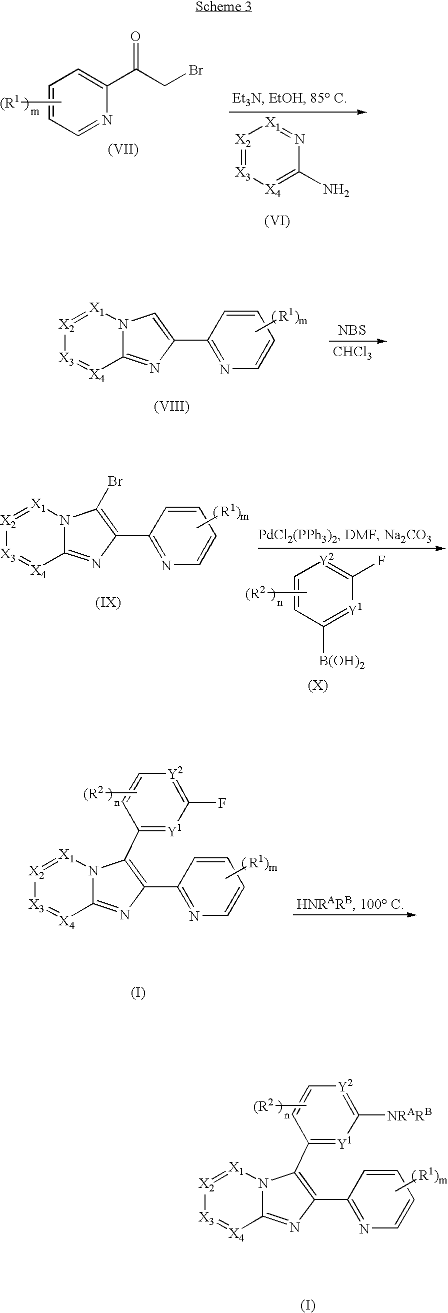

Alternatively, a bromo-substituted compound of formula (I) can be modified to other compounds of formula (I) according to Scheme 4 below.

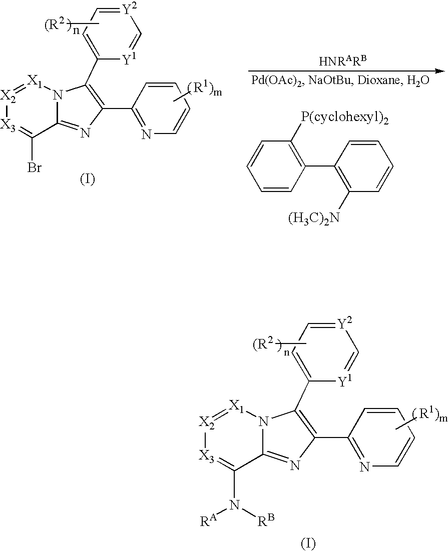

Alternatively, an amino-substituted compound of formula (I) can be modified to other compounds of formula (I) according to Scheme 5 below, where R^{C} represents alkyl, cycloalkyl, (cycloalkyl)alkyl, aryl, aralkyl, heterocycloalkyl, (heterocycloalkyl)alkyl, heteroaryl, or heteroaralkyl.

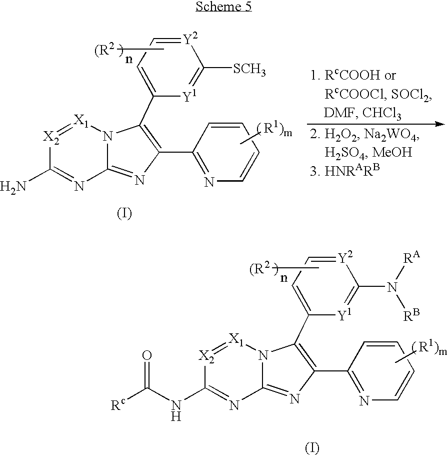

It should be noted that the methods illustrated in Schemes above are merely examples. One skilled in the art can easily modify these methods to produce compounds of formula (I) other than those shown in the Schemes. For example, one can replace the mono(methylsulfanyl)-substituted compound of formula (III) in Scheme 1 with a di(methylsulfanyl)-substituted derivative (e.g., 4-methyl-2,6-bis-methylsulfanyl-pyrimidine, which can be prepared according to, e.g., Aust. J. Chem. 34:1729 (1981), Syn. Commun. 10:791 (1980), or Synthesis 70-72 (1988)), thus resulting in various di-substituted compounds of formula (I) (i.e., disubstituted derivatives of compounds of formula (I) as shown in Schemes 1 and 2).

In one embodiment of the invention the molecular weight of the inventive compound is no more than 1200. In another embodiment of the invention the molecular weight is no more than 1000.

As will be obvious to a skilled person in the art, some intermediates may need to be protected before undergoing synthetic steps as described above. For suitable protecting groups, see, e.g., T. W. Greene, Protective Groups in Organic Synthesis, John Wiley & Sons, Inc., New York (1981).

Uses of Compounds of Formula (I)

As discussed above, hyperactivity of the TGFβ family signaling pathways can result in excess deposition of extracellular matrix and increased inflammatory responses, which can then lead to fibrosis in tissues and organs (e.g., lung, kidney, and liver) and ultimately result in organ failure. See, e.g., Border, W. A. and Ruoslahti E. J. Clin. Invest. 90:1-7 (1992) and Border, W. A. and Noble, N. A. N. Engl. J. Med. 331: 1286-1292 (1994). Studies have been shown that the expression of TGFβ and/or activin mRNA and the level of TGFβ and/or activin are increased in patients suffering from various fibrotic disorders, e.g., fibrotic kidney diseases, alcohol-induced and autoimmune hepatic fibrosis, myelofibrosis, bleomycin-induced pulmonary fibrosis, and idiopathic pulmonary fibrosis.

Compounds of formula (I), which are antagonists of the TGFβ family type I receptors, Alk 5 and/or Alk 4, and inhibit TGFβ and/or activin signaling pathway, are therefore useful for treating and/or preventing fibrotic disorders or diseases mediated by an increased level of TGFβ and/or activin activity. As used herein, a compound inhibits the TGFβ family signaling pathway when it binds (e.g., with an IC₅₀ value of less than 10 μM; preferably, less than 1 μM; more preferably, less than 0.1× to a receptor of the pathway (e.g., Alk 5 and/or Alk 4), thereby competing with the endogenous ligand(s) or substrate(s) for binding site(s) on the receptor and reducing the ability of the receptor to transduce an intracellular signal in response to the endogenous ligand or substrate binding. The aforementioned disorders or diseases include any conditions (a) marked by the presence of an abnormally high level of TGFβ and/or activin; and/or (b) an excess accumulation of extracellular matrix; and/or (c) an increased number and synthetic activity of myofibroblasts. These disorders or diseases include, but are not limited to, fibrotic conditions such as scleroderma, idiopathic pulmonary fibrosis, glomerulonephritis, diabetic nephropathy, lupus nephritis, hypertension-induced nephropathy, ocular or corneal scarring, hepatic or biliary fibrosis, acute lung injury, pulmonary fibrosis, post-infarction cardiac fibrosis, fibrosclerosis, fibrotic cancers, fibroids, fibroma, fibroadenomas, and fibrosarcomas. Other fibrotic conditions for which preventive treatment with compounds of formula (I) can have therapeutic utility include radiation therapy-induced fibrosis, chemotherapy-induced fibrosis, surgically induced scarring including surgical adhesions, laminectomy, and coronary restenosis.

Increased TGFβ activity is also found to manifest in patients with progressive cancers. Studies have shown that in late stages of various cancers, both the tumor cells and the stromal cells within the tumors generally overexpress TGFβ. This leads to stimulation of angiogenesis and cell motility, suppression of the immune system, and increased interaction of tumor cells with the extracellular matrix. See, e.g., Hojo, M. et al., Nature 397: 530-534 (1999). As a result, the tumors cells become more invasive and metastasize to distant organs. See, e.g., Maehara, Y. et al., J. Clin. Oncol. 17: 607-614 (1999) and Picon, A. et al., Cancer Epidemiol. Biomarkers Prev. 7: 497-504 (1998). Thus, compounds of formula (I), which are antagonists of the TGFβ type I receptor and inhibit TGFβ signaling pathway, are also useful for treating and/or preventing various late stage cancers which overexpress TGFβ. Such late stage cancers include carcinomas of the lung, breast, liver, biliary tract, gastrointestinal tract, head and neck, pancreas, prostate, cervix as well as multiple myeloma, melanoma, glioma, and glioblastomas.

Importantly, it should be pointed out that because of the chronic and in some cases localized nature of disorders or diseases mediated by overexpression of TGFβ and/or activin (e.g., fibrosis or cancers), small molecule treatments (such as treatment disclosed in the present invention) are favored for long-term treatment.

Not only are compounds of formula (I) useful in treating disorders or diseases mediated by high levels of TGFβ and/or activin activity, these compounds can also be used to prevent the same disorders or diseases. It is known that polymorphisms leading to increased TGFβ and/or activin production have been associated with fibrosis and hypertension. Indeed, high serum TGFβ levels are correlated with the development of fibrosis in patients with breast cancer who have received radiation therapy, chronic graft-versus-host-disease, idiopathic interstitial pneumonitis, veno-occlusive disease in transplant recipients, and peritoneal fibrosis in patients undergoing continuous ambulatory peritoneal dialysis. Thus, the levels of TGFβ and/or activin in serum and of TGFβ and/or activin mRNA in tissue can be measured and used as diagnostic or prognostic markers for disorders or diseases mediated by overexpression of TGFβ and/or activin, and polymorphisms in the gene for TGFβ that determine the production of TGFβ and/or activin can also be used in predicting susceptibility to disorders or diseases. See, e.g., Blobe, G. C. et al., N. Engl. J. Med. 342(18): 1350-1358 (2000); Matsuse, T. et al., Am. J. Respir. Cell Mol. Biol. 13: 17-24 (1995); Inoue, S. et al., Biochem. Biophys. Res. Comm. 205: 441-448 (1994); Matsuse, T. et al, Am. J. Pathol. 148: 707-713 (1996); De Bleser et al., Hepatology 26: 905-912 (1997); Pawlowski, J. E., et al., J. Clin. Invest. 100: 639-648 (1997); and Sugiyama, M. et al., Gastroenterology 114: 550-558 (1998).

Administration of Compounds of Formula (I)

As defined above, an effective amount is the amount which is required to confer a therapeutic effect on the treated patient. For a compound of formula (I), an effective amount can range from about 1 mg/kg to about 150 mg/kg (e.g., from about 1 mg/kg to about 100 mg/kg). Effective doses will also vary, as recognized by those skilled in the art, dependant on route of administration, excipient usage, and the possibility of co-usage with other therapeutic treatments including use of other therapeutic agents and/or radiation therapy.

Compounds of formula (I) can be administered in any manner suitable for the administration of pharmaceutical compounds, including, but not limited to, pills, tablets, capsules, aerosols, suppositories, liquid formulations for ingestion or injection or for use as eye or ear drops, dietary supplements, and topical preparations. The pharmaceutically acceptable compositions include aqueous solutions of the active agent, in a isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the therapeutic compounds. As to route of administration, the compositions can be administered orally, intranasally, transdermally, intradermally, vaginally, intraaurally, intraocularly, buccally, rectally, transmucosally, or via inhalation, implantation (e.g., surgically), or intravenous administration. The compositions can be administered to an animal (e.g., a mammal such as a human, non-human primate, horse, dog, cow, pig, sheep, goat, cat, mouse, rat, guinea pig, rabbit, hamster, gerbil, ferret, lizard, reptile, or bird).

Optionally, compounds of formula (I) can be administered in conjunction with one or more other agents that inhibit the TGFβ signaling pathway or treat the corresponding pathological disorders (e.g., fibrosis or progressive cancers) by way of a different mechanism of action. Examples of these agents include angiotensin converting enzyme inhibitors, nonsteroid, steroid anti-inflammatory agents, and chemotherapeutics or radiation, as well as agents that antagonize ligand binding or activation of the TGFβ receptors, e.g., anti-TGFβ, anti-TGFβ receptor antibodies, or antagonists of the TGFβ type II receptors.

The invention will be further described in the following examples, which do not limit the scope of the invention described in the claims.

### EXAMPLE 1

### 2-(6-Methyl-pyridin-2-yl)-3-(2-methylsulfanyl-pyrimidin-4-yl)-imidazo[1,2-a]pyridine

Synthesis of the title compound is described in parts (a)-(c) below.

### (a) 4-Methyl-2-methylsulfanyl-pyrimidine

Crushed sodium hydroxide (272.7 mmol) was added to a slurry of 2-mercapto-4-methylpyrimidine HCl (122.8 mmol) in water (175 mL) at room temperature. After 15 minutes, iodomethane (134.9 mmol) was added dropwise to the dark brown solution with formation of an orange precipitate. The slurry was stirred at room temperature for 3.5 hours and then extracted with methylene chloride (3×60 mL). The combined organic phases were dried (Na₂SO₄) and concentrated in vacuo to give 17.141 g of a dark brown oil identified as 4-methyl-2-methylsulfanyl-pyrimidine. ¹H NMR (CDCl₃, 300 MHz): 2.41 (s, 3H), 2.52 (s, 3H), 6.77 (d, J=5.06 Hz, 1H), 8.32 (d, J=5.12 Hz, 1H); MS (ESP+) 141.14 (M+1).

### (b) 1-(6-Methyl-pyridin-2-yl)-2-(2-methylsulfanyl-pyrimidin-4-yl)-ethanone

Sodium bis(trimethylsilyl)amide/THF (1.0 M, 74 mmol) was added dropwise to a solution of 4-methyl-2-methylsulfanyl-pyrimidine (36.89 mmol) and 6-methyl-pyridine-2-carboxylic acid ethyl ester (36.80 mmol) in anhydrous THF (74 mL) under a nitrogen atmosphere in a room temperature water bath. After stirring for 3 hours, the reaction was quenched with saturated NH₄Cl (110 mL) and the organic and aqueous phases were separated. The aqueous phase was extracted with ethyl acetate (2×100 mL). The combined organic phases were washed with water (100 mL) and brine (100 mL), dried (MgSO₄), and concentrated in vacuo to give a dark orange solid, which was then slurried in ether at room temperature for 1 hour, cooled at 0° C. overnight, filtered, and air dried to give 4.85 g of a dark brown solid identified as the enol of 1-(6-methyl-pyridin-2-yl)-2-(2-methylsulfanyl-pyrimidin-4-yl)-ethanone. ¹H NMR (CDCl₃, 400 MHz): 2.60 (s, 6H), 6.73 (s, 1H), 6.76 (d, J=5.35 Hz, 1H), 7.17 (d, J=7.54 Hz, 1H), 7.67 (dd, J=7.72, 7.72 Hz, 1H), 7.75 (d, J=7.72 Hz, 1H), 8.33 (d, J=5.37 Hz, 1H), 14.30 (s, 1H); MS (ESP+) 260.16 (M+1). The ether solution was concentrated it vacuo, dissolved in ethyl acetate, treated with decolorizing carbon, filtered and concentrated in vacuo to give a solid, which was slurried in ether at room temperature for 1 hour, cooled at 0° C. overnight, filtered, and air dried to give 0.63 g of a light brown solid identified as a 4:1 mixture of the ketone/enol form of 1-(6-methyl-pyridin-2-yl)-2-(2-methylsulfanyl-pyrimidin-4-yl)-ethanone.

### (c) 2-(6-Methyl-pyridin-2-yl)-3-(2-methylsulfanyl-pyrimidin-4-yl)-imidazo[1,2-a]pyridine

Hydrogen bromide/acetic acid (30 wt %, 2.07 mmol) and 0.99 M bromine/acetic acid (1.21 mmol) was added to a solution of 1-(6-methyl-pyridin-2-yl)-2-(2-methylsulfanyl-pyrimidin-4-yl)-ethanone (0.89 mmol) and catalytic BHT in glacial acetic acid (4 mL) at room temperature to form a precipitate. After 1 hour of stirring, the reaction was diluted to 50 mL with ether, filtered, washed with ether, air dried briefly and then dissolved in ethanol (6 mL). The ethanolic solution was added dropwise to a solution of 2-aminopyridine (0.965 mmol) and diisopropylethylamine (2.67 mmol) in ethanol (1 mL) at 67° C. After stirring 3.5 hours, the reaction was concentrated in vacuo and partioned between ether (20 mL) and 1 M HCl (10 mL). The aqueous phase was washed with ether (2×10 mL), cooled in an ice bath and solid sodium bicarbonate was added until the solution was neutral. The slurry was cooled at 0° C., filtered and air dried to give 0.18 g of brown solid, which was identified as the title compound, 2-(6-methyl-pyridin-2-yl)-3-(2-methylsulfanyl-pyrimidin-4-yl)-imidazo[1,2-a]pyridine. ¹H NMR (CDCl₃, 300 MHz): 2.53 (s, 3H), 2.62 (s, 3H), 6.94 (dd, J=7.18, 6.73 Hz, 1H), 7.14 (d, J=5.42 Hz, 1H), 7.18 (d, J=4.49 Hz, 1H), 7.36 (dd, J=7.44, 8.35 Hz, 1H), 7.67 (d, J=4.43 Hz, 1H), 7.71 (dd, J=8.96 Hz, 2H), 8.33 (d, J=5.38 Hz, 1H), 9.49 (d, J=7.09 Hz, 1H); MS (ESP+) 334.15 (M+1).

### EXAMPLE 2

### 3-(2-Methanesulfonyl-pyrimidin-4-yl)-2-(6-methyl-pyridin-2-yl)-imidazo[1,2-a]pyridine

Sulfuric acid (4.0 N, 0.04 mmol), catalytic sodium tungstate dihydrate, and 30 wt % hydrogen peroxide (1.01 mmol) was added to a slurry of 2-(6-methyl-pyridin-2-yl)-3-(2-methylsulfanyl-pyrimidin-4-yl)-imidazo[1,2-a]pyridine (0.31 mmol; see Example 1 for its preparation) in methanol (2 mL) at 55° C. All solids dissolved at the beginning of the reaction, then a precipitate formed. After 3 hours of stirring, the reaction was diluted with water (2 mL) and warmed to 55° C. for a further 0.5 hour. The reaction was then cooled to room temperature, quenched with saturated sodium thiosulfate and concentrated in vacuo. The solid was partioned between ethyl acetate (20 mL) and water (10 mL). The organic phase was washed with 2M sodium carbonate (7 mL) and brine (7 mL), dried (Na₂SO₄) and concentrated in vacuo to give 0.10 g of a yellow solid identified as the title compound, 3-(2-methanesulfonyl-pyrimidin-4-yl)-2-(6-methyl-pyridin-2-yl)-imidazo[1,2-a]pyridine. ¹H NMR (CDCl₃, 400 MHz): 2.54 (s, 3H), 3.39 (s, 3H), 7.12 (dd, J=6.93, 7.76 Hz, 1H), 7.25 (d, J=7.61 Hz, 1H), 7.50 (dd, J=7.32, 7.67 Hz, 1H), 7.77 (dd, J=7.73, 7.73 Hz, 1H), 7.83 (d, J=8.91 Hz, 1H), 7.89 (d, J=9.02 Hz, 1H), 7.90 (d, J=5.58 Hz, 1H), 8.66 (d, J=5.55 Hz, 1H), 9.74 (d, J=7.08 Hz, 1H); MS (ESP+) 366.09 (M+1).

### EXAMPLE 3

### (4-Methoxy-benzyl)-{4-[2-(6-methyl-pyridin-2-yl)-imidazo[1,2-a]pyridin-3-yl]-pyrimidin-2-yl}-amine

A slurry of 4-methoxybenzylamine (0.54 mmol) and 3-(2-methanesulfonyl-pyrimidin-4-yl)-2-(6-methyl-pyridin-2-yl)-imidazo[1,2-a]pyridine (0.27 mmol; see Example 2 for its preparation) in acetonitrile (1 mL) was warmed to reflux. After refluxing for 20 hours, 4-methoxybenzylamine (0.27 mmol) was added. After refluxing for an additional 4.5 hours, the reaction was allowed to cool to room temperature and concentrated in vacuo to give a yellow solid. This solid was dissolved in chloroform (20 mL), washed with 5% citric acid (6 mL), 10% sodium bicarbonate (6 mL) and brine (7 mL), dried (Na₂SO₄) and concentrated in vacuo to give 0.11 g of a yellow wax, which was identified as the title compound, (4-methoxy-benzyl)-{4-[2-(6-methyl-pyridin-2-yl)-imidazo[1,2-a]pyridin-3-yl]-pyrimidin-2-yl}-amine. ¹H NMR (CDCl₃, 300 MHz): 2.57 (s, 3H), 3.82 (s, 3H), 4.65 (d, J=5.9 Hz, 2H), 6.65 (d, J=5.3 Hz, 1H), 6.6-6.71 (br m, 1H), 6.89-6.94 (m, 2H), 7.18 (br d, J=5.3 Hz, 1H), 7.25-7.28 (m, 2H), 7.31-7.36 (m, 2), 7.89 (br d, J=5.6 Hz, 1H), 7.93 (d, J=8.1 Hz, 1H), 7.97 (br d, J=9.6 Hz, 1H), 8.13 (d, J=5.3 Hz, 1H), 9.16 (br s, 1H); MS (ESP+) 423.20 (M+1); MS (ESP−) 421.21 (M−1).

### EXAMPLE 4

### 4-[2-(6-Methyl-pyridin-2-yl)-imidazo[1,2-a]pyridin-3-yl]-pyrimidin-2-ylamine

A solution of (4-methoxy-benzyl)-{4-[2-(6-methyl-pyridin-2-yl)-imidazo[1,2-a]pyridin-3-yl]-pyrimidin-2-yl}-amine (0.2627 mmol; see Example 3 for its preparation) in 1:1 dioxane/5N HCl (4 mL) was warmed to 100° C. for 2 days. The resultant solution was then concentrated in vacuo and purified by reverse phase HPLC (acetonitrile/water gradient with 0.1% TFA) to give 0.02 g of a yellow solid, which was identified as the TFA salt of the title compound, 4-[2-(6-methyl-pyridin-2-yl)-imidazo[1,2-a]pyridin-3-yl]-pyrimidin-2-ylamine. ¹H (d6-DMSO, 400 MHz): 2.48 (s, 3H), 6.75 (d, J=5.9 Hz, 1H), 7.20 (ddd, J=1.1, 6.9, 6.9 Hz, 1), 7.39 (d, J=7.7 Hz, 1H), 7.62 (ddd, J=1.1, 6.9, 6.9 Hz, 1H), 7.77 (d, J=7.7 Hz, 1H), 7.83 (d, J=8.9 Hz, 1H), 7.89 (dd, J=7.7, 7.7 Hz, 1H), 8.18 (d, J=5.9 Hz, 1H), 9.59 (d, J=7 Hz, 1H); MS (ESP+) 303.11 (M+1).

### EXAMPLE 5

### 3-(2-Amino-pyrimidin-4-yl)-2-(6-methyl-pyridin-2-yl)-imidazo[1,2-a]pyrimidin-7-ylamine

A solution of 3-(2-methanesulfonyl-pyrimidin-4-yl)-2-(6-methyl-pyridin-2-yl)-imidazo[1,2-a]pyrimidin-7-ylamine (0.0718 mmol; prepared in accordance with Example 2) and 28 wt % ammonium hydroxide (4.34 mmol) in dioxane (2 mL) was warmed to 100° C. in a sealed tube. After 19.5 hours, the reaction was concentrated in vacuo and purified via reverse phase HPLC (acetonitrile/water gradient with 0.1% TFA) to give 0.0200 g of a yellow solid identified as the TFA salt of the title compound, 3-(2-amino-pyrimidin-4-yl)-2-(6-methyl-pyridin-2-yl)-imidazo[1,2-a]pyrimidin-7-ylamine. ¹H NMR (DMSO-d6, 400 MHz): 2.49 (s, 1.5H), 2.50 (s, 1.5H), 6.54 (d, J=5.37 Hz, 1H), 6.64 (d, J=7.71 Hz, 1H), 7.15 (br s, 2H), 7.40 (d, J=7.75 Hz, 1H), 7.55 (d, J=7.74 Hz, 1H), 7.84 (dd, J=7.79, 7.17 Hz, 1H), 8.17 (d, J=5.37 Hz, 1H), 8.18 (br s, 2H), 9.32 (d, J=7.53 Hz, 1H); MS (ESP+) 319.18 (M+1).

### EXAMPLE 6

### 2-(6-Methyl-pyridin-2-yl)-3-(2-morpholin-4-yl-pyrimidin-4-yl)-imidazo[1,2-a]pyridine

A solution of 3-(2-methanesulfonyl-pyrimidin-4-yl)-2-(6-methyl-pyridin-2-yl)-imidazo[1,2-a]pyridine (0.121 mmol; see Example 2 for its preparation) and 4-aminomorpholine (0.38 mmol) in anhydrous acetonitrile (1 mL) was warmed to 100° C. in a sealed tube. After 3 days, 4-aminomorpholine (0.76 mmol) was added and the reaction warmed at 100° C. for an additional day. The reaction was then concentrated in vacuo and purified via reverse phase HPLC (water/acetonirile gradient with 0.1% TFA) to give 0.0258 g of an orange solid identified as the TFA salt of the title compound, 2-(6-methyl-pyridin-2-yl)-3-(2-morpholin-4-yl-pyrimidin-4-yl)-imidazo[1,2-a]pyridine. ¹H NMR (DMSO-d6, 400 MHz): 2.43 (s, 3H), 3.60-3.71 (m, 4), 6.75 (d, J=6.75 Hz, 1H), 7.16 (dd, J=6.79, 7.11 Hz, 1H), 7.37 (d, J=7.70 Hz, 1), 7.57 (dd, J=7.57, 8.26 Hz, 1H), 7.70 (d, J=7.75 Hz, 1H), 7.77 (d, J=9.01 Hz, 1H), 7.86 (dd, J=7.74, 7.78 Hz, 1H), 8.35 (d, J=5.12 Hz, 1H), 9.13 (d, J=7.01 Hz, 1H); MS (ESP+) 373.19 (M+1).

### EXAMPLE 7

### (4-Amino-benzyl)-{4-[2-(6-methyl-pyridin-2-yl)-imidazo[1,2-a]pyridin-3-yl]-pyrimidin-2-yl}-amine

In a sealed tube, a solution of 3-(2-methanesulfonyl-pyrimidin-4-yl)-2-(6-methyl-pyridin-2-yl)-imidazo[1,2-a]pyridine (0.147 mmol; see Example 2 for its preparation) and 4-aminomethyl-phenylamine (0.443 mmol) in CH₃CN (2 mL) was warmed at reflux overnight. The reaction was then concentrated in vacuo and purified via preparatory HPLC (5→45% CH₃CN/H₂O with 0.1% TFA) to yield 32 mg of (4-amino-benzyl)-{4-[2-(6-methyl-pyridin-2-yl)-imidazo[1,2-a]pyridin-3-yl]-pyrimidin-2-yl}-amine as the TFA salt. ¹H NMR (d₆-DMSO, 400 MHz): 2.58 (s, 3H), 4.58 (d, 2H, J=5.7 Hz), 6.72 (d, 1H, J=5.0 Hz), 7.19 (d, 2H, J=7.2 Hz), 7.42 (d, 2H, J=7.7 Hz), 7.57 (s, 1H), 7.78 (d, 1H, J=7.7 Hz), 7.82 (d, 1H, J=9.3 Hz), 7.92 (d, 1H, 7.2 Hz), 8.16 (s, 1H), 8.29 (d, 1H, J=5.0 Hz); MS (ESP+) 408.2 (M+1).

### EXAMPLE 8

### {4-[2-(6-Methyl-pyridin-2-yl)-imidazo[1,2-a]pyridin-3-yl]-pyrimidin-2-yl}-(2-morpholin-4-yl-ethyl)-amine

In a sealed tube, a solution of 3-(2-methanesulfonyl-pyrimidin-4-yl)-2-(6-methyl-pyridin-2-yl)-imidazo[1,2-a]pyridine (0.147 mmol; see Example 2 for its preparation)) and 2-morpholin-4-yl-ethyl amine (0.443 mmol) in CH₃CN (2 mL) aas warmed atat reflux overnight. The reaction was concentrated in vacuo and purified via preparatory HPLC (5→50% CH₃CN/H₂O with 0.1% TFA) to yield 34 mg of {4-[2-(6-methyl-pyridin-2-yl)-imidazo[1,2-a]pyridin-3-yl]-pyrimidin-2-yl}-(2-morpholin-4-yl-ethyl)-amine as the TFA salt. ¹H NMR (d₆-DMSO, 400 MHz): 2.53 (s, 3H), 3.40 (m, 2H), 3.42 (m, 4H), 3.75 (m, 2H), 3.91 (m, 4H), 6.85 (s, 1H), 7.20(ddd, 1H, J=0.7 Hz, 6.9, 6.9 Hz), 7.39 (d, 1H, J=7.5 Hz), 7.61 (m, 2H), 7.79 (d, 1H, J=7.5 Hz), 7.84 (d, 1H, J=7.5 Hz), 7.91 (dd, 1H, J=7.6, 7.6 Hz), 8.37 (d, 1H, J=4.8 Hz); MS (ESP+) 416.3 (M+1).

### EXAMPLE 9

### N,N-Dimethyl-N′-{4-[2-(6-methyl-pyridin-2-yl)-imidazo[1,2-a]pyridin-3-yl]-pyrimidin-2-yl}-ethane-1,2-diamine

In a sealed tube, a solution of 3-(2-methanesulfonyl-pyrimidin-4-yl)-2-(6-methyl-pyridin-2-yl)-imidazo[1,2-a]pyridine (0.192 mmol; see Example 2 for its preparation) and N,N-dimethylethyldiamine (0.574 mmol) in CH₃CN (2 mL) was warmed at reflux overnight. The reaction was concentrated in vacuo and purified via preparatory HPLC (5→50% CH₃CN/H₂O with 0.1% TFA) to yield 10 mg of N,N-dimethyl-N′-{4-[2-(6-methyl-pyridin-2-yl)-imidazo[1,2-a]pyridin-3-yl]-pyrimidin-2-yl}-ethane-1,2-diamineas the TFA salt. ¹H NMR (d₆-DMSO, 400 MHz): 2.51 (s, 3H), 2.89 (d, 6×3.6 Hz), 3.35 (ddd, 2H, J=6.0, 5.7, 5.7 Hz), 3.72 (ddd, 2H, J=6.0, 5.7 Hz), 6.85 (s, 1H), 7.20 (dd, 1H, J=6.9, 6.9 Hz), 7.40 (d, 1H, 7.8 Hz), 7.61 (m, 2H), 7.80 (d, 1H J=8.0 Hz), 7.84 (d, H, J=8.9 Hz), 7.92 (dd, 1H, J=7.9, 7.9 Hz), 8.36 (d, 1H, J=4.9 Hz); MS (ESP+) 374.2 (M+1).

### EXAMPLE 10

### N-(2-{4-[2-(6-Methyl-pyridin-2-yl)-imidazo[1,2-a]pyridin-3-yl]-pyrimidin-2-ylamino}-ethyl)-acetamide

In a sealed tube, a solution of 3-(2-methanesulfonyl-pyrimidin-4-yl)-2-(6-methyl-pyridin-2-yl)-imidazo[1,2-a]pyridine (0.192 mmol; see Example 2 for its preparation) and N-(2-amino-ethyl)-acetamide (0.689 mmol) in CH₃CN (2 mL) was warmed at reflux overnight. The reaction was concentrated in vacuo and purified via preparatory HPLC (5→50% CH₃CN/H₂O) to yield 22 mg of N-(2-{4-[2-(6-methyl-pyridin-2-yl)-imidazo[1,2-a]pyridin-3-yl]-pyrimidin-2-ylamino}-ethyl)-acetamide. ¹H NMR (d₆-DMSO, 400 MHz): 1.85 (s, 3H), 2.58 (s, 3H), 3.32(q, 2H, J=6.2), 3.46 (bm, 21H) 6.76 (bs, 1H), 7.31 (m, 1H), 7.50 (d, 1H, J=7.7 Hz), 7.72 (dd, 1H, J=8.0 Hz), 7.81 (d, 1H, J=8.0 Hz), 7.90 (d, 1H, J=9.0 Hz), 7.99 (d, 1H, J=8.0 Hz), 8.03 (dd, 1H, J=5.5 Hz), 8.33 (d, 1H, J=5.4 Hz), 9.46 (s, 1H); MS (ESP+) 388.2 (M+1).

### EXAMPLE 11

### N-(4-{4-[2-(6-Methyl-pyridin-2-yl)-imidazo[1,2-a]pyridin-3-yl]-pyrimidin-2-ylamino}-butyl)-acetamide

In a sealed tube, a slurry of 3-(2-methanesulfonyl-pyrimidin-4-yl)-2-(6-methyl-pyridin-2-yl)-imidazo[1,2-a]pyridine (0.171 mmol; see Example 2 for its preparation), N-acetylputricine hydrochlorate (0.546 mmol) and Cs₂CO₃ (0.683 mmol in CH₃CN (2 mL) was warmed at reflux overnight. The reaction was concentrated in vacuo and purified via preparatory HPLC (5→50% CH₃CN/H₂O with 0.1% TFA) to yield 12 mg of N-(4-{4-[2-(6-methyl-pyridin-2-yl)-imidazo[1,2-a]pyridin-3-yl]-pyrimidin-2-ylamino}-butyl)-acetamide as the TFA salt. ¹H NMR (d-DMSO, 400 MHz): 1.51 (quintet, 2H, J=6.7 Hz), 1.51 (quintet, 2H, J=6.7 Hz), 1.82 (s, 3H), 2.58 (s, 3H), 3.10 (q, 2H, 6.5 Hz), 3.39 (bm, 2H), 6.75 (bs, 1H), 7.30 (bm, 1H), 7.49 (d, 1H J=7.1 Hz), 7.71 (dd, 1H, J=7.7 Hz), 7.81 (d, 1H, J=8 Hz), 7.86 (m, 1H), 7.90 (d, 1H, J=9 Hz), 7.99 (dd, 1H, J=8.0, 8.0 Hz), 8.30 (d, 1H, J=5.6 Hz); MS (ESP+) 416.2 (M+1).

### EXAMPLE 12

### {4-[2-(6-Methyl-pyridin-2-yl)-imidazo[1,2-a]pyridin-3-yl]-pyrimidin-2-yl}-(2-pyridin-3-yl-ethyl)-amine

In a sealed tube, a solution of 3-(2-methanesulfonyl-pyrimidin-4-yl)-2-(6-methyl-pyridin-2-yl)-imidazo[1,2-a]pyridine (0.171 mmol; see Example 2 for its preparation) and pyridin-3-yl-ethylamine (0.565 mmol) in CH₃CN (2 mL) was warmed at reflux overnight. The reaction was concentrated in vacuo and purified via preparatory HPLC (5→50% CH₃CN/H₂O with 0.1% TFA) to yield 40 mg of {4-[2-(6-methyl-pyridin-2-yl)-imidazo[1,2-a]pyridin-3-yl]-pyrimidin-2-yl}-(2-pyridin-3-yl-ethyl)-amine as the TFA salt. ¹H NMR (d₆-DMSO, 400 MHz): 2.54 (s, 3H), 3.10 (t, 2H, J=6.5 Hz), 3.71 (m, 2H), 6.76 (bs, 1H), 7.23 (dd, 1H, J=6.8, 6.8 Hz), 7.44 (d, 1H, J=7.8 Hz), 7.65 (dd, 1H, J=7.8 Hz), 7.72 (bm, 1H), 7.78 (d, 1H, J=7.3 Hz), 7.83 (bm, 1H), 7.87 (d, 1H, 9.4 Hz), 7.95 (dd, 1H, J=8.3, 8.3 Hz), 8.31 (d, 1H, J=5.7 Hz), 8.71 (d, 1H, J=6.2 Hz), 8.79 (bs, 1H); MS (ESP+) 408.2 (M+1).

### EXAMPLE 13

### 14-[2-(6-Methyl-pyridin-2-yl)-imidazo[1,2-a]pyridin-3-yl]-pyrimidin-2-yl}-(2-pyridin-4-yl-ethyl)-amine

In a sealed tube, a solution of 3-(2-methanesulfonyl-pyrimidin-4-yl)-2-(6-methyl-pyridin-2-yl)-imidazo[1,2-a]pyridine (0.171 mmol; see Example 2 for its preparation) and pyridin-4-yl-ethylamine (0.565 mmol) in CH₃CN (2 mL) was warmed at reflux overnight. The reaction was concentrated in vacuo and purified via preparatory HPLC (5→50% CH₃CN/H₂O with 0.1% TFA) to yield 67 mg of {4-[2-(6-methyl-pyridin-2-yl)-imidazo[1,2-a]pyridin-3-yl]-pyrimidin-2-yl}-(2-pyridin-4-yl-ethyl)-amine as the TFA salt. ¹H NMR (d₆-DMSO, 400 MHz): 2.53 (s, 3), 3.20 (t, 2H, J=6.9 Hz), 3.76 (q, 2H, J=6.4 Hz), 6.77 (bs, 1H), 7.21 (dd, 1H, J=7.0, 7.0 Hz), 7.42 (d, 1H, 7.6 Hz), 7.63 (dd, 1H, J=8.2, 8.2 Hz), 7.68 (m, 1H), 7.79 (d, 1H, J=7.7 Hz), 7.86 (d, 1H, J=9.0 Hz), 7.90 (m, 1H), 7.94 (dd, 1H, J=7.8, 7.8 Hz), 8.32 (d, 1H, J=5.4 Hz), 8.79 (d, 1H, J=5.3 Hz); MS (ESP+) 408.2 (M+1).

### EXAMPLE 14

### (4-{4-[2-(6-Methyl-pyridin-2-yl)-imidazo[1,2-a]pyridin-3-yl]-pyrimidin-2-ylamino}-butyl)-carbamic acid tert-butyl ester

In a sealed tube, a solution of 3-(2-methanesulfonyl-pyrimidin-4-yl)-2-(6-methyl-pyridin-2-yl)-imidazo[1,2-a]pyridine (0.191 mmol; see Example 2 for its preparation) and (4-amino-butyl)-carbamic acid tert-butyl ester (0.611 mmol) in CH₃CN (2 mL) was warmed at reflux overnight. The reaction was concentrated in vacuo and purified via preparatory HPLC (5→50% CH₃CN/H₂O) to yield 44 mg of (4-{4-[2-(6-methyl-pyridin-2-yl)-imidazo[1,2-a]pyridin-3-yl]-pyrimidin-2-ylamino}-butyl)-carbamic acid tert-butyl ester. ¹H NMR (d₆-DMSO, 400 MHz): 1.39 (s, 9H), 1.50 (quintet, 2H, J=6.8 Hz), 1.58 (quintet, 2H, J=6.8 Hz), 2.53 (s, 3 Hz), 2.98 (q, 2H, J=6.6 Hz), 3.35 (m, 2H), 6.60 (bs, 1H), 6.81 (dd, 1H, J=5.3, 5.3 Hz), 7.09 (dd, 1H, J=6.4, 6.4 Hz), 7.28 (d, 1H, J=7.4 Hz), 7.32 (m, 1H), 7.47 (ddd, 1H, J=1.1, 6.8, 6.8 Hz), 7.73 (dd, 1H, J=7.7, 7.7 Hz), 7.81 (dd, 1H, J=7.7, 7.7 Hz), 8.19 (d, 1H, 5.5 Hz), 9.45 (bs, 1H); MS (ESP+) 474.2 (M+1).

### EXAMPLE 15

### N¹-{4-[2-(6-Methyl-pyridin-2-yl)-imidazo[1,2-a]pyridin-3-yl]-pyrimidin-2-yl}-butane-1,4-diamine

A solution of (4-{4-[2-(6-methyl-pyridin-2-yl)-imidazo[1,2-a]pyridin-3-yl]-pyrimidin-2-ylamino}-butyl)-carbamic acid tert-butyl ester (see Example 14 for its preparation) in 1:1 CH₂Cl₂/TFA (2 mL) was allowed to stir for 30 min, then dried in vacuo to yield 12 mg of N¹-{4-[2-(6-methyl-pyridin-2-yl)-imidazo[1,2-a]pyridin-3-yl]-pyrimidin-2-yl}-butane-1,4-diamine as the tri-TFA salt. ¹H NMR (d-DMSO, 400 MHz): 1.67 (m, 4H), 2.56 (s, 3H), 2.87 (m, 2H), 3.42 (m, 2H), 6.76 (bs, 1H), 7.28 (dd, 1×, J=6.9, 6.9 Hz), 7.47 (d, 1H, J=7.9 Hz), 7.69 (m, 1H), 7.74 (bs, 2H), 7.81 (d, 1H, J=7.8 Hz), 7.90 (d, 1H, J=9.0 Hz), 7.97 (dd, 1H J=7.9, 7.9 Hz), 8.31 (d, 1H, J=5.4 Hz), 9.49 (bs, 1H); MS (ESP+) 374.3 (M+1).

### EXAMPLE 16

### (3-{4-[2-(6-Methyl-pyridin-2-yl)-imidazo[1,2-a]pyridin-3-yl]-pyrimidin-2-ylamino}-propyl)-carbamic acid tert-butyl ester

In a sealed tube, a solution of 3-(2-methanesulfonyl-pyrimidin-4-yl)-2-(6-methyl-pyridin-2-yl)-imidazo[1,2-a]pyridine (0.191 mmol; see Example 2 for its preparation) and (3-amino-propyl)-carbamic acid tert-butyl ester (0.602 mmol) in CH₃CN (2 mL) was warmed at reflux overnight. The reaction was concentrated in vacuo and purified via preparatory HPLC (5→50% CH₃CN/H₂O) to yield 27 mg of (3-{4-[2-(6-methyl-pyridin-2-yl)-imidazo[1,2-a]pyridin-3-yl]-pyrimidin-2-ylamino}-propyl)-carbamic acid tert-butyl ester. ¹H NMR (d₆-DMSO, 400 MHz): 1.40 (s, 9H), 1.73 (quintet, 2H, J=6.7 Hz), 2.53 (s, 3H), 3.05 (q, 2H, J=6.2 Hz), 3.36 (m, 2H), 6.63 (bs, 1H), 6.87 (dd, 1H, J=5.6, 5.6 Hz), 7.12 (dd, 1H, 6.5, 6.5 Hz), 7.30 (m, 1H), 7.31 (d, 1H, J=7.7 Hz), 7.51 (ddd, 1H, J=1.1, 6.8, 6.8 Hz), 7.77 (dd, 2H, 7.7 Hz), 7.84 (dd, 1H, J=7.1, 7.1 Hz), 8.23 (d, 1H, J=5.3 Hz), 9.47 (bs, 1H); MS (ESP+) 459.9 (M+1).

### EXAMPLE 17

### N¹-{4-[2-(6-Methyl-pyridin-2-yl)-imidazo[1,2-a]pyridin-3-yl]-pyrimidin-2-yl}-propane-1,3-diamine

A solution of (3-{4-[2-(6-methyl-pyridin-2-yl)-imidazo[1,2-a]pyridin-3-yl]-pyrimidin-2-ylamino}-propyl)-carbamic acid tert-butyl ester (see Example 16 for its preparation) in 1:1 CH₂Cl₂/TFA (2 mL) was allowed to stir at RT for 30 min, then dried in vacuo to yield 48 mg of N′-{4-[2-(6-methyl-pyridin-2-yl)-imidazo[1,2-a]pyridin-3-yl]-pyrimidin-2-yl}-propane-1,3-diamine as the tri-TFA salt. ¹H (d₆-DMSO, 400 MHz): 1.90 (quintet, 2H, J=7.8 Hz), 2.55 (s, 3H), 2.93 (septet, 2H, J a 6.7 Hz), 3.45 (q, 2H, J=5.4 Hz), 6.76 (bs, M1), 7.23 (dd, 1H, J=7.0, 7.0 Hz), 7.43 (d, 1H, J=7.4 Hz), 7.67 (m, 41), 7.80 (d, 1H, J=7.9 Hz), 7.86 (d, 111 J=9.1 Hz), 7.95 (dd, 1H, J=7.9, 7.9 Hz), 8.32 (d, 1H, J=5.3 Hz), 9.44 (bs, 1H); MS (ESP+) 360.2 (M+1).

### EXAMPLE 18

### {4-[2-(6-Methyl-pyridin-2-yl)-imidazo[1,2-a]pyridin-3-yl]-pyrimidin-2-yl}-(3-morpholin-4-yl-propyl)-amine

In a sealed tube, a solution of 3-(2-methanesulfonyl-pyrimidin-4-yl)-2-(6-methyl-pyridin-2-yl)-imidazo[1,2-a]pyridine (0.205 mmol; see Example 2 for its preparation) and 3-propylamino morpholine (0.670 mmol) in CH₃CN (2 mL) was warmed at reflux overnight. The reaction was concentrated in vacuo and purified via preparatory HPLC (5→50% CH₃CN/H₂O with 0.1% TFA) to yield 20 mg of {4-[2-(6-methyl-pyridin-2-yl)-imidazo[1,2-a]pyridin-3-yl]-pyrimidin-2-yl}-(3-morpholin-4-yl-propyl)-amine as the TFA salt. ¹H NMR (d₆-DMSO, 400 MHz): 1.97 (m, 2H), 2.51 (s, 3H), 3.08 (m, 2H), 3.18 (m, 2H), 3.41 (m, 4H), 3.64 (bs, 2H), 3.94 (bs, 2H), 6.73 (bs, 1H), 7.17 (dd, 1H, J=6.5, 6.5 Hz), 7.37 (d, 1H, J=7.8 Hz), 7.58 (m, 1H), 7.61 (m, 1H), 7.76 (d, 1H, J=8.0 Hz), 7.81 (d, 1H, J=9.0 Hz), 7.89 (dd, 1H, J=7.6, 7.6 Hz), 8.28 (d, 1H, J=5.4 Hz), 9.39 (bs, 1H); MS (ESP+) 430.2 (M+1).

### EXAMPLE 19

### {4-[2-(6-Methyl-pyridin-2-yl)-imidazo[1,2-a]pyridin-3-yl]-pyrimidin-2-yl}-(2-pyridin-2-yl-ethyl)-amine

In a sealed tube, a solution of 3-(2-methanesulfonyl-pyrimidin-4-yl)-2-(6-methyl-pyridin-2-yl)-imidazo[1,2-a]pyridine (0.193 mmol; see Example 2 for its preparation) and 2-pyridin-2-yl-ethylamine (0.641 mmol) in CH₃CN (2 mL) was warmed at reflux overnight. The reaction concentrated in vacuo and purified via preparatory HPLC (5→50% CH₃CN/H₂O) to yield 24 mg of {4-[2-(6-methyl-pyridin-2-yl)-imidazo[1,2-a]pyridin-3-yl]-pyrimidin-2-yl)-(2-pyridin-2-yl-ethyl)-amine. ¹H NMR (CDCl₃, 400 MHz): 2.43 (s, 3H), 3.06 (t, 2H, J=6.7 Hz), 3.71 (q, 2H, J=6 Hz), 6.63 (d, 1H, J=5.0 Hz), 7.07 (ddd, 1H, J=0.8, 7.1, 7.1 Hz), 7.23 (m, 1H), 7.28 (m, 2H), 7.48 (m, 1H), 7.72 (m, 21), 7.81 (t, 1H, 7.9 Hz), 8.20 (d, 1H, J=5.4 Hz), 8.53 (dddd, 1H, J=0.8, 5.0, 5.0, 5.0 Hz), 9.47 (d, 1, J=7.1 Hz); MS (ESP+) 408.1 (M+1).

### EXAMPLE 20

### 4-(2-{4-[2-(6-Methyl-pyridin-2-yl)-imidazo[1,2-a]pyridin-3-yl]-pyrimidin-2-ylamino}-ethyl)-benzenesulfonamide

In a sealed tube, a solution of 3-(2-methanesulfonyl-pyrimidin-4-yl)-2-(6-methyl-pyridin-2-yl)-imidazo[1,2-a]pyridine (0.193 mmol; see Example 2 for its preparation) and 4-(2-amino-ethyl)-benzenesulfonamide (0.639 mmol) in DMF (2 mL) was warmed at reflux overnight. The reaction was concentrated in vacuo and purified via preparatory HPLC (5→50% CH₃CN/H₂O) to yield 13 mg of 4-(2-{4-[2-(6-methyl-pyridin-2-yl)-imidazo[1,2-a]pyridin-3-yl]-pyrimidin-2-ylamino}-ethyl)-benzenesulfonamide. ¹H NMR (d₆-DMSO, 400 MHz): 2.43 (s, 3H), 2.98 (t, 2H, J=6.5 Hz), 3.59 (q, 2H, J=6.5 Hz), 6.66 (bs, 1H), 7.07 (dd, 1H, J=7.0, 7.0 Hz), 7.25 (m, 3H), 7.48 (m, 4H), 7.75 (m, 4H), 7.81 (dd, 1H, J=7.7, 7.7 Hz), 8.23 (d, 1H, J=5.0 Hz), 9.40 (bs, 1H); MS (ESP+) 486.0 (M+1).

### EXAMPLE 21

### Methyl-{4-[2-(6-methyl-pyridin-2-yl)-imidazo[1,2-a]pyridin-3-yl]-pyrimidin-2-yl}-amine

In a sealed tube, a solution of 3-(2-methanesulfonyl-pyrimidin-4-yl)-2-(6-methyl-pyridin-2-yl)-imidazo[1,2-a]pyridine (0.137 mmol; see Example 2 for its preparation) and 2.00 M methylamine/THF (1.00 mmol) in CH₃CN (2 mL) was warmed at reflux overnight. The reaction was concentrated in vacuo and purified via preparatory HPLC (5→50% CH₃CN/) to yield 17 mg of methyl-{4-[2-(6-methyl-pyridin-2-yl)-imidazo[1,2-a]pyridin-3-yl]-pyrimidin-2-yl}-amine. ¹H NMR (d₆-DMSO, 400 MHz): 2.46 (s, 3H), 2.93 (d, 3H, J=5.0 Hz), 6.66 (d, 1H, J=5.4 Hz), 7.12 (dd, 1H J=6.7, 6.7 Hz), 7.28 (m, 1H), 7.32 (d, 1H, J=7.8 Hz), 7.51 (m, 1H), 7.78 (m, 2H), 7.85 (dd, 1H, J=7.6, 7.6 Hz), 8.25 (d, 1H, J=5.0 Hz), 9.49 (d, 1H, J=7.0 Hz); MS (ESP+) 317.2 (M+1).

### EXAMPLE 22

### Dimethyl-{4-[2-(6-methyl-pyridin-2-yl)-imidazo[1,2-a]pyridin-3-yl]-pyrimidin-2-yl}-amine

In a sealed tube, a solution of 3-(2-methanesulfonyl-pyrimidin-4-yl)-2-(6-methyl-pyridin-2-yl)-imidazo[1,2-a]pyridine (0.136 mmol; see Example 2 for its preparation) and 2.0 M dimethylamine/THF (1 mmol) in CH₃CN (2 mL) was warmed at reflux overnight. The reaction was concentrated in vacuo and purified via preparatory HPLC (5→50% CH₃CN/H₂O) to yield 17 mg of dimethyl-{4-[2-(6-methyl-pyridin-2-yl)-imidazo[1,2-a]pyridin-3-yl]-pyrimidin-2-yl}-amine. ¹H NMR (d₆-DMSO, 400 MHz): 2.46 (s, 3H), 3.24 (s, 6H), 6.70 (d, 1H, J=5.0 Hz), 7.14 (dd, 1H, J=6.8, 6.8 Hz), 7.32 (d, 1H, J=7.3 Hz), 7.52 (dd, 1H, J=8.0, 8.0 Hz), 7.79 (m, 2H), 7.85 (d, 1H, J=7.8, 7.8 Hz), 8.33 (d, 1H, J=5.4, 5.4 Hz), 9.39 (d, 1H, J=7.2 Hz); MS (ESP+) 331.2 (M+1).

### EXAMPLE 23

### (3-Imidazol-1-yl-propyl)-{4-[2-(6-methyl-pyridin-2-yl)-imidazo[1,2-a]pyridin-3-yl]-pyrimidin-2-yl}-amine

In a sealed tube, a solution 3-(2-methanesulfonyl-pyrimidin-4-yl)-2-(6-methyl-pyridin-2-yl)-imidazo[1,2-a]pyridine (0.273 mmol; see Example 2 for its preparation) and imidazol-1-yl-propylamine (0.905 mmol) in CH₃CN (4 mL) was warmed at reflux overnight. The reaction was concentrated in vacuo and purified via preparatory HPLC (5→50% CH₃CN/H₂O with 0.1% TFA) to yield 8 mg of (3-imidazol-1-yl-propyl)-{4-[2-(6-methyl-pyridin-2-yl)-imidazo[1,2-a]pyridin-3-yl]-pyrimidin-2-yl}-amine as the TFA salt. ¹H NMR (d₆-DMSO, 400 MHz): 2.12 (p, 2H, J=6.8 Hz), 2.45 (s, 3H), 3.37 (m, 2H), 4.23 (t, 2H, J=7.1 Hz), 6.70 (bs, 1H), 7.11 (dd, 1H, J=6.9, 6.9 Hz), 7.31 (d, 1H, J=7.6), 7.34 (bs, 1H), 7.50 (m, 2H), 7.57 (bs, 1H), 7.78 (m, 2H), 7.85 (dd, 1H, J=8.0, 8.0 Hz), 8.26 (d, 1H, 5.3 Hz), 8.46 (bs, 1H), 9.38 (bs, 1H); MS (ESP+) 411.2 (M+1).

### EXAMPLE 24

### 3-{4-[2-(6-Methyl-pyridin-2-yl)-imidazo[1,2-a]pyridin-3-yl]-pyrimidin-2-ylamino}-phenol

In a sealed tube, a slurry of solid KIDS (0.501 mmol) and 3-aminophenol (0.458 mmol) in anhydrous dioxane (2 mL) was stirred for 1 hour. 3-(2-Methanesulfonyl-pyrimidin-4-yl)-2-(6-methyl-pyridin-2-yl)-imidazo[1,2-a]pyridine (0.137 mmol; see Example 2 for its preparation) was added to the reaction and the mixture was allowed to stir for 2 days at 80° C. The mixture was concentrated in vacuo and purified via preparatory HPLC (5→50% CH₃CN/H₂O with 0.1% TFA) to yield 12 mg of 3-{4-[2-(6-methyl-pyridin-2-yl)-imidazo[1,2-a]pyridin-3-yl]-pyrimidin-2-ylamino}-phenol as the TFA salt. ¹H NMR (d₆-DMSO, 400 MHz): 2.55 (s, 3H), 6.70 (m, 2H), 7.02 (dd, 1H, J=7.1, 7.1 Hz Hz), 7.28 (m, 1H), 7.40 (d, 1H, J=5.3 Hz), 7.44 (d, 1H, J=7.3 Hz), 7.58 (dd, 1H, J=7.6, 7.6 Hz), 7.83 (m, 2H), 7.95 (dd, 1H, J=7.3, 7.3 Hz), 8.62 (d, 1H, J=5.0 Hz), 9.13 (d, 1H, J=6.5 Hz); MS (ESP+) 395.2 (M+1).

### EXAMPLE 25

### {4-[2-(6-Methyl-pyridin-2-yl)-imidazo[1,2-a]pyridin-3-yl]-pyrimidin-2-yl}-[1,3,4]thiadiazol-2-yl-amine

In a sealed tube, a slurry of solid KHMDS (0.501 mmol) and [1,3,4]thiadiazol-2-ylamine (0.454 mmol) in anhydrous dioxane (2 mL) was stirred for 1 hour. 3-(2-Methanesulfonyl-pyrimidin-4-yl)-2-(6-methyl-pyridin-2-yl)-imidazo[1,2-a]pyridine (0.137 mmol; see Example 2 for its preparation) was added to the reaction and the mixture was allowed to stir for 2 days at 80° C. The mixture was concentrated in vacuo and purified via preparatory HPLC (5→50% CH₃CN/H₂O with 0.1% TFA) to yield 23 mg of {4-[2-(6-methyl-pyridin-2-yl)-imidazo[1,2-a]pyridin-3-yl]-pyrimidin-2-yl}-[1,3,4]thiadiazol-2-yl-amine as the TFA salt. ¹H NMR (d₆-DMSO, 400 MHz): 2.51 (s, 3H), 7.17 (m, 2H), 7.37 (d, 1H, 7.4 Hz), 7.60 (dd, 1H, J=7.5, 7.5 Hz), 7.86 (m, 3H), 8.61 (d, 1H, J=5.3 Hz), 9.07 (s, 1H), 9.59 (bs, 1H); MS (ESP+) 387.1 (M+1).

### EXAMPLE 26

### (5-{4-[2-(6-Methyl-pyridin-2-yl)-imidazo[1,2-a]pyridin-3-yl]-pyrimidin-2-ylamino}-pentyl)-carbamic acid tert-butyl ester

In a sealed tube, a solution of 3-(2-methanesulfonyl-pyrimidin-4-yl)-2-(6-methyl-pyridin-2-yl)-imidazo[1,2-a]pyridine (0.205 mmol; see Example 2 for its preparation) and (5-amino-pentyl)-carbamic acid tert-butyl ester (0.677 mmol) in acetonitrile (2 mL) was warmed at reflux overnight. The reaction was concentrated in vacuo and purified via preparatory HPLC (5→50% CH₃CN/H₂O with 0.1% TFA) to yield 65 mg of (5-{4-[2-(6-methyl-pyridin-2-yl)-imidazo[1,2-a]pyridin-3-yl]-pyrimidin-2-ylamino}-pentyl)-carbamic acid tert-butyl ester as the TFA salt. ¹H NMR (d₆-DMSO, 400 MHz): 1.36 (s, 9H), 1.40 (m, 2H), 1.62 (m, 2H), 2.58 (s, 3H), 2.96 (m, 2H), 3.37 (m, 2H), 6.78 (bs, 1H), 6.82 (bs, 1H), 7.30 (dd, 1H, J=5.0, 5.0 Hz), 7.49 (d, 1H, J=7.5 Hz), 7.70 (dd, 1H, J=8.1, 8.1 Hz), 7.81 (d, 1H, J=7.5 Hz), 7.90 (d, 1H, J=8.1 Hz), 7.95 (dd, 1H, J=7.5, 7.5 Hz), 8.30 (d, 1H, J=5.0 Hz); MS (ESP+) 488.2 (M+1).

### EXAMPLE 27

### [3-(4-Methyl-piperazin-1-yl)-propyl]-{4-[2-(6-methyl-pyridin-2-yl)-imidazo[1,2-a]pyridin-3-yl]-pyrimidin-2-yl}-amine

In a sealed tube, a solution of 3-(2-methanesulfonyl-pyrimidin-4-yl)-2-(6-methyl-pyridin-2-yl)-imidazo[1,2-a]pyridine (0.205 mmol; see Example 2 for its preparation) and 3-(4-methyl-piperazin-1-yl)-propylamine (0.680 mmol) in acetonitrile (2 mL) was warmed to reflux overnight. The mixture was concentrated it vacuo and purified on the preparatory HPLC (5→50% CH₃CN/H₂O) to yield 20 mg of [3-(4-methyl-piperazin-1-yl)-propyl]-{4-[2-(6-methyl-pyridin-2-yl)-imidazo[1,2-a]pyridin-3-yl]-pyrimidin-2-yl}-amine. ¹H NMR (d₆-DMSO, 400 MHz): 1.78 (m, 2H), 2.28 (s, 3H), 2.46 (s, 3H), 3.38 (m, 2H), 6.65 (bs, 1H), 7.11 (ddd, 1H, J=1.0, 7.0, 7.0 Hz), 7.32 (d, 1H, J=7.3 Hz), 7.40 (s, 1H), 7.51 (m, 1H, J=7.8 Hz), 7.78 (m, 2H), 7.85 (dd, 1H, J=7.8, 7.8 Hz), 8.24 (d, 1H, J=5.5 Hz), 9.45 (bs, 1H); MS (ESP+) 443.4 (M+1).

### EXAMPLE 28

### Cyclopropyl-{4-[2-(6-methyl-pyridin-2-yl)-imidazo[1,2-a]pyridin-3-yl]-pyrimidin-2-yl}-amine

In a sealed tube, a solution of 3-(2-methanesulfonyl-pyrimidin-4-yl)-2-(6-methyl-pyridin-2-yl)-imidazo[1,2-a]pyridine (0.205 mmol; see Example 2 for its preparation) and cyclopropylamine (14.4 mmol) in acetonitrile (2 mL) was warmed to reflux overnight. The mixture was concentrated in vacuo and purified via preparatory HPLC (5→50% CH₃CN/H₂O with 0.1% TFA) to yield 40 mg of cyclopropyl-{4-[2-(6-methyl-pyridin-2-yl)-imidazo[1,2-a]pyridin-3-yl]-pyrimidin-2-yl}-amine as the TFA salt. ¹H NMR (d₆-DMSO, 400 MHz): 0.66 (m, 2H), 0.84 (m, 2H), 2.58 (s, 3H), 2.82 (m, 1H), 6.82 (d, 2H, J=5.5 Hz), 7.30 (dd, 1H, J=7.5, 7.5 Hz), 7.48 (d, 1H, J=7.9 Hz), 7.70 (dd, 1H, J=8.1, 8.1 Hz), 7.83 (d, 1H, J=7.5 Hz), 7.89 (d, 1H, 9.3 Hz), 7.98 (dd, 1H, J=8.0, 8.0 Hz), 8.30 (d, 1H, J=5.6 Hz), 9.74 (bs, 1H); MS (ESP+) 343.3 (M+1).

### EXAMPLE 29

### 2-(6-Methyl-pyridin-2-yl)-3-(2-pyrrolidin-1-yl-pyrimidin-4-yl)-imidazo[1,2-a]pyridine

In a sealed tube, a solution of 3-(2-methanesulfonyl-pyrimidin-4-yl)-2-(6-methyl-pyridin-2-yl)-imidazo[1,2-a]pyridine (0.205 mmol; see Example 2 for its preparation) and pyrrolidine (1.20 mmol) in acetonitrile (2 mL) was warmed to reflux overnight. The mixture was concentrated in vacuo and purified via preparatory HPLC (5→80% CH₃CN/H₂O with 0.1% TFA) to yield 40 mg 2-(6-methyl-pyridin-2-yl)-3-(2-pyrrolidin-1-yl-pyrimidin-4-yl)-imidazo[1,2-a]pyridine as the TFA salt. ¹H NMR (d₆-DMSO, 400 MHz): 2.00 (bm, 4H), 2.54 (s, 3H), 3.60 (bm, 4H), 6.71 (bs, 1H), 7.26 (m, 1H), 7.44 (d, 1H, 7.3 Hz), 7.68 (bm, 1H), 7.77 (d, 1H, J=7.9 Hz), 7.86 (d, 1H, J=8.9 Hz), 7.95 (bm, 1H), 8.33 (d, 1H, J=5.4 Hz), 9.46 (d, 1H, J=6.1 Hz); MS (ESP+) 357.4 (N+1).

### EXAMPLE 30

### 2-(6-Methyl-pyridin-2-yl)-3-(2-piperidin-1-yl-pyrimidin-4-yl)-imidazo[1,2-a]pyridine

In a sealed tube, a solution of 3-(2-methanesulfonyl-pyrimidin-4-yl)-2-(6-methyl-pyridin-2-yl)-imidazo[1,2-a]pyridine (0.205 mmol; see Example 2 for its preparation) and piperidine (1.01 mmol) in acetonitrile (2 mL) was warmed to reflux overnight. The mixture was concentrated in vacuo and purified via preparatory HPLC (5→80% CH₃CN/H₂O with 0.1% TFA) to yield 24 mg of 2-(6-methyl-pyridin-2-yl)-3-(2-piperidin-1-yl-pyrimidin-4-yl)-imidazo[1,2-a]pyridine as the TFA salt. ¹H NMR (d₆-DMSO, 400 MHz): 1.59 (m, 4H), 1.67 (m, 2H), 2.55 (s, 3H), 3.81 (m, 4H), 6.74 (d, 1H, J=5.0 Hz), 7.24 (ddd, 1H, J=0.8, 7.7, 7.7 Hz), 7.43 (d, 1H, 7.6 Hz), 7.63 (m, 1H), 7.77 (d, 1H, J=7.7 Hz), 7.84 (d, 1H, J=9.2 Hz), 7.92 (t, 1H, J=7.9 Hz), 8.37 (d, 1H, J=5.2 Hz), 9.18 (d, 1H, J=7.1 Hz); MS (ESP+) 371.4 (M+1).

### EXAMPLE 31

### (2-Methoxy-ethyl)-{4-[2-(6-methyl-pyridin-2-yl)-imidazo[1,2-a]pyridin-3-yl]-pyrimidin-2-yl}-amine

In a sealed tube, a solution of 3-(2-methanesulfonyl-pyrimidin-4-yl)-2-(6-methyl-pyridin-2-yl)-imidazo[1,2-a]pyridine (0.205 mmol; see Example 2 for its preparation) and 2-methoxy-ethylamine (1.16 mmol) in acetonitrile (2 mL) was warmed to reflux overnight. The mixture was concentrated in vacuo and purified via preparatory HPLC (5→80% CH₃CN/H₂O with 0.1% TFA) to yield 42 mg of(2-methoxy-ethyl)-4-[2-(6-methyl-pyridin-2-yl)-imidazo[1,2-a]pyridin-3-yl]-pyrimidin-2-yl}-amine as the TFA salt. ¹H NMR (d-DMSO, 400 MHz): 2.58 (s, 3H), 3.34 (s, 3H), 3.57 (bm, 4H), 6.77 (bs, 1H), 7.28 (dd, 1H, J=7.1, 7.1 Hz), 7.49 (d, 1H, J=8 Hz), 7.78 (dd, 1H, J=8.7, 8.7 Hz), 7.82 (d, 1H J=7.7 Hz), 7.89 (d, 1H, J=9.3 Hz), 7.99 (dd, 1H, J=7.8, 7.8 Hz), 8.31 (d, 1H, J=5.5 Hz), 9.50 (bs, 1H); MS (ESP+) 361.4 (M+1).

### EXAMPLE 32

### 3-[2-(4-Methyl-piperazin-1-yl)-pyrimidin-4-yl]-2-(6-methyl-pyridin-2-yl)-imidazo[1,2-a]pyridine

In a sealed tube, a solution of 3-(2-methanesulfonyl-pyrimidin-4-yl)-2-(6-methyl-pyridin-2-yl)-imidazo[1,2-a]pyridine (0.205 mmol; see Example 2 for its preparation) and 1-methyl-piperazine (0.900 mmol) in acetonitrile (2 mL) was warmed to reflux ovenight. The mixture was concentrated in vacuo and purified via preparatory HPLC (5→80% CH₃CN/H₂O with 0.1% TFA) to yield 19 mg of 3-[2-(4-methyl-piperazin-1-yl)-pyrimidin-4-yl]-2-(6-methyl-pyridin-2-yl)-imidazo[1,2-a]pyridine as the TFA salt. ¹H NMR (4-DMSO, 400 MHz): 2.45 (s, 3H), 2.89 (s, 3H), 3.13 (m, 2H), 3.33 (m, 2H), 3.57 (m, 2H), 4.74 (m, 2H), 6.94 (d, 1H, J=5.1 Hz), 7.15 (dd, 1H, J=7.0, 7.0 Hz), 7.35 (d, 1H, J=7.9 Hz), 7.57 (dd, 1H, J=7.7, 7.7 Hz), 7.80 (d, 1H, J=8.2 Hz), 7.85 (d, 1H, J=9.6 Hz), 7.89 (dd, 1H, J=7.9, 7.9 Hz), 8.46 (d, 1H, J=5.5 Hz), 9.16 (d, 1H, J=7.0 Hz), 9.93 (bs, 1H); MS (ESP+) 386.4 (M+1).

### EXAMPLE 33

### 3-[2-(2-Methyl-aziridin-1-yl)-pyrimidin-4-yl]-2-(6-methyl-pyridin-2-yl)-imidazo[1,2-a]pyridine

In a sealed tube, a solution of 3-(2-methanesulfonyl-pyrimidin-4-yl)-2-(6-methyl-pyridin-2-yl)-imidazo[1,2-a]pyridine (0.137 mmol; see Example 2 for its preparation) and 2-methyl-aziridine (0.849 mmol) in acetonitrile (2 mL) was warmed to reflux overnight. The mixture was concentrated in vacuo and purified via preparatory HPLC (5→50% CH₃CN/H₂O with 0.1% TFA) to yield 11 mg of 3-[2-(2-methyl-aziridin-1-yl)-pyrimidin-4-yl]-2-(6-methyl-pyridin-2-yl)-imidazo[1,2-a]pyridine as the TFA salt. ¹H NMR (d₆-DMSO, 400 MHz): 1.16 (d, 3H, J=5.8 Hz), 2.56 (s, 3H), 3.36 (m, 2H), 3.90 (m, 1H), 6.75 (m, 1H), 7.16 (m, 1H), 7.45 (m, 1H), 7.72 (m, 2H), 7.91 (m, 1H), 8.20 (m, 1H), 9.54 (m, 1H); MS (ESP+) 343.2 (M+1).

### EXAMPLE 34

### {4-[2-(6-Methyl-pyridin-2-yl)-imidazo[1,2-a]pyridin-3-yl]-pyrimidin-2-yl}-(2H-pyrazol-3-yl)-amine

In a sealed tube, a slurry of solid KHMDS (0.501 mmol) and 2H-pyrazol-3-ylamine (0.454 mmol) in anhydrous dioxane (2 mL) was stirred for 1 hour. 3-(2-Methanesulfonyl-pyrimidin-4-yl)-2-(6-methyl-pyridin-2-yl)-imidazo[1,2-a]pyridine (0.137 mmol; see Example 2 for its preparation) was added to the reaction and the mixture was allowed to stir for 2 days at 80° C. The mixture was passed through a silica plug, concentrated in vacuo and purified via preparatory HPLC (5→80% CH₃CN/H₂O with 0.1% TFA) to yield 21 mg of {4-[2-(6-methyl-pyridin-2-yl)-imidazo[1,2-a]pyridin-3-yl]-pyrimidin-2-yl}-(2H-pyrazol-3-yl)-amine as the TFA salt. ¹H NMR (d₆-DMSO, 400 MHz): 2.47 (s, 3H), 5.94 (d, 1H, J=2.7 Hz), 7.22 (dd, 1H, J=7.0, 7.0 Hz), 7.39 (m, 1H), 7.61 (dd, 1H, J=8.4, 8.4 Hz), 7.90 (m, 3H), 8.36 (d, 1H, J=2.2 Hz), 8.64 (d, 1H, J=4.9 Hz), 9.56 (d, 1H, J=7.5 Hz); MS (ESP+) 369.2 (M+1).

### EXAMPLE 35

### 2-(6-Methyl-pyridin-2-yl)-3-[2-(1H-tetrazol-5-yl)-pyrimidin-4-yl]-imidazo[1,2-a]pyridine

A solution of 3-(2-methanesulfonyl-pyrimidin-4-yl)-2-(6-methyl-pyridin-2-yl)-imidazo[1,2-a]pyridine (0.200 mmol; see Ex. 2 for its preparation) and sodium cyanide (0.671 mol) in anhydrous DMF (2 mL) was warmed to 100° C. in a sealed tube for 4.5 h. After cooling to RT, the solution was filtered through a Celite pad and concentrated in vacuo to give a dark solid. The solid was slurried in DMSO/water, cooled to 0° C., filtered, washed with cold water and air dried to give 0.0274 g of a tan solid identified as 4-[2-(6-methyl-pyridin-2-yl)-imidazo[1,2-a]pyridin-3-yl]-pyrimidine-2-carbonitrile. MS (ESP+) 313.20 (M+1).

In a sealed tube, 4-[2-(6-methyl-pyridin-2-yl)-imidazo[1,2-a]pyridin-3-yl]-pyrimidine-2-carbonitrile (0.240 mmol), NaN₃ (1.72 mmol), and NH₄Cl (1.27 mmol) were added sequentially to DMF (3 mL). The mixture was heated to 110° C. over two days. The reaction was concentrated in vacuo and purified via preparatory HPLC (CH₃CN/H₂O gradient) to yield 50 mg of 2-(6-methyl-pyridin-2-yl)-3-[2-(1H-tetrazol-5-yl)-pyrimidin-4-yl]-imidazo[1,2-a]pyridine. ¹H NMR (CDCl₃, 400 MHz): 2.48 (s, 3H), 7.22 (dt, 1H, J=1.1, 6.6 Hz), 7.33 (m, 2H), 7.57 (m, 2H), 7.84 (d, 1H, J=8.7 Hz), 7.91 (m, 2H), 8.78 (d, 1H, J=5.6 Hz), 9.87 (d, 1H, J=7.4 Hz); MS (ESP−) 354.1 (M−1).

### EXAMPLE 36

### 3-(2-Azetidin-1-yl-pyrimidin-4-yl)-2-(6-methyl-pyridin-2-yl)-imidazo[1,2-a]pyridine

In a sealed tube, a solution of 3-(2-methanesulfonyl-pyrimidin-4-yl)-2-(6-methyl-pyridin-2-yl)-imidazo[1,2-a]pyridine (0.147 mmol; see Example 2 for its preparation) and azeridine (0.297 mmol) in CH₃CN (2 mL) was allowed to stir at reflux overnight. The reaction was concentrated in vacuo and purified via preparatory HPLC (5→90% CH₃CN/H₂O with 0.1% TFA) to yield 18 mg of 3-(2-azetidin-1-yl-pyrimidin-4-yl)-2-(6-methyl-pyridin-2-yl)-imidazo[1,2-a]pyridine as the TFA salt. ¹H NMR (d₆-DMSO, 400 MHz): 2.43 (quint, 2H, J=7.2 Hz), 2.54 (s, 1H), 4.20 (t, 4H, J=7.5 Hz), 6.81 (d, 1H, J=5.3 Hz), 7.25 (dd, 2H, J=6.9, 6.9 Hz), 7.44 (d, 1H, J=7.9 Hz), 7.65 (dd, 1H, J=7.4, 8.4 Hz), 7.80 (d, 1H, J=7.7 Hz), 7.86 (d, 1H, J=9.0 Hz), 7.95 (dd, 1H, J=7.7, 7.9 Hz), 8.35 (d, 1H, J=5.2 Hz), 9.41 (d, 1H, J=8.9 Hz); MS (ESP+) 343.2 (M+1).

### EXAMPLE 37

### Cyclopentyl-{4-[2-(6-methyl-pyridin-2-yl)-imidazo[1,2-a]pyridin-3-yl]-pyrimidin-2-yl}-amine

In a sealed tube, a solution of 3-(2-methanesulfonyl-pyrimidin-4-yl)-2-(6-methyl-pyridin-2-yl)-imidazo[1,2-a]pyridine (0.137 mmol; see Example 2 for its preparation) and cyclopentylamine (0.452 mmol) in CH₃CN (2 mL) was allowed to stir at reflux overnight. The reaction was concentrated in vacuo and purified via preparatory HPLC (5→50% CH₃CN/H₂O with 0.1% TFA) to yield 11 mg of cyclopentyl-{4-[2-(6-methyl-pyridin-2-yl)-imidazo[1,2-a]pyridin-3-yl]-pyrimidin-2-yl}-amine as the TFA salt. ¹H NMR (d₆-DMSO, 400 MHz): 1.62 (m, 4H), 1.71 (m, 2H), 1.95 (m, 2H), 2.54 (s, 3H), 4.19 (m, 2H), 6.71 (d, 1H, J=4.8 Hz), 7.26 (dd, 1H, J=7.1, 7.1 Hz), 7.44 (d, 1H, J=7.4 Hz), 7.65 (m, 1H), 7.77 (d, 1H, J=7.8 Hz), 7.86 (d, 1H, J=9.0 Hz), 7.94 (dd, 1H, J=7.9, 7.9 Hz), 8.25 (d, 1H, J=5.4 Hz), 9.50 (bs, 1H); MS (ESP+) 371.1 (M+1).

### EXAMPLE 38

### {4-[2-(6-Methyl-pyridin-2-yl)-imidazo[1,2-a]pyridin-3-yl]-pyrimidin-2-yl}-((S)-1-phenyl-ethyl)-amine

In a sealed tube, a solution of 3-(2-methanesulfonyl-pyrimidin-4-yl)-2-(6-methyl-pyridin-2-yl)-imidazo[1,2-a]pyridine (0.137 mmol; see Example 2 for its preparation) and (S)-benzylmethylamine (1.37 mmol) in CH₃CN (2 mL) was allowed to stir at reflux overnight. The reaction was concentrated in vacuo and purified via preparatory HPLC (5→50% CH₃CN/H₂O with 0.1% TFA) to yield 11 mg of {4-[2-(6-methyl-pyridin-2-yl)-imidazo[1,2-a]pyridin-3-yl]-pyrimidin-2-yl}-((S)-1-phenyl-ethyl)-amine as the TFA salt. ¹H NMR (d₆-DMSO, 400 MHz): 1.54 (d, 3H, J=7.0 Hz), 2.56 (s, 3H), 5.09 (bs, 1H), 6.68 (d, 1H, J=5.2 Hz), 7.31 (m, 1H), 7.44 (m, 6H), 7.65 (m, 1H), 7.77 (d, 1H, J=7.3 Hz), 7.85 (d, 1H, J=7.3 Hz), 7.96 (dd, 1H, J=8.3, 7.3 Hz), 8.23 (m, 1H), 8.30 (d, 1H, J=5.0 Hz); MS (ESP+) 407.2 (M+1).

### EXAMPLE 39

### {4-[2-(6-Methyl-pyridin-2-yl)-imidazo[1,2-a]pyridin-3-yl]-pyrimidin-2-yl}-((R)-1-phenyl-ethyl)-amine

In a sealed tube, a solution of 3-(2-methanesulfonyl-pyrimidin-4-yl)-2-(6-methyl-pyridin-2-yl)-imidazo[1,2-a]pyridine (0.137 mmol; see Example 2 for its preparation) and (R)-benzylmethylamine (1.37 mmol) in CH₃CN (2 mL) allowed to stir at reflux overnight. The reaction was concentrated in vacuo and purified via preparatory HPLC (5→50% CH₃CN/H₂O with 0.1% TFA) to yield 11 mg of {4-[2-(6-methyl-pyridin-2-yl)-imidazo[1,2-a]pyridin-3-yl]-pyrimidin-2-yl}-((S)-1-phenyl-ethyl)-amine as the TFA salt. ¹H NMR (d₆-DMSO, 400 MHz): 1.54 (d, 3H, J=7.0 Hz), 2.56 (s, 3H), 5.09 (bs, 1H), 6.68 (d, 1H, J=5.2 Hz), 7.31 (m, 1H), 7.44 (m, 6H), 7.65 (m, 1H), 7.77 (d, 1H, J=7.3 Hz), 7.85 (d, 1H, J=7.3 Hz), 7.96 (dd, 1H, J=8.3, 7.3 Hz), 8.23 (m, 1H), 8.30 (d, 1H, J=5.0 Hz); MS (ESP+) 407.2 (M+1).

### EXAMPLE 40

### 8-Methyl-2-(6-methyl-pyridin-2-yl)-3-(2-methylsulfanyl-pyrimidin-4-yl)-imidazo[1,2-a]pyridine

Hydrogen bromide/acetic acid (30 wt %, 7.70 mmol) and 1.0 M bromine/acetic acid (2.0 mmol) was added to a solution of 1-(6-methyl-pyridin-2-yl)-2-(2-methylsulfanyl-pyrimidin-4-yl)-ethanone (1.54 mmol; see Example 1(b) for its preparation) and catalytic BHT in glacial acetic acid (10 mL) at RT with formation of a precipitate. After 1 h the reaction was diluted with ether (40 mL), stirred for 30 minutes, the precipitate was then filtered and washed with Et₂O (Extreme caution was taken to make sure the precipitate did not dry completely). The precipitate was slurried in CH₃CN (10 mL), added to a slurry of Hunig's base (4.58 mmol) and 3-methyl-2-aminopyridine (1.85 mmol) at RT and then warmed overnight at 55° C. A precipitate formed upon cooling to RT. The slurry was diluted with water (10 mL), filtered, washed with cold CH₃CN to give 211 mg of a tan solid identified as 8-methyl-2-(6-methyl-pyridin-2-yl)-3-(2-methylsulfanyl-pyrimidin-4-yl)-imidazo[1,2-a]pyridine.

### EXAMPLE 41

### 3-(2-methanesulfonyl-pyrimidin-4-yl)-8-methyl-2-(6-methyl-pyridin-2-yl)-imidazo[1,2-a]pyridine

4.0N Sulfuric acid (0.06 mmol), catalytic sodium tungstate dihydrate, and 30 wt % hydrogen peroxide (1.96 mmol) was added to a slurry of 8-methyl-2-(6-methyl-pyridin-2-yl)-3-(2-methylsulfanyl-pyrimidin-4-yl)-imidazo[1,2-a]pyridine (0.608 mmol; see Ex. 40 for its preparation) in methanol (4 mL) at 50° C. After 3.5 h the reaction was diluted with water (10 mL) and warmed at 50° C. for a further 0.5 h. The reaction was then cooled to RT, quenched with saturated sodium thiosulfate, the pH adjusted to 6 with 1M NaOH and filtered to give 0.200 g of a tan solid identified as 3-(2-methanesulfonyl-pyrimidin-4-yl)-8-methyl-2-(6-methyl-pyridin-2-yl)-imidazo[1,2-a]pyridine. ¹H NMR (d₆-DMSO, 400 MHz): 2.44 (s, 3H), 2.66 (s, 3H), 3.53 (s, 3H), 7.15 (dd, 1H, J=7.0, 7.0 Hz), 7.36 (d, 1H, J=7.7 Hz), 7.44 (d, 1H, J=6.6 Hz), 7.97 (m, 31), 8.05 (d, 1H, J=5.3 Hz), 8.99 (d, 1H, J=5.1 Hz), 9.24 (d, 1H, J=7.0 Hz); MS (ESP+) 380.2 (M+1).

### EXAMPLE 42

### 4-[8-methyl-2-(6-methyl-pyridin-2-yl)-imidazo[1,2-a]pyridin-3-yl]-pyrimidin-2-ylamine

In a sealed tube, a slurry of 3-(2-methanesulfonyl-pyrimidin-4-yl)-8-methyl-2-(6-methyl-pyridin-2-yl)-imidazo[1,2-a]pyridine (0.263 mmol; prepared in accordance with Ex. 2) and NH₄OAc (6.60 mmol) in 2:1 dioxane/water (3 mL) was warmed at 100° C. for 7 days. The reaction was cooled to RT, diluted with EtOAc (25 ml), washed with H₂O, brine, dried (Na₂SO₄), concentrated in vacuo and purified via preparatory HPLC (CH₃CN/H₂O gradient with 0.1% TFA) to give 30 mg of a solid identified as the TFA salt of 4-[8-methyl-2-(6-methyl-pyridin-2-yl)-imidazo[1,2-a]pyridin-3-yl]-pyrimidin-2-ylamine. ¹H NMR (d₆-DMSO, 400 MHz): 2.65 (s, 3H), 6.75 (d, 1H, J=5.8 Hz), 7.17 (dd, 1H, J=7.1, 7.1 Hz), 7.46 (d, 1H, J=8.1 Hz) 7.50 (d, 1H, J=6.5 Hz), 7.83 (d, 1H, J=7.7 Hz), 7.97 (dd, 1H, J=7.6, 7.6 Hz), 8.20 (d, 1H, J=6.0 Hz), 9.57 (d, 1H, J=7.3 Hz); MS (ESP+) 317.2 (M+1).

### EXAMPLE 43

### 4-[7-Methyl-2-(6-methyl-pyridin-2-yl)-imidazo[1,2-a]pyridin-3-yl]-pyrimidin-2-ylamine

Hydrogen bromide/acetic acid (30 wt %, 7.70 mmol) and 1.0 M bromine/acetic acid (2.0 mmol) was added to a solution of 1-(6-methyl-pyridin-2-yl)-2-(2-methylsulfanyl-pyrimidin-4-yl)-ethanone (1.54 mmol; see Example 1(b) for its preparation) and catalytic BHT in glacial acetic acid (10 mL) at RT with formation of a precipitate. After 1 h the reaction was diluted with ether (40 mL), stirred for 30 minutes, the precipitate was then filtered and washed with Et₂O (extreme caution was taken to make sure the precipitate did not dry completely). The precipitate was slurried in CH₃CN, added to a mixture of Hunig's base and 4-methyl-2-aminopyridine (1.86 mmol) at RT and then warmed overnight at 55° C. A precipitate formed upon cooling to RT. The slurry was diluted with water (10 mL), filtered, washed with cold CH₃CN to give 211 mg of a solid identified as 7-methyl-2-(6-methyl-pyridin-2-yl)-3-(2-methylsulfanyl-pyrimidin-4-yl)-imidazo[1,2-a]pyridine. ¹H NMR (CDCl₃, 300 MHz): 2.49 (s, 3H), 2.56 (s, 3H), 2.64 (s, 3H), 6.84 (m, 1H), 7.20 (m, 2H), 7.58 (m, 1H), 7.73 (m, 2H), 8.35 (d, 1H J=5.4 Hz), 9.41 (d, 1H, J=6.9 Hz); MS (ESP+) 373.3 (M+1).

### EXAMPLE 44

### 3-(2-methanesulfonyl-pyrimidin-4-yl)-7-methyl-2-(6-methyl-pyridin-2-yl)-imidazo[1,2-a]pyridine

4.0N Sulfuric acid (0.02 mmol), catalytic sodium tungstate dihydrate, and 30 wt % hydrogen peroxide (X mmol) was added to a slurry 7-methyl-2-(6-methyl-pyridin-2-yl)-3-(2-methylsulfanyl-pyrimidin-4-yl)-imidazo[1,2-a]pyridine (0.274 mmol; prepared in accordance with Ex. 1) in methanol (3 mL) at 55° C. After 3.5 h the reaction was diluted with water (10 mL) and warmed at 55° C. for a further 0.5 h. The reaction was then cooled to RT, quenched with saturated sodium thiosulfate, the pH adjusted to 6 with 1M NaOH and filtered to give 0.070 g of a yellow solid identified 3-(2-methanesulfonyl-pyrimidin-4-yl)-7-methyl-2-(6-methyl-pyridin-2-yl)-imidazo[1,2-a]pyridine. ¹H NMR (d₆-DMSO, 400 MHz): 2.42 (s, 3H), 2.46 (s, 3H), 3.49 (s, 3H), 7.11 (dd, 1H, J=1.6, 7.2 Hz), 7.36 (d, 1H, J=7.0 Hz), 7.67 (bs, 1H), 7.92 (m, 2H), 8.08 (d, 1H J=5.5 Hz), 8.98 (d, 1H, J=5.5 Hz); MS (ESP+) 380.2 (M+1).

### EXAMPLE 45

### 4-[7-methyl-2-(6-methyl-pyridin-2-yl)-imidazo[1,2-a]pyridin-3-yl]-pyrimidin-2-ylamine

In a sealed tube, a slurry of 3-(2-methanesulfonyl-pyrimidin-4-yl)-7-methyl-2-(6-methyl-pyridin-2-yl)-imidazo[1,2-a]pyridine (0.184 mmol; see Example 44 for its preparation) and NH₄OAc (4.60 mmol) in 2:1 dioxane/water (3 mL) was warmed to 100° C. for 7 days. The reaction was cooled to RT, diluted with EtOAc (25 ml), washed with H₂O, brine, dried (Na₂SO₄), concentrated in vacuo and purified via preparatory HPLC (CH₃CN/H₂O gradient with 0.1% TFA) to yield 22 mg of a solid identified as the TFA salt of 4-[7-methyl-2-(6-methyl-pyridin-2-yl)-imidazo[1,2-a]pyridin-3-yl]-pyrimidin-2-ylamine. ¹H NMR (d₆-DMSO, 400 MHz): 2.53 (s, 3H), 2.57 (s, 3H), 6.80 (d, 1H, J=6.0 Hz), 7.19 (d, 1H, J=7.9 Hz), 7.47 (d, 1H, J=7.6 Hz), 7.71 (bs, 1H), 7.78 (d, 1H, J=7.8 Hz), 7.96 (dd, 1H, J=7.6, 7.6 Hz), 8.23 (d, 1H, J=6.2 Hz), 9.56 (d, 1H, J=6.4 Hz); MS (ESP+) 317.3 (M+1).

### EXAMPLE 46

### 6-Chloro-2-(6-methyl-pyridin-2-yl)-3-(2-methylsulfanyl-pyrimidin-4-yl)-imidazo[1,2-a]pyridine

Hydrogen bromide/acetic acid (30 wt %, 7.70 mmol) and 1.0 M bromine/acetic acid (2.0 mmol) was added to a solution of 1-(6-methyl-pyridin-2-yl)-2-(2-methylsulfanyl-pyrimidin-4-yl)-ethanone (1.54 mmol; see Example 1(b) for its preparation) and catalytic BHT in glacial acetic acid (10 mL) at RT with formation of a precipitate. After 1 h the reaction was diluted with ether (40 mL), stirred for 30 minutes, the precipitate was then filtered and washed with Et₂O (Extreme caution was taken to make sure the precipitate did not dry completely). The precipitate was slurried in CH₃CN (10 mL). 4-Chloro-2-aminopyridine (1.85 mmol) and Hunig's base (4.58 mmol) was added to this slurry and the mixture was allowed to stir overnight at 55 C under a nitrogen atmosphere. A precipitate formed upon cooling to RT. The slurry was diluted with water (10 mL), filtered and washed with cold CH₃CN to give 124 mg of a impure tan solid identified as 6-chloro-2-(6-methyl-pyridin-2-yl)-3-(2-methylsulfanyl-pyrimidin-4-yl)-imidazo[1,2-a]pyridine. MS (ESP+) 368.0 (M+1).

### EXAMPLE 47

### 6-Chloro-3-(2-methanesulfonyl-pyrimidin-4-yl)-2-(6-methyl-pyridin-2-yl)-imidazo[1,2-a]pyridine

4.0N Sulfuric acid (0.02 mmol), catalytic sodium tungstate dihydrate, and 30 wt % hydrogen peroxide (0.98 mmol) was added to a slurry 6-chloro-2-(6-methyl-pyridin-2-yl)-3-(2-methylsulfanyl-pyrimidin-4-yl)-imidazo[1,2-a]pyridine (0.274 mmol; see Ex. 46 for its preparation) in methanol (2 mL) at 55° C. After 3.5 h the reaction was diluted with water (10 mL) and warmed at 55° C. for a further 0.5 h. The reaction was then cooled to RT, quenched with saturated sodium thiosulfate, the pH adjusted to 6 with 1M NaOH and filtered to give 0.050 g of a yellow solid identified as impure 6-chloro-3-(2-methanesulfonyl-pyrimidin-4-yl)-2-(6-methyl-pyridin-2-yl)-imidazo[1,2-a]pyridine. MS (ESP+) 400.0 (M+1).

### EXAMPLE 48

### 4-[6-Chloro-2-(6-methyl-pyridin-2-yl)-imidazo[1,2-a]pyridin-3-yl]-pyrimidin-2-ylamine

In a sealed tube, a slurry of 6-chloro-3-(2-methanesulfonyl-pyrimidin-4-yl)-2-(6-methyl-pyridin-2-yl)-imidazo[1,2-a]pyridine (0.125 mmol; see Example 47 for its preparation) and NH₄OAc (6.60 mmol) in 1:1 dioxane/water (2 mL) was warmed at 100° C. for 5 days. The reaction cooled to RT, diluted with EtOAc (25 ml), washed with H₂O, brine, dried (Na₂SO₄), concentrated in vacuo and purified via preparatory HPLC (CH₃CN/H₂O gradient with 0.1% TFA) to yield 4 mg of a solid identified as the TFA salt of 4-[6-chloro-2-(6-methyl-pyridin-2-yl)-imidazo[1,2-a]pyridin-3-yl]-pyrimidin-2-ylamine. ¹H NM (d₆-DMSO, 400 MHz): 2.50 (s, 3H), 6.84 (d, 1H, J=6.0 Hz), 7.45 (d, 1H, J=8.5 Hz), 7.72 (d, 1H, J=10.1 Hz), 7.86 (d, 1H, J=6.9 Hz), 7.96 (m, 2H), 8.25 (d, 1H, J=6.0 Hz), 9.72 (s, 1H); MS (ESP+) 337.1 (M+1).

### EXAMPLE 49

### [2-(6-Methyl-pyridin-2-yl)-3-(2-methylsulfanyl-pyrimidin-4-yl)-imidazo[1,2-a]pyridin-6-yl]-methanol

Hydrogen bromide/acetic acid (30 wt %, 7.70 mmol) and 1.0 M bromine/acetic acid (2.0 mmol) was added to a solution of 1-(6-methyl-pyridin-2-yl)-2-(2-methylsulfanyl-pyrimidin-4-yl)-ethanone (1.54 mmol; see Example 1(b) for its preparation) and catalytic BHT in glacial acetic acid (10 mL) at −15° C. with formation of a precipitate. After 0.25 h the reaction was diluted with ether (40 mL), stirred for 5 minutes, the precipitate was then filtered and washed with Et₂O under a nitrogen flow (Extreme caution was taken to make sure the precipitate did not dry completely). The precipitate was added to a flask containing (6-amino-pyridin-3-yl)-methanol (2.06 mmol) under a nitrogen atmosphere, slurried in toluene (8 mL) and warmed to 100° C. Diisopropylethylamine (11.4 mmol) was added slowly and the reaction was allowed to stir at 100° C. for about 4 hours. A precipitate formed upon cooling to RT. The slurry was cooled to 0° C., filtered, washed with CH₃CN and air dried to give 345 mg of a tan solid identified as [2-(6-methyl-pyridin-2-yl)-3-(2-methylsulfanyl-pyrimidin-4-yl)-imidazo[1,2-a]pyridin-6-yl]-methanol. ¹H NR (CDCl₃, 400 MHz): 2.62 (s, 3H), 2.63 (s, 3H), 4.74 (s, 2H), 7.01 (d, 1H, J=5.5 Hz), 7.24 (d, 1H, J=7.7 Hz), 7.29 (d, 1H, J=7.29 Hz), 7.59 (m, 2H), 7.71 (t, 1H, J=7.5 Hz), 8.29 (d, 1H, J=5.7 Hz), 9.40 (bs, 1H); MS (ESP+) 364.2 (M+1).

### EXAMPLE 50

### [3-(2-Methanesulfonyl-pyrimidin-4-yl)-2-(6-methyl-pyridin-2-yl)-imidazo[1,2-a]pyridin-6-yl]-methanol

4.0 N Sulfuric acid (0.12 mmol), catalytic sodium tungstate dihydrate, and 30 wt % hydrogen peroxide (9.8 mmol) was added to a slurry of [2-(6-methyl-pyridin-2-yl)-3-(2-methylsulfanyl-pyrimidin-4-yl)-imidazo[1,2-a]pyridin-6-yl]-methanol (0.95 mmol; see Example 49 for its preparation) in methanol (50 mL) at 50° C. After 3.5 h the reaction was diluted with water (9.5 mL) and warmed at 55° C. for a further 0.5 h. The reaction was then cooled to RT, quenched with saturated sodium thiosulfate, the pH adjusted to 6 with 1M NaOH and filtered to give 0.208 g of a tan solid identified as [3-(2-methanesulfonyl-pyrimidin-4-yl)-2-(6-methyl-pyridin-2-yl)-imidazo[1,2-a]pyridin-6-yl]-methanol. ¹H NMR (d₆-DMSO, 400 MHz): 2.40 (s, 3H), 3.52 (s, 3H), 4.57 (d, 2H, J=5.4 Hz), 7.32 (d, 1H, J=7.3 Hz), 7.54 (dd, 1H, J=1.3, 9.0 Hz), 7.87 (m, 3H), 8.05 (d, 1H, J=5.2 Hz), 8.98 (d, 1H, J=5.4 Hz), 9.31 (bs, 1H); MS (ESP+) 400.2 (M+1).

### EXAMPLE 51

### [3-(2-Amino-pyrimidin-4-yl)-2-(6-methyl-pyridin-2-yl)-imidazo[1,2-a]pyridin-6-yl]-methanol

In a sealed tube, a solution of 3-(2-methanesulfonyl-pyrimidin-4-yl)-2-(6-methyl-pyridin-2-yl)-imidazo[1,2-a]pyridin-6-yl]-methanol (0.526 mmol; see Example 50 for its preparation) and NH₄OAc (10.5 mmol) in 1:1 dioxane/water (7 mL) was warmed to 100° C. for 3 days. The mixture was cooled to RT, concentrated in vacuo and purified via preparatory HPLC (5→50% CH₃CN/H₂O with 0.1% TFA) to yield 49.3 mg of a yellow solid identified as the TFA salt of [3-(2-amino-pyrimidin-4-yl)-2-(6-methyl-pyridin-2-yl)-imidazo[1,2-a]pyridin-6-yl]-methanol. ¹H NMR (d₆-DMSO, 400 MHz): 2.530 (s, 3), 4.64 (s, 2H), 6.80 (d, 1H, J=6.0 Hz), 7.44 (d, 1H, J=8.6 Hz), 7.65 (bs, 1H), 7.67 (m, 1H), 7.79 (d, 1H, J=8.0 Hz), 7.85 (d, 1H, J=9.3 Hz), 7.94 (dd, 1H, J=7.3, 7.3 Hz), 8.24 (d, 1H, J=6.0 Hz), 9.42 (bs, 1H). MS (ESP+) 333.4 (M+1).

### EXAMPLE 52

### 2-(6-Methyl-pyridin-2-yl)-3-(2-methylsulfanyl-pyrimidin-4-yl)-imidazo[1,2-a]pyridine-6-carboxylic acid methyl ester

Hydrogen bromide/acetic acid (30 wt %, 7.53 mmol) and 0.98 M bromine/acetic acid (2.48 mmol) was added to a solution of 1-(6-methyl-pyridin-2-yl)-2-(2-methylsulfanyl-pyrimidin-4-yl)-ethanone (1.911 mmol; see Example 1(b) for its preparation) and catalytic BHT in glacial acetic acid (10 mL) at RT with formation of a precipitate. After 0.25 h the reaction was diluted to 100 mL with ether, filtered, washed with ether, air dried briefly under a nitrogen stream and added to a flask containing 6-amino-nicotinic acid methyl ester (2.036 mmol) under a nitrogen atmosphere. Anhydrous acetonitrile (5 mL) and diisopropylethylamine (7.65 mmol) were added and the resulting solution was warmed to 80° C. After 5.5 h the reaction was allowed to cool to RT and precipitate was formed. The slurry was filtered, washed with acetonitrile and air dried to give 0.4816 g of a tan solid identified as impure 2-(6-methyl-pyridin-2-yl)-3-(2-methylsulfanyl-pyrimidin-4-yl)-imidazo[1,2-a]pyridine-6-carboxylic acid methyl ester. ¹H NMR (CDCl₃, 400 MHz) 2.35 (s, 3H), 2.69 (s, 3H), 3.98 (s, 3H), 7.21 (d, J=5.26 Hz, 1H), 7.22 (d, J=6.81 Hz, 1), 7.73 (d, J=7.31 Hz, 1H), 7.82 (J=9.67 Hz, 1H), 7.94 (dd, J=9.36, 1.54 Hz, 1H), 8.38 (d, J=5.33 Hz, 1H), 10.34 (s, 1H); MS (ESP+) 392.11 (M+1).

### EXAMPLE 53

### 3-(2-Methanesulfonyl-pyrimidin-4-yl)-2-(6-methyl-pyridin-2-yl)-imidazo[1,2-a]pyridine-6-carboxylic acid methyl ester

4.0 N Sulfuric acid (0.02 mmol), catalytic sodium tungstate dihydrate, and 30 wt % hydrogen peroxide (0.88 mmol) was added to a slurry of 2-(6-methyl-pyridin-2-yl)-3-(2-methylsulfanyl-pyrimidin-4-yl)-imidazo[1,2-a]pyridine-6-carboxylic acid methyl ester (0.2664 mmol; see Example 52 for its preparation) in 1:1 methanolmethylene chloride (4 mL) at 55° C. After 6.6 h, the reaction was diluted with water (2 mL) and warmed at 55° C. for 0.75 h. The reaction was then cooled to RT and neutralized to I₂/starch paper with saturated sodium thiosulfate. The reaction was then diluted with methylene chloride (20 mL) and the organic phase was washed with 10% sodium bicarbonate (5 mL) and brine (5 mL), dried (MgSO₄) and concentrated in vacuo to give 0.1026 g of a yellow identified as 3-(2-methanesulfonyl-pyrimidin-4-yl)-2-(6-methyl-pyridin-2-yl)-imidazo[1,2-a]pyridine-6-carboxylic acid methyl ester. ¹H NMR (CDCl₃, 300 MHz) 2.54 (s, 3H), 3.45 (s, 3H), 3.99 (s, 3H), 7.27 (d, J=6.60 Hz, 1H), 7.78 (d, J=9.30 Hz, 1H), 7.78 (dd, J=7.80, 7.80 Hz, 1H), 7.90 (J=8.10 Hz, 1H), 8.01 (dd, J 9.30, 1.50 Hz, 1H), 8.03 (d, J=5.40 Hz, 1H), 8.74 (d, J=5.40 Hz, 1H), 10.58 (s, 1H); MS (ESP+) 424.15 (M+1).

### EXAMPLE 54

### 3-(2-Amino-pyrimidin-4-yl)-2-(6-methyl-pyridin-2-yl)-imidazo[1,2-a]pyridine-6-carboxylic acid methyl ester

A solution of 3-(2-methanesulfonyl-pyrimidin-4-yl)-2-(6-methyl-pyridin-2-yl)-imidazo[1,2-a]pyridine-6-carboxylic acid methyl ester (0.1717 mmol; see Example 53 for its preparation) and ammonium acetate (3.526 mmol) in 2:1 dioxane/water (3 mL) was warmed to 100° C. in a sealed tube. After 23.5 h, the reaction was allowed to cool to RT, filtered, the solid washed with acetonitrile and air dried to give 0.0340 g of a tan solid. This solid was purified via reverse phase HPLC (acetonitrile/water gradient with 0.1% TFA) to give 0.0295 g of a yellow solid identified as the TFA salt of 3-(2-amino-pyrimidin-4-yl)-2-(6-methyl-pyridin-2-yl)-imidazo[1,2-a]pyridine-6-carboxylic acid methyl ester. ¹H NMR (DMSO-d6, 400 MHz) 2.41 (s, 3H), 3.87 (s, 3H), 6.78 (d, J=5.69 Hz, 1H), 7.33 (d, J=7.44 Hz, 1H), 7.4 (br s, 2H), 7.80-7.86 (m, 4H), 8.22 (d, J=5.65, 1H), 9.77 (s, 1H); MS (ESP+) 361.4 (+1).

### EXAMPLE 55

### 3-(2-Amino-pyrimidin-4-yl)-2-(6-methyl-pyridin-2-yl)-imidazo[1,2-a]pyridine-6-carboxylic acid

In a sealed tube, a solution of 3-(2-methylsulfonyl-pyrimidin-4-yl)-2-(6-methyl-pyridin-2-yl)-imidazo[1,2-a]pyridine-6-carboxylic acid methyl ester (2.30 mmol; see Example 53 for its preparation) and NH₄OAc (45.8 mmol) in 1:1 dioxane/water (30 mL) was warmed to 100° C. for 3 days. The mixture was cooled to RT, resulting in a precipitate that was filtered and washed with H₂0 to give 430 mg of the carboxylic acid and ester mixture. The mixture was dissolved in 2:1 THF/H₂0 (15 mL), LiOH.H₂0 (5.95 mmol) was added and the reaction stirred at RT for 0.5 h. The reaction was neutralized with 2M HCl, cooled to 0° C., filtered and washed with H₂0 to give 370 mg of a solid identified as 3-(2-amino-pyrimidin-4-yl)-2-(6-methyl-pyridin-2-yl)-imidazo[1,2-a]pyridine-6-carboxylic acid. ¹H NMR (d₆-DMSO, 400 MHz): 2.39 (s, 3H), 6.75 (d, 1H J=5.3 Hz), 6.79 (bs, 21), 7.30 (dd, 1H, J=4.0, 4.3 Hz), 7.83 (m, 5H), 8.29 (d, 1H, J=5.3 Hz), 9.65 (bs, 1H); MS (ESP+) 347.7 (M+1).

### EXAMPLE 56

### 3-(2-Amino-pyrimidin-4-yl)-2-(6-methyl-pyridin-2-yl)-imidazo[1,2-a]pyridine-6-carboxylic acid methoxy-amide

In a vial, 3-(2-amino-pyrimidin-4-yl)-2-(6-methyl-pyridin-2-yl)-imidazo[1,2-a]pyridine-6-carboxylic acid (0.144 mmol; see Example 55 for its preparation) was added with DMF (1 mL). The reaction was stirred until all material had dissolved. HATU (0.201 mmol) was then added making sure all material had dissolved. This was followed by the addition of DIEA (1.15 mmol). This mixture was allowed to stir for 10 min. Methoxyamine hydrochloride (0.173 mmol) was then added, the reaction was stirred for 2 h, concentrated in vacuo and purified via preparative HPLC (5→50% CH₃CN:H₂0 with 0.1% TFA) to give 28.9 mg of a solid identified as the TFA salt of 3-(2-amino-pyrimidin-4-yl)-2-(6-methyl-pyridin-2-yl)-imidazo[1,2-a]pyridine-6-carboxylic acid methoxy-amide. ¹H NMR (d₆-DMSO, 400 MHz): 2.52 (s, 3H), 3.80 (s, 3H), 6.92 (d, 1H, J=5.7 Hz), 7.45 (d, 1H, J=7.6 Hz), 7.61 (bs, 1H), 7.90 (m, 5H), 8.32 (d, 1H, J=5.4 Hz), 9.69 (s, 1H); MS 376.5 (ESP+) (M+1).

### EXAMPLE 57

### 3-(2-Amino-pyrimidin-4-yl)-2-(6-methyl-pyridin-2-yl)-imidazo[1,2-a]pyridine-6-carboxylic acid ethylamide

In a vial, 3-(2-amino-pyrimidin-4-yl)-2-(6-methyl-pyridin-2-yl)-imidazo[1,2-a]pyridine-6-carboxylic acid (0.144 mmol; see Example 55 for its preparation) was added with DMF (1 mL). The reaction was stirred until all material had dissolved. HATU (0.201 mmol) was then added making sure all material had dissolved. This was followed by the addition of DIEA (0.720 mmol). This mixture was allowed to stir for 10 min. 2.0M Ethyl amine/THF (0.432 mmol) was then added, the reaction was stirred for 2 h, concentrated in vacuo and purified via preparative HPLC (5→40% CH₃CN:H₂0 with 0.1% TFA) to give 41.0 mg of a solid identified as the TFA salt of 3-(2-amino-pyrimidin-4-yl)-2-(6-methyl-pyridin-2-yl)-imidazo[1,2-a]pyridine-6-carboxylic acid ethylamide. ¹H NMR (d₆-DMSO, 400 M[z): 1.17 (t, 3H, 7.3 Hz), 2.53 (s, 3H), 3.37 (m, 2H), 6.92 (d, 1H, J=5.8 Hz), 7.44 (d, 1H, J=7.7 Hz), 7.70 (bs, 1H), 7.93 (m, 4H), 8.33 (d, 1H, J=8.3 Hz), 8.73 (dd, 1H, J=5.4, 5.4 Hz), 9.71 (m, 1H); MS (ESP+) 374.3 (M+1).

### EXAMPLE 58

### 3-(2-Amino-pyrimidin-4-yl)-2-(6-methyl-pyridin-2-yl)-imidazo[1,2-a]pyridine-6-carboxylic acid (2-dimethylamino-ethyl)-amide

In a vial, 3-(2-amino-pyrimidin-4-yl)-2-(6-methyl-pyridin-2-yl)-imidazo[1,2-a]pyridine-6-carboxylic acid (0.144 mmol; see Example 55 for its preparation) was added with DMF (1 mL). The reaction was stirred until all material had dissolved. HATU (0.201 mmol) was then added making sure all material had dissolved. This was followed by the addition of DIEA (0.720 mmol). This mixture was allowed to stir for 10 min. N,N-Dimethylethyldiamine (0.173 mmol) was then added, the reaction was stirred for 2 h, concentrated in vacuo and purified via preparative HPLC (5→40% CH₃CN:H₂0 with 0.1% TFA) to give 29.9 mg of a solid identified as the TFA salt of 3-(2-amino-pyrimidin-4-yl)-2-(6-methyl-pyridin-2-yl)-imidazo[1,2-a]pyridine-6-carboxylic acid (2-dimethylamino-ethyl)-amide. ¹H NMR (d₆-DMSO, 400 MHz): 2.50 (s, 3H), 2.91 (d, 6H, J=4.6 Hz), 3.34 (q, 2H, J=5.5 Hz), 3.69 (q, 2H, J=5.7 Hz), 6.90 (d, 1H, J=5.8 Hz), 7.41 (d, 1H, J=7.2 Hz), 7.51 (bs, 1H), 7.92 (m, 4H), 8.33 (d, 1H, J=5.9 Hz), 8.96 (dd, 1H, J=5.7, 5.7 Hz), 9.60 (bs, 1H), 9.78 (m, 1H); MS (ESP+) 417.5(M+1).

### EXAMPLE 59

### 3-(2-Amino-pyrimidin-4-yl)-2-(6-methyl-pyridin-2-yl)-imidazo[1,2-a]pyridine-6-carboxylic acid (2-methoxy-ethyl)-amide

In a vial, 3-(2-amino-pyrimidin-4-yl)-2-(6-methyl-pyridin-2-yl)-imidazo[1,2-a]pyridine-6-carboxylic acid (0.144 mmol; see Example 55 for its preparation) was added with DMF (1 mL). The reaction was stirred until all material had dissolved. HATU (0.201 mmol) was then added making sure all material had dissolved. This was followed by the addition of DIEA (0.720 mmol). This mixture was allowed to stir for 10 min. Methyoxyethylamine (0.288 mmol) was then added, the reaction was stirred for 2 h, concentrated in vacuo and purified via preparative HPLC (5→40% CH₃CN:H₂0 with 0.1% TFA) to give 36.1 mg of a solid identified as the TFA salt of 3-(2-amino-pyrimidin-4-yl)-2-(6-methyl-pyridin-2-yl)-imidazo[1,2-a]pyridine-6-carboxylic acid (2-methoxy-ethyl)-amide. ¹H NMR (d₆-DMSO, 400 MHz): 2.51 (s, 3H), 3.32 (s, 3H), 3.52 (m, 4H), 6.89 (d, 1H, J=5.6 Hz), 7.42 (d, 1H, J=8.1 Hz), 7.45 (bs, 1H), 7.92 (m, 4H), 8.33 (d, 1H, J=5.6 Hz), 8.81 (m, 1H), 9.70 (s, 1H): MS (ESP+) 404.7(M+1).

### EXAMPLE 60

### 3-(2-Amino-pyrimidin-4-yl)-2-(6-methyl-pyridin-2-yl)-imidazo[1,2-a]pyridine-6-carboxylic acid ([1,4]dioxan-2-ylmethyl)-amide

In a vial, 3-(2-amino-pyrimidin-4-yl)-2-(6-methyl-pyridin-2-yl)-imidazo[1,2-a]pyridine-6-carboxylic acid (0.144 mmol; see Example 55 for its preparation) was added with DMF (1 mL). The reaction was stirred until all material had dissolved. HATU (0.201 mmol) was then added making sure all material had dissolved. This was followed by the addition of DIEA (0.720 mmol). This mixture was allowed to stir for 10 min. [1,4]Dioxan-2-yl-methylamine (0.202 mmol) was then added, the reaction was stirred for 2 h, concentrated in vacuo and purified via preparative HPLC (5→40% CH₃CN:H₂0 with 0.1% TFA) to give 30.0 mg of a solid identified as the TFA salt of 3-(2-amino-pyrimidin-4-yl)-2-(6-methyl-pyridin-2-yl)-imidazo[1,2-a]pyridine-6-carboxylic acid ([1,4]dioxan-2-ylmethyl)-amide. ¹H NMR (d₆-DMSO, 400 MHz): 2.52 (s, 3H), 3.30 (m, 1H), 3.37 (m, 2H), 3.51 (m, 1H), 3.62 (m, 1H), 3.68 (m, 1H), 3.73 (m, 1H), 3.80 (m, 2H), 6.92 (d, 1H, J=5.6 Hz), 7.43 (d, 1H, J=7.7 Hz), 7.59 (bs, 1H), 7.93 (m, 4H), 8.32 (d, 1H J=5.6 Hz), 8.82 (dd, 1H, J=5.6, 5.6 Hz), 9.72 (s, 1H); MS (ESP+) 446.3 (M+1).

### EXAMPLE 61

### 3-{[3-(2-Amino-pyrimidin-4-yl)-2-(6-methyl-pyridin-2-yl)-imidazo[1,2-a]pyridine-6-carbonyl]-amino}-propionic acid methyl ester

In a vial, 3-(2-amino-pyrimidin-4-yl)-2-(6-methyl-pyridin-2-yl)-imidazo[1,2-a]pyridine-6-carboxylic acid (0.144 mmol; see Example 55 for its preparation) was added with DMF (1 mL). The reaction was stirred until all material had dissolved. HATU (0.201 mmol) was then added making sure all material had dissolved. This was followed by the addition of DIEA (0.720 mmol). This mixture was allowed to stir for 10 min. 3-Amino-propionic acid methyl ester (0.202 mmol) was then added, the reaction was stirred for 2 h, concentrated in vacuo and purified via preparative HPLC (5→40% CH₃CN:H₂O with 0.1% TFA) to give 27.3 mg of a solid identified as the TFA salt of 3-{[3-(2-amino-pyrimidin-4-yl)-2-(6-methyl-pyridin-2-yl)-imidazo[1,2-a]pyridine-6-carbonyl]-amino}-propionic acid methyl ester. ¹H NMR (d₆-DMSO, 400 MHz): 2.51 (s, 3H), 2.68 (t, 2H, J=7.4 Hz), 3.57 (q, 2H, J=6.8 Hz), 3.66 (s, 3H), 6.88 (d, 1H, J=5.4 Hz), 7.42 (d, 1H, J=7.4 Hz), 7.43 (bs, 1H), 7.91 (m, 4H), 8.31 (d, 1H, J=6.0 Hz), 8.84 (dd, 1H, J=5.4, 5.4 Hz), 9.71 (s, 1H; MS (ESP+) 432.2 (M+1).

### EXAMPLE 62

### 3-(2-Amino-pyrimidin-4-yl)-2-(6-methyl-pyridin-2-yl)-imidazo[1,2-a]pyridine-6-carboxylic acid ([1,4]dioxan-2-ylmethyl)-amide

In a vial, 3-(2-amino-pyrimidin-4-yl)-2-(6-methyl-pyridin-2-yl)-imidazo[1,2-a]pyridine-6-carboxylic acid (0.144 mmol; see Example 55 for its preparation) was added with DMF (1 mL). The reaction was stirred until all material had dissolved. HATU (0.201 mmol) was then added making sure all material had dissolved. This was followed by the addition of DIEA (0.720 mmol). This mixture was allowed to stir for 10 min. (unsym)-N,N-Dimethylhydrazine (0.202 mmol) was then added, the reaction was stirred for 2 h, concentrated in vacuo and purified via preparative HPLC (5→40% CH₃CN:H₂0 with 0.1% TFA) to give 9.8 mg of a solid identified as the TFA salt of 3-(2-amino-pyrimidin-4-yl)-2-(6-methyl-pyridin-2-yl)-imidazo[1,2-a]pyridine-6-carboxylic acid ([1,4]dioxan-2-ylmethyl)-amide. ¹H NMR (d₆-DMSO, 400 MHz): 2.52 (s, 3H), 2.74 (s, 6H), 6.92 (d, 1H, J=5.6 Hz), 7.43 (d, 1H, J=7.4 Hz), 7.57 (bs, 1H), 7.92 (m, 4H), 8.32 (d, 1H, J=5.6 Hz), 9.70 (s, 1H); MS (ESP+) 389.2 (M+1).

### EXAMPLE 63

### N-[3-(2-Amino-pyrimidin-4-yl)-2-(6-methyl-pyridin-2-yl)-imidazo[1,2-a]pyridine-6-carbonyl]-methanesulfonamide

In a vial, 3-(2-amino-pyrimidin-4-yl)-2-(6-methyl-pyridin-2-yl)-imidazo[1,2-a]pyridine-6-carboxylic acid (60 mg, 0.173 mmol; see Example 55 for its preparation) was added with DMF (1 mL). The reaction was stirred until all material had dissolved. HATU (92 mg, 0.242 mmol) was then added making sure all material had dissolved. This was followed by the addition of DIEA (150 uL, 0.865 mmol). This mixture was allowed to stir for 10 min. Methylsulfonamine (20 mg, 0.207 mmol) was then added and the reaction was stirred for 2 h and checked by HPLC. The reaction was purified by preparative HPLC (5→40% CH3 CN:H20 with 0.1% TFA). (Impurity in NMR) ¹H NMR (d₆-DMSO, 400 MHz): 2.47 (s, 3H), 3.45 (s, 3H), 6.88 (d, 1H, J=5.7 Hz), 7.17 (bs, 1H), 7.36 (dd, 1H, J=2.8, 5.7 Hz), 7.88 (m, 4H), 8.34 (d, 1H, J=5.0 Hz), 9.81 (s, 1H). MS (ESP+) 424.3 (M+1).

### EXAMPLE 64

### 3-(2-Amino-pyrimidin-4-yl)-2-(6-methyl-pyridin-2-yl)-imidazo[1,2-a]pyridine-6-carboxylic acid (2-thiophen-2-yl-ethyl)-amide

In a vial, 3-(2-amino-pyrimidin-4-yl)-2-(6-methyl-pyridin-2-yl)-imidazo[1,2-a]pyridine-6-carboxylic acid (50 mg, 0.144 mmol; see Example 55 for its preparation) was added with DMF (1 mL). The reaction was stirred until all material had dissolved. HATU (77 mg, 0.201 mmol) was then added making sure all material had dissolved. This was followed by the addition of DIEA (180 uL, 0.720 mmol). This mixture was allowed to stir for 10 min. 2-Thiophen-2-yl-ethylamine (21 uL, 0.173 mmol) was then added and the reaction was stirred for 2 h and checked by HPLC. The reaction was purified by preparative HPLC (5→40% CH3CN:H20 with 0.1% TFA). ¹H NMR (d₆-DMSO, 400 MHz): 2.52 (s, 3H), 3.14 (t, 2H, J=6.4 Hz), 3.58 (q, 2H, J=6.4 Hz), 6.92 (d, 1H, J=5.7 Hz), 7.00 (m, 3H), 7.39 (dd, 1H, J=1.3, 5.0 Hz), 7.44 (d, 1H, J=8.0 Hz), 7.54 (bs, 1H), 7.94 (m, 4H), 8.34 (d, 1H, J=6.0 Hz), 8.90 (dd, 1H, J=5.3, 5.7 Hz), 9.70 (s, 1H). MS (ESP+) 456.1 (M+1).

### EXAMPLE 65

### 3-(2-Amino-pyrimidin-4-yl)-2-(6-methyl-pyridin-2-yl)-imidazo[1,2-a]pyridine-6-carboxylic acid cyclopropylamide

In a vial, 3-(2-amino-pyrimidin-4-yl)-2-(6-methyl-pyridin-2-yl)-imidazo[1,2-a]pyridine-6-carboxylic acid (50 mg, 0.144 mmol; see Example 55 for its preparation) was added with DMF (1 mL). The reaction was stirred until all material had dissolved. HATU (77 mg, 0.201 mmol) was then added making sure all material had dissolved. This was followed by the addition of DIEA (180 uL, 0.720 mmol). This mixture was allowed to stir for 10 min. Cyclopropylamine (12 uL, 0.173 mmol) was then added and the reaction was stirred for 2 h and checked by HPLC. The reaction was purified by preparative HPLC (5→40% CH₃CN:H2O with 0.1% TFA). ¹H NMR (d₆-DMSO, 400 MHz): 0.65 (m, 2H), 0.78 (m, 2H), 2.51 (s, 3H), 2.92 (m, 1H), 6.94 (d, 1H, J=6.0 Hz), 7.45 (d, 1H, J=7.4 Hz), 7.73 (bs, 1H), 7.93 (m, 4H), 8.33 (d, 1H, J=6.3 Hz), 8.72 (d, 1H, J=4.0 Hz), 9.70 (s, 1H). MS (ESP+) 386.4 (M+1).

### EXAMPLE 66

### 4-[6-Methyl-2-(6-methyl-pyridin-2-yl)-8-morpholin-4-yl-imidazo[1,2-a]pyridin-3-yl]-pyrimidin-2-ol

A mixture of 4-[8-bromo-6-methyl-2-(6-methyl-pyridin-2-yl)-imidazo[1,2-a]pyridin-3-yl]-pyrimidin-2-ol (30 mg, 0.08 mmol; see Example 67 for its preparation), morpholine (15 ul, 0.17 mmol), Pd(OAc)₂ (9 mg, 0.04 mmole), NaOtBu (19 mg, 0.198) and 2-dicyclohexylphosphino-2′-(N,N-dimethylamino)biphenyl (20 mg, 0.05 mmol) in dioxane (600 ul) was degassed under N₂. The reaction was heated (105° C.) for 1.5 h. The mixture was diluted with CH₂Cl₂ (2 mL) and filtered through celite. The solvent was removed invacuo. The residue was purified by HPLC (C18, H₂O:MeCN gradient) to afford the titled compound as a red solid (3.1 mg, 10%) ¹H NMR (300 MHz, CDCl₃) 8.82 (s, J=6.6 Hz 1H), 8.50 (d, J=8.1 Hz, 1H), 8.19 (s, 1H), 7.80 (t, J=8.1 Hz, 1H), 7.52(d, J=7.4 Hz, 1H), 7.03 (d, J=6.9 Hz, 1H) 6.69 (s, 1H) 4.05(m, 4H), 3.65 (m, 4H) 3.03 (s, 3H), 2.47(s, 3H); MS (ESP+) 403 (M+1).

### EXAMPLE 67

### 4-[8-Bromo-6-methyl-2-(6-methyl-pyridin-2-yl)-imidazo[1,2-a]pyridin-3-yl]-pyrimidin-2-ol

A mixture of 8-bromo-3-(2-methanesulfonyl-pyrimidin-4-yl)-6-methyl-2-(6-methyl-pyridin-2-yl)-imidazo[1,2-a]pyridine (877 mg, 1.91 mmol; prepared in accordance with Ex. 2), NH₄OAc (3 g), H₂O (3 mL and dioxane (10 mL) in DMSO (11 mL) was refluxed (12 h). The solvent was removed in vacuo. The residue was triturated with water to afford the titled compound as a yellow solid (682 mg, 90%). ¹H NMR (300 MHz, DSMO-d₆) 9.30 (s, 1H), 7.84 (m, 5H), 7.33 (d, J=7.0 Hz, 1H), 6.31 (d, J=6.0 Hz, 1H), 2.45 (s, 3H), 2.37 (s, 3H); MS (ESP+) 396 (M+1).

### EXAMPLE 68

### 4-[6-Methyl-2-(6-methyl-pyridin-2-yl)-8-(2-pyridin-3-yl-ethylamino)-imidazo[1,2-a]pyridin-3-yl]-pyrimidin-2-ol

A mixture of 4-[8-bromo-6-methyl-2-(6-methyl-pyridin-2-yl)-imidazo[1,2-a]pyridin-3-yl]-pyrimidin-2-ol (50 mg, 0.13 mmol; see Example 67 for its preparation), 2-pyridin-3-yl-ethylamine (34 mg, 0.28 mmol), Pd(OAc)₂ (15 mg, 0.07 mmol), NaOtBu (32 mg, 0.33 mmol) and 2-dicyclohexylphosphino-2′-(N,N-dimethylamino)biphenyl (33 mg, 0.084 mmol) in dioxane (1 mL) was degassed under N₂. The reaction was heated (160° C.) in a microwave for 30 min. The mixture was diluted with CH₂Cl₂ (2 mL) and MeOH (100 ul) and passed through a plug of SiO₂. The residue was purified by HPLC (C18, H₂O:MeCN gradient (10 mM NH₄HCO₃ buffer)) to afford the titled compound as a red solid (5 mg, 9%). MS (ESP+) 438 (M+1).

### EXAMPLE 69

### 4-[6-Methyl-2-(6-methyl-pyridin-2-yl)-8-(2-pyridin-2-yl-ethylamino)-imidazo[1,2-a]pyridin-3-yl]-pyrimidin-2-ol

A mixture of 4-[8-bromo-6-methyl-2-(6-methyl-pyridin-2-yl)-imidazo[1,2-a]pyridin-3-yl]-pyrimidin-2-ol (50 mg, 0.13 mmol; see Example 67 for its preparation), 2-pyridin-2-yl-ethylamine (34 mg, 0.28 mmol), Pd(OAc)₂ (15 mg, 0.07 mmol), NaOtBu (32 mg, 0.33 mmol) and 2-dicyclohexylphosphino-2′-(N,N-dimethylamino)biphenyl (33 mg, 0.084 mmol) in dioxane (1 mL) was degassed under N₂. The reaction was heated (160° C.) in a microwave for 30 min. The mixture was diluted with CH₂Cl₂ (2 mL) and MeOH (100 ul) and passed through a plug of SiO₂. The residue was purified by HPLC (C18, H₂O:MeCN gradient (10 mM NH₄HCO₃ buffer)) to afford the titled compound as yellow solid (3 mg, 5%). ¹H NMR (300 MHz, CDCl₃) 8.63 (s, J=4.2 Hz 1H), 7.79 (d, J=7.9 Hz, 1H), 7.64 (td, J 7.9, 2.0 Hz, 1H), 7.22 (m, 2H), 6.61 (s, 1H), 6.23 (s, 1H), 5.70 (s, 1H), 5.32 (s, 1H), 3.73 (q, J=6.4 Hz, 2H), 3.24 (t, J=6.4 Hz, 2H), 2.87 (s, 3H), 2.35 (s, 3H); MS (ESP+) 438 (M+1).

### EXAMPLE 70

### 4-[6-Methyl-2-(6-methyl-pyridin-2-yl)-8-(2-pyridin-4-yl-ethylamino)-imidazo[1,2-a]pyridin-3-yl]-pyrimidin-2-ol

A mixture of 4-[8-bromo-6-methyl-2-(6-methyl-pyridin-2-yl)-imidazo[1,2-a]pyridin-3-yl]-pyrimidin-2-ol (50 mg, 0.13 mmol; see Example 67 for its preparation), 2-pyridin-4-yl-ethylamine (34 mg, 0.28 mmol), Pd(OAc)₂ (15 mg, 0.07 mmol), NaOtBu (32 mg, 0.33 mmol) and 2-dicyclohexylphosphino-2′-(N,N-dimethylamino)biphenyl (33 mg, 0.084 mmol) in dioxane (1 mL) was degassed under N₂. The reaction was heated (160° C.) in a microwave for 30 min. The mixture was diluted with CH₂Cl₂ (2 mL) and MeOH (100 ul) and passed through a plug of SiO₂. The residue was purified by HPLC (C18, H₂O:MeCN gradient (10 mM NH₄HCO₃ buffer)) to afford the titled compound as yellow solid (2 mg, 4%). ¹H NMR (300 MHz, CDCl₃) 8.57 (m, 2H), 7.9 (s, 1H), 7.79 (t, J=7.6 Hz, 1H), 7.23 (m, 3H), 6.59 (d, J=6.0, 1H), 6.21 (s, 1H), 5.39 (t, J=6.1 Hz, 1H), 3.64 (q, J=7.0 Hz, 2H), 3.08 (t, J=7.0 Hz, 2H), 2.84 (s, 3H), 2.37 (s, 3H); MS (ESP+) 438 (M+1).

### EXAMPLE 71

### 4-[6-Methyl-2-(6-methyl-pyridin-2-yl)-8-(2-morpholin-4-yl-ethylamino)-imidazo[1,2-a]pyridin-3-yl]-pyrimidin-2-ol

A mixture of 4-[8-bromo-6-methyl-2-(6-methyl-pyridin-2-yl)-imidazo[1,2-a]pyridin-3-yl]-pyrimidin-2-ol (50 mg, 0.13 mmol; see Example 67 for its preparation), 2-morpholin-4-yl-ethylamine (36 mg, 0.28 mmol), Pd(OAc)₂ (15 mg, 0.07 mmol), NaOtBu (32 mg, 0.33 mmol) and 2-dicyclohexylphosphino-2′-(N,N-dimethylamino)biphenyl (33 mg, 0.084 mmol) in dioxane (1 mL) was degassed under N₂. The reaction was heated (160° C.) in a microwave for 30 min. The mixture was diluted with CH₂Cl₂ (2 mL) and MeOH (100 ul) and passed through a plug of SiO₂. The residue was purified by HPLC (C18, H₂O:MeCN gradient (10 mM NH₄HCO₃ buffer)) to afford the titled compound as yellow solid (4.5 mg, 8%). ¹H NMR (300 MHz, CDCl₃) 8.11 (br.s, 1H), 7.80 (t, J=7.7 Hz, 1H), 7.26 (d, J=8.4 Hz, 1H), 6.61 (d, J=6.50, 1H), 6.22 (s, 1H), 3.84 (m, 4H), 3.48 (t, J=5.5 Hz, 2H), 2.89 (t, J=6.1 Hz, 2H), 2.86 (s, 3H), 2.69 (s, 4H) 2.36(s 3H); MS (ESP+) 446 (M+1).

### EXAMPLE 72

### [3-(2-Amino-pyrimidin-4-yl)-6-methyl-2-(6-methyl-pyridin-2-yl)-imidazo[1,2-a]pyridin-8-yl]-(3-morpholin-4-yl-propyl)-amine

A mixture of 4-[8-bromo-6-methyl-2-(6-methyl-pyridin-2-yl)-imidazo[1,2-a]pyridin-3-yl]-pyrimidin-2-ylamine (50 mg, 0.13 mmol), 3-morpholin-4-yl-propylamine (35 mg, 0.28 mmol), Pd(OAc)₂ (15 mg, 0.07 mmol), NaOtBu (32 mg, 0.33 mmol), H₂O (5 μL) and 2-dicyclohexylphosphino-2′-(N,N-dimethylamino)biphenyl (33 mg, 0.084 mmol) in dioxane (1 mL) was degassed under N₂. The reaction was heated (160° C.) for 1 h. The reaction was filtered through celite. The residue was purified by HPLC (C18, H₂O:MeCN gradient (10 mM NH₄HCO₃ buffer)) to afford the titled compound as yellow solid (5 mg, 8%). ¹H NMR (300 MHz, CDCl₃) 8.42 (s, 1H), 8.14 (d, J=5.1 Hz, 1H), 7.63 (t, J=7.8 Hz, 1H), 7.55 (d, J=7.1 Hz, 1H), 7.14 (d, J=7.8 Hz, 1H), 6.67 (d, J=5.1 Hz, 1H), 6.12 (s, 1H), 5.79 (t, J=5.5 Hz, 1H), 5.11 (s, 1H), 3.8 (m, 4H), 3.36 (q, J=6.3 Hz, 2H), 2.58 (s, 3H), 2.53 (t, J=7.1 Hz, 2H), 2.49 (m, 4H), 2.31 (s, 3H), 1.92 (quint, J=6.7 Hz, 2H); MS (ESP+) 459 (M+1).

### EXAMPLE 73

### [3-(2-Amino-pyrimidin-4-yl)-6-methyl-2-(6-methyl-pyridin-2-yl)-imidazo[1,2-a]pyridin-8-yl]-(2-morpholin-4-yl-ethyl)-amine

A mixture of 4-[8-bromo-6-methyl-2-(6-methyl-pyridin-2-yl)-imidazo[1,2-a]pyridin-3-yl]-pyrimidin-2-ylamine (50 mg, 0.13 mmol), 2-morpholin-4-yl-ethylamine (32 mg, 0.28 mmol), Pd(OAc)₂ (15 mg, 0.07 mmol), NaOtBu (32 mg, 0.33 mmol), H₂O (5 μL) and 2-dicyclohexylphosphino-2′-(N,N-dimethylamino)biphenyl (33 mg, 0.084 mmol) in dioxane (1 mL) was degassed under N₂. The reaction was heated (160° C.) for 1 h. The reaction was filtered through celite. The residue was purified by HPLC (C18, H₂O:MeCN gradient (10 mM NH₄HCO₃ buffer)) to afford the titled compound as red solid (6 mg, 10%). ¹H NMR (300 MHz, CDCl₃) 8.44 (s, 1H), 8.15 (d, J=5.0 Hz, 1H), 7.63 (t, J=7.6 Hz, 1H), 7.55 (d, J=7.4 Hz, 1H), 7.15 (d, J=7.7 Hz, 1H), 6.66 (d, J=5.5 Hz, 1H), 6.12 (s, 1H), 5.57 (t, J=5.3 Hz, 1H), 5.11 (s, 1H), 3.76 (m, 4H), 3.37 (q, J=6.1 Hz, 2H), 2.74 (t, J=6.3 Hz, 2H), 2.59 (s, 3H), 2.54 (m, 4H), 2.31 (s, 3H); MS (ESP+) 445 (M+1).

### EXAMPLE 74

### 4-[6-Methyl-2-(6-methyl-pyridin-2-yl)-8-morpholin-4-yl-imidazo[1,2-a]pyridin-3-yl]-pyrimidin-2-ylamine

A mixture of 4-[8-bromo-6-methyl-2-(6-methyl-pyridin-2-yl)-imidazo[1,2-a]pyridin-3-yl]-pyrimidin-2-ylamine (50 mg, 0.13 mmol), morpholine (25 μL, 0.29 mmol), Pd(OAc)₂ (15 mg, 0.07 mmol), NaOtBu (32 mg, 0.33 mmol), H₂O (5 μL) and 2-dicyclohexylphosphino-2′-(N,N-dimethylamino)biphenyl (33 mg, 0.084 mmol) in dioxane (1 mL) was degassed under N₂. The reaction was heated (160° C.) for 1 h. The reaction was filtered through celite. The residue was purified by HPLC (C18, H₂O:MeCN gradient (10 mM NH₄HCO₃ buffer)) to afford the titled compound as red solid (2.9 mg, 6%). ¹H NMR (300 MHz, CDCl₃) 8.70 (s, 1H), 8.15 (d, J=5.5 Hz, 1H), 7.79 (d, J=7.8 Hz, 1H), 7.67 (t, J=7.8 Hz, 1H), 7.14 (d, J=7.6 Hz, 1H), 6.84 (d, J=5.5 Hz, 1H), 6.46 (s, 1), 5.23 (s, 2H), 4.01 (m, 4H), 3.59 (m, 4H), 2.5 (s, 3H), 2.35 (s, 3H); MS (ESP+) 402 (M+1).

### EXAMPLE 75

### [3-(2-Amino-pyrimidin-4-yl)-6-methyl-2-(6-methyl-pyridin-2-yl)-imidazo[1,2-a]pyridin-8-yl]-(2-pyridin-3-yl-ethyl)-amine

A mixture of 4-[8-bromo-6-methyl-2-(6-methyl-pyridin-2-yl)-imidazo[1,2-a]pyridin-3-yl]-pyrimidin-2-ylamine (50 mg, 0.13 mmol), 2-pyridin-3-yl-ethylamine (33 mg, 0.29 mmol), Pd(OAc)₂ (15 mg, 0.07 mmol), NaOtBu (32 mg, 0.33 mmol), H₂O (5 μL) and 2-dicyclohexylphosphino-2′-(N,N-dimethylamino)biphenyl (33 mg, 0.084 mmol) in dioxane (1 mL) was degassed under N₂. The reaction was heated (160° C.) for 1 h. The reaction was filtered through celite. The residue was purified by HPLC (C18, H₂O:MeCN gradient (10 mM NH₄HCO₃ buffer)) to afford the titled compound as red solid (6.5 mg, 11%). ¹H NMR (300 MHz, CDCl₃) 8.57 (d, J=2.1, 1H), 8.51 (dd, J=4.8, 1.6 Hz, 1H), 8.46 (s, 1H), 8.12 (d, J=5.4 Hz, 1H), 7.63 (m, 2H), 7.56 (d, J=7.5 Hz, 1H), 7.26 (dd, J=8.1, 4.8 Hz, 1H), 7.16 (d, J=7.5 Hz, 1H), 6.67 (d, J=5.4 Hz, 1H), 6.14 (s, 1H), 5.50 (s, 1H), 5.37 (s, 2H), 3.57 (q, J=7.5 Hz, 2H), 3.05 (t, J=7.5 Hz, 2H), 2.59 (s, 3H), 2.32 (s, 3H); MS (ESP+) 437 (M+1).

### EXAMPLE 76

### [3-(2-Amino-pyrimidin-4-yl)-6-methyl-2-(6-methyl-pyridin-2-yl)-imidazo[1,2-a]pyridin-8-yl]-(2-pyridin-2-yl-ethyl)-amine

A mixture of 4-[8-bromo-6-methyl-2-(6-methyl-pyridin-2-yl)-imidazo[1,2-a]pyridin-3-yl]-pyrimidin-2-ylamine (50 mg, 0.13 mmol), 2-pyridin-2-yl-ethylamine (33 mg, 0.29 mmol), Pd(OAc)₂ (15 mg, 0.07 mmol), NaOtBu (32 mg, 0.33 mmol), H₂O (5 μL) and 2-dicyclohexylphosphino-2′-(N,N-dimethylamino)biphenyl (33 mg, 0.084 mmol) in dioxane (1 mL) was degassed under N₂. The reaction was heated (160° C.) for 1 h. The reaction was filtered through celite. The residue was purified by HPLC (C18, H₂O:MeCN gradient (10 mM NH₄HCO₃ buffer)) to afford the titled compound as red solid (2.6 mg, 5%). ¹H NMR (300 MHz, CDCl₃) 8.61 (d, J=4.9, 1H), 8.46 (s, 1H), 8.06 (d, J=6 Hz, 1H), 7.66 (m, 3H), 7.18 (m, 2H), 6.71 (d, J=6.1 Hz, 1H), 6.28 (s, 1H), 5.80 (s, 2H), 3.75 (t, J=7.3 Hz, 2H), 3.25 (t, J=7.3 Hz, 2H), 2.58 (s, 3H), 2.33 (s, 3H); MS (ESP+) 437 (M+1).

### EXAMPLE 77

### (3-(2-Amino-pyrimidin-4-yl)-6-methyl-2-(6-methyl-pyridin-2-yl)-imidazo[1,2-a]pyridin-8-yl]-(2-pyridin-4-yl-ethyl)-amine

A mixture of 4-[8-bromo-6-methyl-2-(6-methyl-pyridin-2-yl)-imidazo[1,2-a]pyridin-3-yl]-pyrimidin-2-ylamine (50 mg, 0.13 mmol), 2-pyridin-4-yl-ethylamine (33 mg, 0.29 mmol), Pd(OAc)₂ (15 mg, 0.07 mmol), NaOtBu (32 mg, 0.33 mmol), H₂O (5 μL) and 2-dicyclohexylphosphino-2′-(N,N-dimethylamino)biphenyl (33 mg, 0.084 mmol) in dioxane (1 mL) was degassed under N₂. The reaction was heated (160° C.) for 1 h. The reaction was filtered through celite. The residue was purified by HPLC (C18, H₂O:MeCN gradient (10 mM NH₄HCO₃ buffer)) to afford the titled compound as red solid (6 mg, 11%). ¹H NMR (300 MHz, CDCl₃) 8.55 (m, 2H), 8.46 (s, 1H), 8.11 (d, J=6 Hz, 1H), 7.66 (t, J=7.8 Hz, 1H), 7.58 (d, J=8.4 Hz, 1H), 7.23 (m, 2H), 7.18 (d, J=7.8 Hz, 1H), 6.67 (d, J=5.4 Hz, 1H), 6.17 (s, 1H), 5.6 (s, 1H), 5.46 (s, 2H), 3.59 (q, J=6.6 Hz, 2H), 3.06 (t, J=7.2 Hz, 211), 2.58 (s, 3H), 2.33 (s, 3H); MS (ESP+) 437 (M+1).

### EXAMPLE 78

### 2-(6-Methyl-pyridin-2-yl)-3-(2-methylsulfanyl-pyrimidin-4-yl)-imidazo[1,2-a]pyridine-7-carboxylic acid ethyl ester

Hydrogen bromide/acetic acid (30 wt %, 40.11 mmol) and 1.00 M bromine/acetic acid (8.02 mmol) was added to a solution of 1-(6-methyl-pyridin-2-yl)-2-(2-methylsulfanyl-pyrimidin-4-yl)-ethanone (6.17 mmol; see Example 1(b) for its preparation) and catalytic BHT in glacial acetic acid (29.6 mL) at RT to form a precipitate. After 0.3 h the reaction was diluted to 400 mL with ether, filtered, washed with ether, dried briefly under N₂ gas to give an orange solid. The solid was added to a flask containing 2-amino-isonicotinic acid ethyl ester (6.17 mmol). Toluene (20 mL) was added and the slurry was heated to 100° C. Diisopropylethylamine (24.68 mmol) was added dropwise. After 3.3 h the reaction was allowed to cool to 0° C. and precipitate was formed. The slurry was filtered, the solid washed with acetonitrile and air dried to give 1.160 g of a solid identified as 2-(6-methyl-pyridin-2-yl)-3-(2-methylsulfanyl-pyrimidin-4-yl)-imidazo[1,2-a]pyridine-7-carboxylic acid ethyl ester. ¹H NMR (CDCl₃, 300 MHz): 1.44 (t, J=7.4, 3H), 2.54 (s, 3H), 2.64 (s, 3H), 4.49 (q, J=7.0, 2H), 7.21 (dd, J=7.4, 1.3 Hz, 1H), 7.23 (d, J=5.4 Hz, 1H), 7.54 (dd, J=7.4, 1.4 Hz, 1H), 7.73 (m, 2H), 8.41 (d, J=5.4 Hz, 1H), 8.45 (dd, J=1.7, 0.8 Hz, 1H), 9.46 (dd, J=7.4, 0.8 Hz, 1H); MS (ESP+) 406.4 (M+1).

### EXAMPLE 79

### 3-(2-Methanesulfonyl-pyrimidin-4-yl)-2-(6-methyl-pyridin-2-yl)-imidazo[1,2-a]pyridine-7-carboxylic acid ethyl ester

4.0 N Sulfuric acid (0.33 mmol), catalytic sodium tungstate dihydrate, and 30 wt % hydrogen peroxide (9.44 mmol) were added to a slurry of 2-(6-methyl-pyridin-2-yl)-3-(2-methylsulfanyl-pyrimidin-4-yl)-imidazo[1,2-a]pyridine-7-carboxylic acid ethyl ester (2.86 mmol; see Example 78 for its preparation) in 1:1 ethanol/dichloromethane (25 mL) at 55° C. After 5.25 h, the reaction was diluted with water (25 mL) and warmed at 55° C. for an additional 1 h. The reaction was then cooled to RT, quenched with saturated sodium thiosulfate and then extracted with dichloromethane (300 mL). The organic phase was washed with saturated sodium bicarbonate (125 mL) and brine (125 mL), dried (Na₂SO₄) and concentrated in vacuo to give 1.264 g of a solid identified as 3-(2-methanesulfonyl-pyrimidin-4-yl)-2-(6-methyl-pyridin-2-yl)-imidazo[1,2-a]pyridine-7-carboxylic acid ethyl ester. ¹H NMR (CDCl₃, 300 MHz): 1.45 (t, J=7.2 Hz, 3H), 2.53 (s, 3H), 3.40 (s, 3H), 4.46 (q, J=7.2 Hz, 2H), 7.26 (d, J=7.6 Hz, 1H), 7.67 (dd, J=7.4, 1.8 Hz, 1H), 7.78 (t, J=7.6 Hz, 1H), 7.91 (d, J=7.6 Hz, 1H), 8.00, (d, J=5.4 Hz, 1H), 8.48 (m, 1H), 8.72 (d, J=5.4 Hz, 1H), 9.70 (d, J=7.4 Hz, 1H); MS (ESP+) 438.4 (M+1).

### EXAMPLE 80

### 3-(2-Amino-pyrimidin-4-yl)-2-(6-methyl-pyridin-2-yl)-imidazo[1,2-a]pyridine-7-carboxylic acid ethyl ester

A solution of 3-(2-methanesulfonyl-pyrimidin-4-yl)-2-(6-methyl-pyridin-2-yl)-imidazo[1,2-a]pyridine-7-carboxylic acid ethyl ester (6.35 mmol; see Example 79 for its preparation) and ammonium acetate (190.50 mmol) in 2:1 dioxane/water (84 mL) was warmed to 100° C. for 5 days. The reaction solution was then concentrated in vacuo, diluted with water (˜100 mL) to give a precipitate that was filtered, washed with water and air dried to give 2.035 g of a solid identified as 3-(2-amino-pyrimidin-4-yl)-2-(6-methyl-pyridin-2-yl)-imidazo[1,2-a]pyridine-7-carboxylic acid ethyl ester. ¹H NMR (CDCl₃, 300 MHz): 1.44 (t, J=7.2 Hz, 3H), 2.56 (s, 3H), 4.44 (q, J=7.2 Hz, 2H), 6.81 (d, J=5.4 Hz, 1H), 7.19 (m, 1H), 7.50 (d, J=7.3 Hz, 1H), 7.68 (m, 2H), 8.17 (d, J=5.4 Hz, 1H), 8.44 (m, 1H), 9.40 (d, J=7.3 Hz, 1H); MS (ESP+) 375.3 (M+1).

### EXAMPLE 81

### 3-(2-Amino-pyrimidin-4-yl)-2-(6-methyl-pyridin-2-yl)-imidazo[1,2-a]pyridine-7-carboxylic acid

Lithium hydroxide monohydrate (0.967 mmol) was added to a solution of 3-(2-amino-pyrimidin-4-yl)-2-(6-methyl-pyridin-2-yl)-imidazo[1,2-a]pyridine-7-carboxylic acid ethyl ester (0.134 mmol; see Example 80 for its preparation) in 2:1 tetrahydrofuran/water (2.7 mL). After 2 h, the reaction was concentrated in vacuo to remove the organic phase, diluted with water (˜3 mL) and acidified to pH 5 with 10% HCl. The reaction was cooled to 0° C. to give a precipitate that was filtered, washed with water and air dried. The resulting solid was purified via reverse phase HPLC (acetonitrile/water gradient with 0.1% TFA) to give 33 mg of a yellow solid identified as the tri-TFA salt of 3-(2-amino-pyrimidin-4-yl)-2-(6-methyl-pyridin-2-yl)-imidazo[1,2-a]pyridine-7-carboxylic acid. ¹H (d6-DMSO, 300 MHz): 2.42 (s, 3H), 6.59 (d, J=5.3 Hz, 1H), 6.76 (s, 2H), 7.27 (d, J=7.3 Hz, 1H), 7.46 (dd, J=7.3, 1.7 Hz, 1H, 7.73 (d, J=7.8 Hz, 1H), 7.81 (t, J=7.8 Hz, 1H), 8.06 (m, 1H), 8.16 (d, J=5.3 Hz, 1H), 9.37 (d, J=7.2 Hz, 1H); MS (ESP+) 347.5 (M+1).

### EXAMPLE 82

### N-[3-(2-Amino-pyrimidin-4-yl)-2-(6-methyl-pyridin-2-yl)-imidazo[1,2-a]pyridine-7-carbonyl]-methanesulfonamide

HATU (0.405 mmol), diisopropylethylamine (1.445 mmol) and methanesulfonamide (0.347 mmol) were added to a slurry of 3-(2-amino-pyrimidin-4-yl)-2-(6-methyl-pyridin-2-yl)-imidazo[1,2-a]pyridine-7-carboxylic acid (0.289 mmol; see Example 81 for its preparation) in N,N-dimethylformamide (2.9 mL) at RT. After 0.7 h, HATU (0.405 mmol), diisopropylethylamine (1.445 mmol) and methanesulfonamide (0.347 mmol) were added and the reaction was stirred at RT for an additional 2.75 h. The reaction was concentrated in vacuo and purified via reverse phase HPLC (acetonitrile/water gradient with 0.1% TFA) to give 65 mg of a yellow solid identified as the tri-TFA salt of N-[3-(2-amino-pyrimidin-4-yl)-2-(6-methyl-pyridin-2-yl)-imidazo[1,2-a]pyridine-7-carbonyl]-methanesulfonamide. ¹H (d6-DMSO, 300 MHz): 3.44 (s, 3H), 6.82 (d, J=6.0 Hz, 1H), 7.44 (d, J=7.8 Hz, 1H), 7.56 (dd, J=7.4, 1.8 Hz, 1H), 7.82 (bs, 2H), 7.86 (d, J=7.8 Hz, 1H), 7.95 (t, J=7.8 Hz, 1H), 8.25 (d, J=6.0 Hz, 1H), 8.49 (m, 1H), 9.62 (d, J=7.4 Hz, 1H); MS (ESP+) 424.11 (M+1); MS (ESP−) 422.14 (M−1).

### EXAMPLE 83

### 3-(2-Amino-pyrimidin-4-yl-2-(6-methyl-pyridin-2-yl)-imidazo[1,2-a]pyridine-7-carboxylic acid cyclopropylamide

HATU (0.304 mmol), diisopropylethylamine (1.085 mmol) and cyclopropylamine (0.260 mmol) were added to a slurry of 3-(2-amino-pyrimidin-4-yl)-2-(6-methyl-pyridin-2-yl)-imidazo[1,2-a]pyridine-7-carboxylic acid (0.217 mmol; see Example 81 for its preparation) in N,N-dimethylformamide (2.2 mL) at RT. After 3.25 h, the reaction was concentrated in vacuo and purified via reverse phase HPLC (acetonitrile/water gradient with 0.1% TFA) to give 81 mg of a yellow solid identified as the tri-TFA salt of 3-(2-amino-pyrimidin-4-yl)-2-(6-methyl-pyridin-2-yl)-imidazo[1,2-a]pyridine-7-carboxylic acid cyclopropylamide. ¹H (d6-DMSO, 300 MHz): 0.64 (m, 2H), 0.75 (m, 2H), 2.50 (s, 3H), 2.91 (m, 1H), 6.82 (d, J=6.1 Hz, 1H), 7.20 (bs, 2H), 7.43 (d, J=7.7 Hz, 1H), 7.54 (dd, J=7.5, 1.6 Hz, 1H), 7.83 (d, J=7.4 Hz, 1H), 7.94 (t, J=7.7 Hz, 1H), 8.22 (d, J=6.2 Hz, 1H), 8.29 (m, 1H), 8.82 (d, J=4.0 Hz, 1H), 9.62 (d, J=7.4 Hz, 1H); MS (ESP+) 386.5 (M+1).

### EXAMPLE 84

### 3-(2-Amino-pyrimidin-4-yl)-2-(6-methyl-pyridin-2-yl)-imidazo[1,2-a]pyridine-7-carboxylic acid (2-thiophen-2-yl-ethyl)-amide

HATU (0.202 mmol), diisopropylethylamine (0.720 mmol) and 2-thiopheneethylamine (0.173 mmol) were added to a slurry of 3-(2-amino-pyrimidin-4-yl)-2-(6-methyl-pyridin-2-yl)-imidazo[1,2-a]pyridine-7-carboxylic acid (0.144 mmol; see Example 81 for its preparation) in N,N-dimethylformamide (1.4 mL) at RT. After 18 h, the reaction was concentrated in vacuo and purified via reverse phase HPLC (acetonitrile/water gradient with 0.1% TFA) to give 81 mg of a yellow solid identified as the tri-TFA salt of 3-(2-amino-pyrimidin-4-yl)-2-(6-methyl-pyridin-2-yl)-imidazo[1,2-a]pyridine-7-carboxylic acid (2-thiophen-2-yl-ethyl)-amide. ¹H (d6-DMSO, 300 MHz): 2.50 (s, 3H), 3.13 (t, J=6.6 Hz, 2H), 3.57 (m, 2H), 6.83 (d, J=6.1 Hz, 1H), 6.96 (m, 2H), 7.36 (m, 1H), 7.44 (d, J=7.8 Hz, 1H), 7.55 (d, J=7.4 Hz, 1H), 7.85 (d, J=7.9 Hz, 1H), 7.96 (t, J=7.9 Hz, 1H), 8.23 (d, J=6.2 Hz, 1H), 8.30 (m, 1H), 9.04 (m, 1H), 9.61 (d, J=7.3 Hz, 1H); MS (ESP+) 456.22 (M+1).

### EXAMPLE 85

### 3-(2-Amino-pyrimidin-4-yl)-2-(6-methyl-pyridin-2-yl)-imidazo[1,2-a]pyridine-7-carboxylic acid ethylamide

HATU (0.202 mmol), diisopropylethylamine (0.720 mmol) and ethylamine (0.173 mmol) were added to a slurry of 3-(2-amino-pyrimidin-4-yl)-2-(6-methyl-pyridin-2-yl)-imidazo[1,2-a]pyridine-7-carboxylic acid (0.144 mmol; see Example 81 for its preparation) in N,N-dimethylformamide (1.4 mL) at RT. After 20 h, the reaction was concentrated in vacuo and purified via reverse phase HPLC (acetonitrile/water gradient with 0.1% TFA) to give 59 mg of a yellow solid identified as the tri-TFA salt of 3-(2-amino-pyrimidin-4-yl)-2-(6-methyl-pyridin-2-yl)-imidazo[1,2-a]pyridine-7-carboxylic acid ethylamide. ¹H (d6-DMSO, 300 MHz): 1.18 (t, J=7.2 Hz, 3H), 2.50 (s, 3H), 3.36 (m, 2H), 6.84 (d, J=6.2 Hz, 1H), 7.45 (d, J=7.8 Hz, 1H), 7.57 (dd, J=7.4, 1.9 Hz, 1H), 7.85 (d, J=7.8 Hz, 1H), 7.96 (t, J=7.8 Hz, 1H), 8.22 (d, J=6.2 Hz, 1H), 8.31 (m, 1H), 8.88 (t, J=5.6 Hz, 1H), 9.65 (d, J=7.4 Hz, 1H); MS (ESP+) 374.21 (I+1).

### EXAMPLE 86

### 3-(2-Amino-pyrimidin-4-yl)-2-(6-methyl-pyridin-2-yl)-imidazo[1,2-a]pyridine-7-carboxylic acid methoxy-amide

HATU (0.202 mmol), diisopropylethylamine (1.008 mmol) and methoxylamine hydrochloride (0.173 mmol) were added to a slurry of 3-(2-amino-pyrimidin-4-yl)-2-(6-methyl-pyridin-2-yl)-imidazo[1,2-a]pyridine-7-carboxylic acid (0.144 mmol; see Example 81 for its preparation) in N,N-dimethylformamide (1.4 mL) at RT. After 20 h, the reaction was concentrated in vacuo and purified via reverse phase HPLC (acetonitrile/water gradient with 0.1% TFA) to give 59 mg of a yellow solid identified as the tri-TFA salt of 3-(2-amino-pyrimidin-4-yl)-2-(6-methyl-pyridin-2-yl)-imidazo[1,2-a]pyridine-7-carboxylic acid methoxy-amide. ¹H (d6-DMSO, 300 MHz): 2.50 (s, 3H), 3.78 (s, 3H), 6.78 (d, J=5.9 Hz, 1H), 7.41 (m, 2H), 7.55 (bs, 2H), 7.82 (d, J=7.5 Hz, 1H), 7.92 (t, J=7.8 Hz, 1H), 8.16 (m, 1H), 8.22 (d, J=5.9 Hz, 1H), 9.59 (d, J=7.8 Hz, 1H), 12.15 (bs, 1H); MS (ESP+) 376.17 (M+1).

### EXAMPLE 87

### 3-(2-Amino-pyrimidin-4-yl)-2-(6-methyl-pyridin-2-yl)-imidazo[1,2-a]pyridine-7-carboxylic acid (2-amino-ethyl)-amide

HATU (0.202 mmol), diisopropylethylamine (0.720 mmol) and N-(2-aminoethyl)carbamic acid tert-butyl ester (0.173 mmol) were added to a slurry of 3-(2-amino-pyrimidin-4-yl)-2-(6-methyl-pyridin-2-yl)-imidazo[1,2-a]pyridine-7-carboxylic acid (0.144 mmol; see Example 81 for its preparation) in N,N-dimethylformamide (1.4 mL) at RT. After 18 h, the reaction was concentrated in vacuo and was then dissolved in 1:1 dichloromethane/TFA (1 mL) at RT. After 2 h, the reaction was concentrated in vacuo and purified via reverse phase HPLC (acetonitrile/water gradient with 0.1% TFA) to give 17 mg of a yellow solid identified as the tri-TFA salt of 3-(2-amino-pyrimidin-4-yl)-2-(6-methyl-pyridin-2-yl)-imidazo[1,2-a]pyridine-7-carboxylic acid (2-amino-ethyl)-amide. ¹H (d6-DMSO, 400 MHz): 2.46 (s, 3H), 3.05 (q, J=5.9 Hz, 21), 3.57 (dt, J=5.9, 5.9 Hz, 2H), 6.74 (d, J=5.8 Hz, 1H), 7.37 (d, J=7.8 Hz, 1H), 7.50 (dd, J=7.4, 1.8 Hz, 1H), 7.82 (d, J=7.6 Hz, 1H), 7.85 (bs, 2H), 7.89 (d, J=7.7 Hz, 1H), 8.22 (d, J=5.8 Hz, 1H), 8.32 (dd, J=1.7, 0.8 Hz, M1), 8.97 (t, J=5.7 Hz, 1H), 9.58 (dd, J=7.4, 0.7 Hz, 1H); MS (ESP+) 389.21 (M+1).

### EXAMPLE 88

### 3-(2-Amino-pyrimidin-4-yl)-2-(6-methyl-pyridin-2-yl)-imidazo[1,2-a]pyridine-7-carboxylic acid (piperidin-3-ylmethyl)-amide

HATU (0.202 mmol), diisopropylethylamine (0.720 mmol) and (3-aminomethyl)-1-N-Boc-piperdine (0.173 mmol) were added to a slurry of 3-(2-amino-pyrimidin-4-yl)-2-(6-methyl-pyridin-2-yl)-imidazo[1,2-a]pyridine-7-carboxylic acid (0.144 mmol; see Example 81 for its preparation) in N,N-dimethylformamide (1.4 mL) at RT. After 18 h, the reaction was concentrated in vacuo and was then dissolved in 1:1 dichloromethane/TFA (1 mL) at RT. After 2 h, the reaction was concentrated in vacuo and purified via reverse phase HPLC (acetonitrile/water gradient with 0.1% TFA) to give 32 mg of a yellow solid identified as the tri-TFA salt of 3-(2-amino-pyrimidin-4-yl)-2-(6-methyl-pyridin-2-yl)-imidazo[1,2-a]pyridine-7-carboxylic acid (piperidin-3-ylmethyl)-amide. ¹H (d6-DMSO, 400 MHz): 1.37 (q, J=12.2 Hz, 2H), 1.86 (d, J=13.9 Hz, 3H), 2.46 (s, 3H), 2.87 (q, J=11.3 Hz, 2H), 3.28 (m, 4H), 6.73 (d, J=5.8 Hz, 1H), 7.27 (bs, 1H), 7.36 (d, J=7.8 Hz, 1H), 7.48 (dd, J=7.4, 1.8 Hz, 1H), 7.79 (d, J=7.8 Hz, 1H), 7.88 (t, J=7.8 Hz, 1H), 8.17 (bs, 1H), 8.21 (d, J=5.8 Hz, 1H), 8.29 (m, 1H), 8.50 (bs, 1H), 8.92 (t, J=6.0 Hz, 1H), 9.55 (d, J=7.4 Hz, 1H); MS (ESP+) 443.16 (M+1).

### EXAMPLE 89

### 3-(2-Amino-pyrimidin-4-yl)-2-(6-methyl-pyridin-2-yl)-imidazo[1,2-a]pyridine-7-carboxylic acid 2,2-dimethylhydrazide

HATU (0.202 mmol), diisopropylethylamine (0.720 mmol) and unsym-dimethylhydrazine (0.173 mmol) were added to a slurry of 3-(2-amino-pyrimidin-4-yl)-2-(6-methyl-pyridin-2-yl)-imidazo[1,2-a]pyridine-7-carboxylic acid (0.144 mmol; see Example 81 for its preparation) in N,N-dimethylformamide (1.4 mL) at RT. After 15 h, the reaction was concentrated in vacuo and purified via reverse phase HPLC (acetonitrile/water gradient with 0.1% TFA) to give 31 mg of a yellow solid identified as the tri-TFA salt of 3-(2-amino-pyrimidin-4-yl)-2-(6-methyl-pyridin-2-yl)-imidazo[1,2-a]pyridine-7-carboxylic acid 2,2-dimethylhydrazide. ¹H (d6-DMSO, 400 MHz): 2.50 (s, 3H), 2.71 (s, 6H), 6.81 (d, J=5.9 Hz, 1), 7.42 (d, J=7.8 Hz, 1H), 7.49 (d, J=7.5 Hz, 1H), 7.69 (bs, 2H), 7.83 (d, J=7.8 Hz, 1H), 7.94 (t, J=7.8 Hz, 1H), 8.22 (d, J=5.9 Hz, 1H), 8.26 (m, 1H), 9.62 (d, J=7.4 Hz, 1H), 10.10 (s, 1H); MS (ESP+) 389.16 (M+1).

### EXAMPLE 90

### 3-{[3-(2-Amino-pyrimidin-4-yl)-2-(6-methyl-pyridin-2-yl)-imidazo[1,2-a]pyridine-7-carbonyl]-amino}-propionic acid methyl ester

HATU (0.202 mmol), diisopropylethylamine (1.008 mmol) and 3-amino-propionic acid methyl ester hydrochloride (0.173 mmol) were added to a slurry of 3-(2-amino-pyrimidin-4-yl)-2-(6-methyl-pyridin-2-yl)-imidazo[1,2-a]pyridine-7-carboxylic acid (0.144 mmol; see Example 81 for its preparation) in N,N-dimethylformamide (1.4 mL) at RT. After 15 h, the reaction was concentrated in vacuo and purified via reverse phase HPLC (acetonitrile/water gradient with 0.1% TFA) to give 31 mg of a yellow solid identified as the tri-TFA salt of 3-{[3-(2-amino-pyrimidin-4-yl)-2-(6-methyl-pyridin-2-yl)-imidazo[1,2-a]pyridine-7-carbonyl]-amino}-propionic acid methyl ester. ¹H (d6-DMSO, 400 MHz): 2.50 (s, 3H), 2.66 (t, J=6.9 Hz, 21), 3.56 (dd, J=12.3, 6.7 Hz, 2H), 3.63 (s, 3H), 6.80 (d, J=5.9 Hz, 1H), 7.42 (d, J=7.7 Hz, 1H), 7.52 (dd, J=7.4, 1.8 Hz, 1H), 7.83 (d, J=7.7 Hz, 1H), 7.94 (t, J=7.7 Hz, 1H), 8.22 (d, J=5.9 Hz, 1H), 8.29 (dd, J=1.8, 0.8 Hz, 1H), 8.96 (t, J=5.4 Hz, 1H), 9.62 (d, J=7.4 Hz, 1H); MS (ESP+) 432.06 (M+1).

### EXAMPLE 91

### 3-(2-Amino-pyrimidin-4-yl)-2-(6-methyl-pyridin-2-yl)-imidazo[1,2-a]pyridine-7-carboxylic acid ([1,4]dioxan-2-ylmethyl)-amide

HATU (0.202 mmol), diisopropylethylamine (0.720 mmol) and C-[1,4]dioxan-2-ylmethylamine (0.173 mmol) were added to a slurry of 3-(2-amino-pyrimidin-4-yl)-2-(6-methyl-pyridin-2-yl)-imidazo[1,2-a]pyridine-7-carboxylic acid (0.144 mmol; see Example 81 for its preparation) in N,N-dimethylformamide (1.4 mL) at RT. After 15 h, the reaction was concentrated in vacuo and purified via reverse phase HPLC (acetonitrile/water gradient with 0.1% TFA) to give 31 mg of a yellow solid identified as the tri-TFA salt of 3-(2-amino-pyrimidin-4-yl)-2-(6-methyl-pyridin-2-yl)-imidazo[1,2-a]pyridine-7-carboxylic acid ([1,4]dioxan-2-ylmethyl)-amide. ¹H (d6-DMSO, 400 MHz): 2.50 (s, 3H), 3.28 (dd, J=11.3, 9.8 Hz, 1H), 3.35 (q, J=5.7 Hz, 2H), 3.52 (m, 2H), 3.65 (m, 1H), 3.71 (m, 1H), 3.78 (m, 2H), 6.81 (d, J=6.0 Hz, 1H), 7.42 (d, J=7.7 Hz, 1H), 7.55 (dd, J=7.4, 1.7 Hz, 1H), 7.83 (d, J=7.7 Hz, 114), 7.94 (t, J=7.7 Hz, 1H), 8.22 (d, J=6.0 Hz, 1H), 8.32 (m, 1H), 8.96 (t, J=5.8 Hz, 1H), 9.62 (d, J=7.4 Hz, 1H); MS (ESP+) 446.55 (M+1).

### EXAMPLE 92

### 3-(2-Amino-pyrimidin-4-yl)-2-(6-methyl-pyridin-2-yl)-imidazo[1,2-a]pyridine-7-carboxylic acid (2-hydroxy-ethyl)-amide

HATU (0.202 mmol), diisopropylethylamine (0.720 mmol) and ethanolamine (0.173 mmol) were added to a slurry of 3-(2-amino-pyrimidin-4-yl)-2-(6-methyl-pyridin-2-yl)-imidazo[1,2-a]pyridine-7-carboxylic acid (0.144 mmol; see Example 81 for its preparation) in N,N-dimethylformamide (1.4 mL) at RT. After 17.25 h, the reaction was concentrated in vacuo and purified via reverse phase HPLC (acetonitrile/water gradient with 0.1% TFA) to give 39 mg of a yellow solid identified as the tri-TFA salt of 3-(2-amino-pyrimidin-4-yl)-2-(6-methyl-pyridin-2-yl)-imidazo[1,2-a]pyridine-7-carboxylic acid (2-hydroxy-ethyl)-amide. ¹H (d6-DMSO, 400 MHz): 2.50 (s, 3H), 3.39 (dt, J=6.0, 6.0 Hz, 2H), 3.57 (t, J=6.2 Hz, 2H), 6.78 (d, J=6.0 Hz, 1H), 7.41 (d, J=7.8 Hz, 1H), 7.55 (dd, J=7.5, 1.8 Hz, 1H), 7.63, (bs, 2H), 7.83 (d, J=7.8 Hz, 1H), 7.93 (d, J=7.8 Hz, 1H), 8.21 (d, J=5.9 Hz, 1H), 8.32 (dd, J=1.7, 0.8 Hz, 1H), 8.86 (t, J=5.5 Hz, 1H), 9.61 (d, J=7.4 Hz, 1H); MS (ESP+) 390.48 (M+1).

### EXAMPLE 93

### 3-(2-Amino-pyrimidin-4-yl)-2-(6-methyl-pyridin-2-yl)-imidazo[1,2-a]pyridine-7-carboxylic acid (2-dimethylamino-ethyl)-amide

HATU (0.202 mmol), diisopropylethylamine (0.720 mmol) and N,N-dimethylethylenediamine (0.173 mmol) were added to a slurry of 3-(2-amino-pyrimidin-4-yl)-2-(6-methyl-pyridin-2-yl)-imidazo[1,2-a]pyridine-7-carboxylic acid (0.144 mmol; see Example 81 for its preparation) in N,N-dimethylformamide (1.4 mL) at RT. After 17.5 h, the reaction was concentrated in vacuo and purified via reverse phase HPLC (acetonitrile/water gradient with 0.1% TFA) to give 28 mg of a yellow solid identified as the tri-TFA salt of 3-(2-amino-pyrimidin-4-yl)-2-(6-methyl-pyridin-2-yl)-imidazo[1,2-a]pyridine-7-carboxylic acid (2-dimethylamino-ethyl)-amide. ¹H (d6-DMSO, 400 MHz, rotomers): 2.49 (s, 3H), 2.88 (s, 3H), 2.89 (s, 3H), 3.32 (t, J=5.8 Hz, 1H), 3.33 (t, J=5.8 Hz, 1H), 3.67 (dt, J=5.8 Hz, 1H), 6.80 (d, J=6.0 Hz, If, 7.41 (d, J=7.8 Hz, 1H), 7.52 (dd, J=7.3, 1.6 Hz, 1H), 7.82 (d, J=7.8 Hz, 1H), 7.98 (t, J=7.8 Hz, 1H), 8.23 (d, J=6.0 Hz, 1H), 8.34 (m, 1H), 9.08 (t, J=5.7 Hz, 1H), 9.46 (bs, 1H), 9.65 (d, J=7.3 Hz, 1H); MS (ESP+) 417.26 (M+1).

### EXAMPLE 94

### 3-(2-Amino-pyrimidin-4-yl)-2-(6-methyl-pyridin-2-yl)-imidazo[1,2-a]pyridine-7-carboxylic acid (2-oxo-2-pyridin-3-yl-ethyl)-amide

HATU (0.202 mmol), diisopropylethylamine (1.008 mmol) and 2-amino-1-pyridin-3-yl-ethanone hydrochloride (0.173 mmol) were added to a slurry of 3-(2-amino-pyrimidin-4-yl)-2-(6-methyl-pyridin-2-yl)-imidazo[1,2-a]pyridine-7-carboxylic acid (0.144 mmol; see Example 81 for its preparation) in N,N-dimethylformamide (1.4 mL) at RT. After 17.75 h, the reaction was concentrated in vacuo and purified via reverse phase HPLC (acetonitrile/water gradient with 0.1% TFA) to give 28 mg of a yellow solid identified as the tri-TFA salt of 3-(2-amino-pyrimidin-4-yl)-2-(6-methyl-pyridin-2-yl)-imidazo[1,2-a]pyridine-7-carboxylic acid (2-oxo-2-pyridin-3-yl-ethyl)-amide. ¹H (d6-DMSO, 400 MHz): 2.50 (s, 3H), 4.91 (d, J=5.3 Hz, 2H), 6.85 (d, J=6.1 Hz, 1H), 7.46 (d, J=7.8 Hz, 1H), 7.59 (dd, J=7.4, 1.7 Hz, 1H), 7.65 (dd, J=8.0, 4.9 Hz, 1H), 7.87 (m, 1H), 7.96 (m, 1H), 8.24 (d, J=6.1 Hz, 1H), 8.39 (m, 1H), 8.42 (m, if), 8.87 (dd, J=4.9, 1.7 Hz, 1H), 9.26 (m, 1H), 9.40 (t, J=5.8 Hz, 1H), 9.68 (d, J=7.4 Hz, 1H); MS (ESP+) 465.13 (M+1).

### EXAMPLE 95

### 3-(2-Amino-pyrimidin-4-yl)-2-(6-methyl-pyridin-2-yl)-imidazo[1,2-a]pyridine-7-carboxylic acid hydroxyamide

HATU (0.202 mmol), diisopropylethylamine (1.008 mmol) and hydroxylamine hydrochloride (0.173 mmol) were added to a slurry of 3-(2-amino-pyrimidin-4-yl)-2-(6-methyl-pyridin-2-yl)-imidazo[1,2-a]pyridine-7-carboxylic acid (0.144 mmol; see Example 81 for its preparation) in N,N-dimethylformamide (1.4 mL) at RT. After 22 h, the reaction was concentrated in vacuo and purified via reverse phase HPLC (acetonitrile/water gradient with 0.1% TFA) to give 27 mg of a yellow solid identified as the tri-TFA salt of 3-(2-amino-pyrimidin-4-yl)-2-(6-methyl-pyridin-2-yl)-imidazo[1,2-a]pyridine-7-carboxylic acid hydroxyamide. ¹H (d6-DMSO, 400 MHz): 2.69 (s, 3H), 6.81 (d, J=6.0 Hz, 1H), 7.43 (d, J=7.8 Hz, 1H), 7.46 (dd, J=7.3, 1.8 Hz, 1H), 7.83 (d, J=7.8 Hz, 1H), 7.94 (t, J=7.8 Hz, 1H), 8.17 (m, 1H), 8.22 (d, J=6.0 Hz, 1H), 9.62 (d, J=7.4 Hz, 1H), 11.64 (bs, 1H); MS (ESP+) 362.05 (M+1).

### EXAMPLE 96

### 2-(6-Methyl-pyridin-2-yl)-3-(2-methylsulfanyl-pyrimidin-4-yl)-imidazo[1,2-a]pyrimidin-7-ylamine

Hydrogen bromide/acetic acid (30 wt %, 75.0 mmol) and 1.00 M bromine/acetic acid (32.5 mmol) was added to a solution of 1-(6-methyl-pyridin-2-yl)-2-(2-methylsulfanyl-pyrimidin-4-yl)-ethanone (25.0 mmol; see Example 1(b) for its preparation) and catalytic BHT in glacial acetic acid (120 mL) at RT to form a precipitate. After 0.5 h the reaction was diluted to 400 mL with ether, filtered, washed with ether, dried briefly under N₂ gas to give an orange solid. The solid was added to a solution of 2,4-diaminopyrimidine (25.0 mmol) and diisopropylethylamine (100.0 mmol) in ethanol (270 mL) at 85° C. After 3.5 h the reaction was allowed to cool to 0° C. and precipitate was formed. The slurry was filtered, the solid washed with ethanol and air dried to give a solid. This solid was slurried with 2 N aqueous sodium hydroxide, filtered and washed with water to give 5.059 g of a solid identified as 2-(6-methyl-pyridin-2-yl)-3-(2-methylsulfanyl-pyrimidin-4-yl)-imidazo[1,2-a]pyrimidin-7-ylamine. ¹H NMR (d6-DMSO, 300 MHz): 2.41 (s, 3H), 2.56 (s, 3H), 6.45 (d, J=7.7, 1H), 7.24 (s, 2H), 7.25 (d, J=7.8 Hz, 1H), 7.38 (d, J=5.4 Hz, 1H), 7.73 (d, J=7.6 Hz, 1H), 7.80 (t, J=7.7 Hz, 1H), 8.45 (d, J=5.4 Hz, 1H), 9.10 (d, J=7.6 Hz, 1H); MS (ESP+) 350.4 (M+1).

### EXAMPLE 97

### N-[2-(6-Methyl-pyridin-2-yl)-3-(2-methylsulfanyl-pyrimidin-4-yl)-imidazo[1,2-a]pyrimidin-7-yl]-3-pyridin-3-yl-propionamide

N,N-Dimethylformamide (0.9 mmol) and thionyl chloride (54.0 mmol) were added to a slurry of 3-pyridinepropanoic acid (18.0 mmol) in chloroform (180 mL) at RT. The slurry was heated to 60° C. for 0.5 h, cooled to RT and concentrated in vacuo to yield a solid. This solid was added to a slurry of 2-(6-methyl-pyridin-2-yl)-3-(2-methylsulfanyl-pyrimidin-4-yl)-imidazo[1,2-a]pyrimidin-7-ylamine (1.14 mmol; see Example 96 for its preparation) in pyridine (40 mL) at 0° C. The slurry was warmed to RT. After 23.75 h, the slurry was warmed to 50° C. for a further 2.25 h. The reaction was cooled to RT, diluted with water (˜80 mL) and stirred for 0.5 h. The reaction was refrigerated at 0° C. After 3 d, the reaction was warmed to RT, filtered and washed with water to yield 398 mg of N-[2-(6-methyl-pyridin-2-yl)-3-(2-methylsulfanyl-pyrimidin-4-yl)-imidazo[1,2-a]pyrimidin-7-yl]-3-pyridin-3-yl-propionamide. ¹H NMR (d6-DMSO, 300 MHz): 2.41 (s, 3H), 2.58 (s, 3H), 2.5 (t, J=7.4 Hz, 2H), 2.96 (t, J=7.4 Hz, 2H), 7.32 (m, 2H), 7.42 (d, J=5.4 Hz, 1), 7.71 (d, J=7.8 Hz, 1H), 7.83 (m, 2H), 8.01 (d, J=7.8 Hz, 1H), 8.42 (dd, J=1.4, 4.8 Hz, 1H), 8.51 (m, 1H), 8.55 (d, J=5.4 Hz, 1H), 9.50 (d, J=7.8 Hz, 1H), 11.20 (s, 1H); MS (ESP+) 483.5 (M+1).

### EXAMPLE 98

### N-[3-(2-Methanesulfonyl-pyrimidin-4-yl)-2-(6-methyl-pyridin-2-yl)-imidazo[1,2-a]pyrimidin-7-yl]-3-pyridin-3-yl-propionamide

4.0N Sulfuric acid (0.2 mmol), catalytic sodium tungstate dihydrate, and 30 wt % hydrogen peroxide (2.71 mmol) were added to a slurry of N-[2-(6-methyl-pyridin-2-yl)-3-(2-methylsulfanyl-pyrimidin-4-yl)-imidazo[1,2-a]pyrimidin-7-yl]-3-pyridin-3-yl-propionamide (0.82 mmol; see Example 97 for its preparation) in methanol (20 mL) at 55° C. After 18.5 h, the reaction was diluted with water (20 mL) and warmed at 55° C. for an additional 0.75 h. The reaction was then cooled to RT, quenched with saturated sodium thiosulfate, neutralized with saturated sodium bicarbonate and then cooled to 0° C. for 1.5 h. The reaction was filtered, washed with 1:1 methanol/water to give 302 mg of N-[3-(2-methanesulfonyl-pyrimidin-4-yl)-2-(6-methyl-pyridin-2-yl)-imidazo[1,2-a]pyrimidin-7-yl]-3-pyridin-3-yl-propionamide. ¹H NMR (d6-DMSO, 300 MHz): 2.43 (s, 3H), 2.86 (t, J=7.4 Hz, 21), 2.96 (t, J=7.4 Hz, 2H), 3.51 (s, 3H), 7.33 (m, 2H), 7.70 (d, J=7.8 Hz, 1H), 7.89 (m, 2H), 8.04 (d, J=7.8 Hz, 1H), 8.14 (d, J=5.4 Hz, 1H), 8.41 (m, 1H), 8.51 (m, 1H), 8.97 (d, J=5.4 Hz, 1H), 9.56 (d, J=7.8 Hz, 1H), 11.27 (s, 1H); MS (ESP+) 515.6 (M+1).

### EXAMPLE 99

### N-[3-(2-Amino-pyrimidin-4-yl)-2-(6-methyl-pyridin-2-yl)-imidazo[1,2-a]pyrimidin-7-yl]-3-pyridin-3-yl-propionamide

A solution of N-[3-(2-methanesulfonyl-pyrimidin-4-yl)-2-(6-methyl-pyridin-2-yl)-imidazo[1,2-a]pyrimidin-7-yl]-3-pyridin-3-yl-propionamide (0.59 mmol; see Example 98 for its preparation) and ammonium hydroxide (17.70 mmol) in dioxane (10 mL) was warmed to 100° C. for 18 h. The reaction then concentrated in vacuo and diluted with water to give a precipitate that was filtered, washed with water and air dried. The resulting solid was purified via reverse phase HPLC (acetonitrile/water gradient with 0.1% TFA) to give 42 mg of a yellow solid identified as the tri-TFA salt of N-[3-(2-amino-pyrimidin-4-yl)-2-(6-methyl-pyridin-2-yl)-imidazo[1,2-a]pyrimidin-7-yl]-3-pyridin-3-yl-propionamide. ¹H NMR (d6-DMSO, 300 MHz): 2.52 (s, 3H), 2.94 (t, J=7.3 Hz, 2H), 3.10 (t, J=7.3 Hz, 2H), 6.85 (d, J=6.2 Hz, 1H), 7.43 (d, J=7.8 Hz, 1H), 7.78 (m, 2H), 7.93 (t, J=7.8 Hz, 1H), 8.03 (d, J=7.8 Hz, 1H), 8.16 (d, J=6.2 Hz, 1H), 8.26 (d, J=7.8 Hz, 1H), 8.68 (d, J=5.4 Hz, 1H), 8.77 (m, 1H), 10.08 (d, J=7.8 Hz, 1H), 11.38 (s, 1H); MS (ESP+) 452.19 (M+1).

### EXAMPLE 100

### N-[3-(2-Amino-pyrimidin-4-yl)-2-(6-methyl-pyridin-2-yl)-imidazo[1,2-a]pyrimidin-7-yl]-2-(3-methoxy-phenyl)-acetamide

3-Methoxyphenyl acetyl chloride (0.58 mmol) was added to a slurry of 2-(6-methyl-pyridin-2-yl)-3-(2-methylsulfanyl-pyrimidin-4-yl)-imidazo[1,2-a]pyrimidin-7-ylamine (0.29 mmol; see Example 96 for its preparation) in pyridine (3 mL) at RT and then warmed to 60° C. After 28 h, the reaction was cooled to RT and additional 2-methoxyphenyl acetyl chloride (1.16 mmol) was added. The reaction was then warmed to 80° C. for a further 21 hours. The reaction was cooled to RT, diluted with water (˜10 mL) and stirred for 0.25 h. The reaction then filtered and washed with water to yield 200 mg of an impure solid containing 2-(3-methoxy-phenyl)-N-[2-(6 methyl-pyridin-2-yl)-3-(2-methylsulfanyl-pyrimidin-4-yl)-imidazo[1,2-a]pyrimidin-7-yl]-acetamide. The crude material was used without further purification. MS (ESP+) 498.4 (M+1).

### EXAMPLE 101

### N-[3-(2-Methanesulfonyl-pyrimidin-4-yl)-2-(6-methyl-pyridin-2-yl)-imidazo[1,2-a]pyrimidin-7-yl]-2-(3-methoxy-phenyl)-acetamide

4.0N Sulfuric acid (0.1 mmol), catalytic sodium tungstate dihydrate, and 30 wt % hydrogen peroxide (1.32 mmol) were added to a slurry of 2-(3-methoxy-phenyl)-N-[2-(6-methyl-pyridin-2-yl)-3-(2-methylsulfanyl-pyrimidin-4-yl)-imidazo[1,2-a]pyrimidin-7-yl]-acetamide (0.40 mmol) in methanol (10 mL) and heated to 55° C. After 22.5 h, the reaction was diluted with water (10 mL) and warmed at 55° C. for an additional 1 h. The reaction was then cooled to RT, quenched with saturated sodium thiosulfate, neutralized with saturated sodium bicarbonate and then cooled to 0° C. overnight. The reaction warmed to RT, concentrated in vacuo and redissolved in ethyl acetate (25 mL) and water (10 mL). The organic and aqueous phases were separated and the organic layer was washed with water (1×10 mL), saturated sodium bicarbonate (1×10 mL), and brine (1×10 mL), dried (Na₂SO₄), and concentrated in vacuo to yield 180 mg of an impure solid containing N-[3-(2-methanesulfonyl-pyrimidin-4-yl)-2-(6-methyl-pyridin-2-yl)-imidazo[1,2-a]pyrimidin-7-yl]-2-(3-methoxy-phenyl)-acetamide. The crude material was used without further purification. MS (ESP+) 530.10 (M+1).

### EXAMPLE 102

### N-[3-(2-Amino-pyrimidin-4-yl)-2-(6-methyl-pyridin-2-yl)-imidazo[1,2-a]pyrimidin-7-yl]-2-(3-methoxy-phenyl)-acetamide

A solution of N-[3-(2-methanesulfonyl-pyrimidin-4-yl)-2-(6-methyl-pyridin-2-yl)-imidazo[1,2-a]pyrimidin-7-yl]-2-(3-methoxy-phenyl)-acetamide (0.34 mmol; see Example 101 for its preparation) and ammonium hydroxide (10.20 mmol) in dioxane (6 mL) was warmed to 100° C. for 18 h. The reaction concentrated in vacuo, diluted with water and stirred for 4 hours at RT. The aqueous layer was extracted with ethyl acetate (2×25 mL) The combined organic phases were washed with saturated sodium bicarbonate (1×20 mL) and brine (1×20 mL), dried (Na₂SO₄), and concentrated in vacuo. The resulting solid was purified via reverse phase HPLC (acetonitrile/water gradient with 0.1% TFA) to give 8 mg of a yellow solid identified as the bis-TFA salt of N-[3-(2-amino-pyrimidin-4-yl)-2-(6-methyl-pyridin-2-yl)-imidazo[1,2-a]pyrimidin-7-yl]-2-(3-methoxy-phenyl)-acetamide. ¹H NMR (d6-DMSO, 300 MHz): 2.53 (s, 3H), 3.76 (s, 3H), 3.81 (s, 2H), 6.83 (d, J=6.2 Hz, 1H), 6.86 (m, 1H), 6.94 (m, 2H), 7.26 (t, J=8.0 Hz, 1H), 7.43 (d, J=7.8 Hz, 1H), 7.77 (d, J=7.7 Hz, 1H), 7.93 (t, J=7.8 Hz, 1H), 8.04 (d, J=7.7 Hz, 1H), 8.14 (d, J=6.2 Hz, 1H), 10.07 (d, J=7.7 Hz, 1H), 11.53 (s, 1H); MS (ESP+) 467.20 (M+1).

### EXAMPLE 103

### N-[2-(6-Methyl-pyridin-2-yl)-3-(2-methylsulfanyl-pyrimidin-4-yl)-imidazo[1,2-a]pyrimidin-7-yl]-propionamide

Propionyl chloride (1.14 mmol) was added to a slurry of 2-(6-methyl-pyridin-2-yl)-3-(2-methylsulfanyl-pyrimidin-4-yl)-imidazo[1,2-a]pyrimidin-7-ylamine (0.57 mmol; see Example 96 for its preparation) in pyridine (5.7 mL) at RT and the slurry was warmed to 50° C. After 19 h, the reaction was cooled to RT and additional propionyl chloride (1.14 mmol) was added. The reaction was warmed to 50° C. for a further 5 h. The reaction was cooled to RT, diluted with water (˜15 mL) and refrigerated at 0° C. After 4 d, the reaction was warmed to RT, filtered and washed with water to yield 208 mg of N-[2-(6-methyl-pyridin-2-yl)-3-(2-methylsulfanyl-pyrimidin-4-yl)-imidazo[1,2-a]pyrimidin-7-yl]-propionamide. ¹H NMR (d6-DMSO, 300 MHz): 1.10 (t, 3H), 2.42 (s, 3H), 2.50 (q, 2H), 2.58 (s, 3H), 7.31 (dd, J=2.1, 6.5 Hz, 1H), 7.43 (d, J=5.4 Hz, 1H), 7.84 (m, 2H), 8.03 (d, J=7.7 Hz, 1H), 8.55 (d, J=5.4 Hz, 1H), 9.50 (d, J=7.7 Hz, 1H), 11.11 (s, 1H); MS (ESP+) 406.25 (M+1).

### EXAMPLE 104

### N-[3-(2-Methanesulfonyl-pyrimidin-4-yl)-2-(6-methyl-pyridin-2-yl)-imidazo[1,2-a]pyrimidin-7-yl]-propionamide

4.0N Sulfuric acid (0.1 mmol), catalytic sodium tungstate dihydrate, and 30 wt % hydrogen peroxide (1.69 mmol) were added to a slurry of N-[2-(6-methyl-pyridin-2-yl)-3-(2-methylsulfanyl-pyrimidin-4-yl)-imidazo[1,2-a]pyrimidin-7-yl]-propionamide (0.51 mmol; see Example 103 for its preparation) in methanol (13 mL) and heated to 55° C. After 19.5 h, the reaction was diluted with water (13 mL) and warmed at 55° C. for an additional 1 h. The reaction was then cooled to RT, quenched with saturated sodium thiosulfate, neutralized with saturated sodium bicarbonate and then cooled to 0° C. for 2 h. The reaction was filtered, washed with water to give 176 mg of N-[3-(2-methanesulfonyl-pyrimidin-4-yl)-2-(6-methyl-pyridin-2-yl)-imidazo[1,2-a]pyrimidin-7-yl]-propionamide. ¹H NMR (d6-DMSO, 300 MHz): 1.10 (t, J=7.5 Hz, 1H), 2.43 (s, 3H), 2.50 (q, 2H), 3.50 (s, 3H), 7.35 (dd, J=1.9, 6.7 Hz, 1H), 7.89 (m, 2H), 8.06 (d, J=7.8 Hz, 1H), 8.14 (d, J=5.4 Hz, 1H), 8.97 (d, J=5.4 Hz, 1H), 9.56 (d, J=7.8 Hz, 1H), 11.19 (s, M1); MS (ESP+) 438.4 (M+1).

### EXAMPLE 105

### N-[3-(2-Amino-pyrimidin-4-yl)-2-(6-methyl-pyridin-2-yl)-imidazo[1,2-a]pyrimidin-7-yl]-propionamide

A solution of N-[3-(2-methanesulfonyl-pyrimidin-4-yl)-2-(6-methyl-pyridin-2-yl)-imidazo[1,2-a]pyrimidin-7-yl]-propionamide (0.39 mmol; see Example 104 for its preparation) and ammonium hydroxide (11.73 mmol) in dioxane (9 mL) was warmed to 100° C. for 6 h. The reaction then concentrated in vacuo and the resulting solid was purified via reverse phase HPLC (acetonitrile/water gradient with 0.1% TFA) to give 120 mg of a yellow solid identified as the bis-TFA salt of N-[3-(2-amino-pyrimidin-4-yl)-2-(6-methyl-pyridin-2-yl)-imidazo[1,2-a]pyrimidin-7-yl]-propionamide. ¹H NMR (d6-DMSO, 300 MHz): 1.10 (t, J=7.5 Hz, 3H), 2.50 (q, 2H), 2.53 (s, 3H), 6.85 (d, J=6.2 Hz, 1H), 7.44 (d, J=7.8 Hz, 1H), 7.77 (d, J=7.8 Hz, 1H), 7.87 (bs, 2H), 7.94 (t, J=7.8 Hz, 1H), 8.10 (d, J=7.8 Hz, 1H), 8.15 (d, J=6.2 Hz, M1), 10.09 (d, J=7.8 Hz, 1H), 11.28 (s, 1H); MS (ESP+) 375.2 (M+1).

### EXAMPLE 106

### 3,3-Dimethyl-N-[2-(6-methyl-pyridin-2-yl)-3-(2-methylsulfanyl-pyrimidin-4-yl)-imidazo[1,2-a]pyrimidin-7-yl]-butyramide

Tert-butylacetyl chloride (0.58 mmol) was added to a slurry of 2-(6-methyl-pyridin-2-yl)-3-(2-methylsulfanyl-pyrimidin-4-yl)-imidazo[1,2-a]pyrimidin-7-ylamine (0.57 mmol; see Example 96 for its preparation) in pyridine (3 mL) at RT. After 25 h, additional tert-butylacetyl chloride (0.58 mmol) was added and the reaction was stirred for a further 17 h. The reaction diluted with ethyl acetate (20 mL) and water (10 mL). The organic and aqueous phases were separated and the organic layer was washed with water (1×10 mL) and brine (1×10 mL), dried (Na₂SO₄), and concentrated in vacuo to yield 173 mg of 3,3-dimethyl-N-[2-(6-methyl-pyridin-2-yl)-3-(2-methylsulfanyl-pyrimidin-4-yl)-imidazo[1,2-a]pyrimidin-7-yl]-butyramide. ¹H NMR (d6-DMSO, 300 MHz): 0.98 (s, 9H), 2.08 (s, 2H), 2.42 (s, 3H), 2.59 (s, 3H), 7.31 (dd, J=1.6, 6.6 Hz, 1H), 7.43 (d, J=5.4 Hz, 1H), 7.82 (m, 21), 8.06 (d, J=7.8 Hz, 1H), 8.55 (d, J=5.4 Hz, 1H), 9.51 (d, J=7.8 Hz, 1H), 11.06 (s, 1H); MS (ESP+) 448.5 (M+1).

### EXAMPLE 107

### N-[3-(2-Methanesulfonyl-pyrimidin-4-yl)-2-(6-methyl-pyridin-2-yl)-imidazo[1,2-a]pyrimidin-7-yl]-3,3-dimethyl-butyramide

4.0 N Sulfuric acid (0.1 mmol), catalytic sodium tungstate dihydrate, and 30 wt % hydrogen peroxide (1.25 mmol) were added to a slurry 3,3-dimethyl-N-[2-(6-methyl-pyridin-2-yl)-3-(2-methylsulfanyl-pyrimidin-4-yl)-imidazo[1,2-a]pyrimidin-7-yl]-butyramide (0.38 mmol; see Example 106 for its preparation) in methanol (10 mL) at 55° C. After 20.5 h, the reaction was diluted with water (10 mL) and warmed at 55° C. for an additional 0.75 h. The reaction was then cooled to RT, quenched with saturated sodium thiosulfate, quenched with saturated sodium bicarbonate and then cooled to 0° C. for 1.5 h. The reaction was filtered, washed with 1:1 methano/water to give 82 mg of N-[3-(2-methanesulfonyl-pyrimidin-4-yl)-2-(6-methyl-pyridin-2-yl)-imidazo[1,2-a]pyrimidin-7-yl]-3,3-dimethyl-butyramide. ¹H NMR (d6-DMSO, 300 MHz): 1.05 (s, 9H). 2.39 (s, 2H), 2.44 (s, 3H), 3.51 (s, 3H), 7.35 (dd, J=2.1, 6.5 Hz, 1H), 7.89 (m, 2H), 8.10 (d, J=7.8 Hz, 1H), 8.15 (d, J=5.4 Hz, 1H), 8.97 (d, J=5.4 Hz, 1H), 9.57 (d, J=7.8 Hz, 1H), 11.13 (s, 1H); MS (ESP+) 480.5 (M+1).

### EXAMPLE 108

### N-[3-(2-Amino-pyrimidin-4-yl)-2-(6-methyl-pyridin-2-yl)-imidazo[1,2-a]pyrimidin-7-yl]-3,3-dimethyl-butyramide

A solution of N-[3-(2-methanesulfonyl-pyrimidin-4-yl)-2-(6-methyl-pyridin-2-yl)-imidazo[1,2-a]pyrimidin-7-yl]-3,3-dimethyl-butyramide (0.17 mmol; see Example 107 for its preparation) and ammonium hydroxide (5.10 mmol) in dioxane (4 mL) was warmed to 100° C. for 19 h. The reaction concentrated in vacuo, diluted with water and stirred for 2 hours at RT. The resulting solid was filtered, washed with water, and purified via reverse phase HPLC (acetonitrile/water gradient with 0.1% TFA) to give 8 mg of a yellow solid identified as the bis-TFA salt of N-[3-(2-amino-pyrimidin-4-yl)-2-(6-methyl-pyridin-2-yl)-imidazo[1,2-a]pyrimidin-7-yl]-3,3-dimethyl-butyramide. ¹H NMR (d6-DMSO, 300 MHz): 1.05 (s, 9H), 2.39 (s, 2H), 2.53 (s, 3H), 6.85 (d, J=6.2 Hz, 1H), 7.43 (d, J=7.8 Hz, 1H), 7.67 (bs, 2H), 7.77 (d, J=7.8 Hz, 1H), 7.93 (t, J=7.8 Hz, 1H), 8.10 (d, J=7.8 Hz, 1H), 8.15 (d, J=6.2 Hz, 1H), 10.05 (d, J=7.8 Hz, 1H), 11.20 (s, 1H); MS (ESP+) 417.26 (M+1); MS (ESP−) 415.28 (M−1).

### EXAMPLE 109

### N-[2-(6-Methyl-pyridin-2-yl)-3-(2-methylsulfanyl-pyrimidin-4-yl)-imidazo[1,2-a]pyrimidin-7-yl]-nicotinamide

Nicotinoyl chloride hydrochloride (0.58 mmol) was added to a slurry of 2-(6-methyl-pyridin-2-yl)-3-(2-methylsulfanyl-pyrimidin-4-yl)-imidazo[1,2-a]pyrimidin-7-ylamine (0.29 mmol; see Example 96 for its preparation) in pyridine (3 mL) at RT. After 24 h, the reaction was diluted with water (10 mL), filtered and washed with water to yield 100 mg of N-[2-(6-methyl-pyridin-2-yl)-3-(2-methylsulfanyl-pyrimidin-4-yl)-imidazo[1,2-a]pyrimidin-7-yl]-nicotinamide. ¹H NMR (d6-DMSO, 300 MHz): 2.43 (s, 3H), 2.60 (s, 3H), 7.32 (m, 1H), 7.47 (d, J=5.3 Hz, 1H), 7.61 (dd, J=4.8, 8.0 Hz, 1H), 7.85 (m, 2H), 8.13 (d, J=7.7 Hz, 1H), 8.40 (dt, J=1.9, 8.0 Hz, 1H), 8.58 (d, J=5.3, 1H), 8.80 (dd, J=1.5, 4.8 Hz, 1H), 9.19 (m, 1H), 9.59 (d, J=7.7 Hz, 1H), 11.79 (s, 1H); MS (ESP+) 455.4 (M+1).

### EXAMPLE 110

### N-[3-(2-Methanesulfonyl-pyrimidin-4-yl)-2-(6-methyl-pyridin-2-yl)-imidazo[1,2-a]pyrimidin-7-yl]-nicotinamide

8289-042

4.0 N Sulfuric acid (0.04 mmol), catalytic sodium tungstate dihydrate, and 30 wt % hydrogen peroxide (0.63 mmol) were added to a slurry of N-[2-(6-methyl-pyridin-2-yl)-3-(2-methylsulfanyl-pyrimidin-4-yl)-imidazo[1,2-a]pyrimidin-7-yl]-nicotinamide (0.19 mmol; see Example 109 for its preparation) in methanol (5 mL) and warmed to 55° C. After 20.5 h, the reaction was diluted with water (5 mL) and warmed at 55° C. for an additional 0.75 h. The reaction was then cooled to RT, quenched with saturated sodium thiosulfate, neutralized with saturated sodium bicarbonate and then cooled to 0° C. for 1.5 h. The reaction was filtered, washed with 1:1 methanol/water to give 66 mg N-[3-(2-methanesulfonyl-pyrimidin-4-yl)-2-(6-methyl-pyridin-2-yl)-imidazo[1,2-a]pyrimidin-7-yl]-nicotinamide. ¹H NMR (d6-DMSO, 300 MHz): 2.44 (s, 3H), 3.53 (s, 3H), 7.36 (d, J=6.9 Hz, 1H), 7.60 (dd, J=4.7, 7.8 H z, 1H), 7.91 (m, 2H), 8.17 (m, 2H), 8.41 (d, J=7.8 Hz, 1H), 8.81 (d, J=4.6 Hz, 1H), 9.00 (d, J=5.3 Hz, 1H), 9.19 (m, 1H), 9.64 (d, J=7.8 Hz, 1H), 11.85 (s, 1H); MS (ESP+) 487.3 (M+1).

### EXAMPLE 111

### N-[3-(2-amino-pyrimidin-4-yl)-2-(6-methyl-pyridin-2-yl)-imidazo[1,2-a]pyrimidin-7-yl]-nicotinamide

A solution of N-[3-(2-methanesulfonyl-pyrimidin-4-yl)-2-(6-methyl-pyridin-2-yl)-imidazo[1,2-a]pyrimidin-7-yl]-nicotinamide (0.14 mmol; see Example 110 for its preparation) and ammonium hydroxide (4.20 mmol) in dioxane (3 mL) was warmed to 100° C. for 19 h. The reaction concentrated in vacuo, diluted with water and stirred for 2 hours at RT. The resulting solid was filtered, washed with water, and purified via reverse phase HPLC (acetonitrile/water gradient with 0.1% TFA) to give 8 mg of a yellow solid identified as the tri-TFA salt of N-[3-(2-amino-pyrimidin-4-yl)-2-(6-methyl-pyridin-2-yl)-imidazo[1,2-a]pyrimidin-7-yl]-nicotinamide. ¹H NMR (d₆-DMSO, 300 MHz): 2.53 (s, 3H), 6.88 (d, J=6.2 Hz, 1H), 7.43 (d, J=7.8 Hz, 1H), 7.61 (dd, J=4.9, 8.0 Hz, 1H), 7.80 (d, J=7.9 Hz, 1H), 7.94 (t, J=7.8 Hz, 1H), 8.16 (m, 2H), 8.42 (dt, J=2.0, 7.9 Hz, 1H), 8.82 (dd, J=1.7, 4.9 Hz, 1H), 9.20 (m, 1H), 10.15 (d, J=7.8 Hz, 1H), 11.90 (s, 1H); MS (ESP+) 424.27 (M+1); MS (ESP−) 422.28 (M−1).

### EXAMPLE 112

### 2-(6-Methyl-pyridin-2-yl)-3-pyrimidin-4-yl-imidazo[1,2-a]pyrimidin-7-ylamine

To a 100 mL flask was added 2-(6-methyl-pyridin-2-yl)-3-(2-methylsulfanyl-pyrimidin-4-yl)-imidazo[1,2-a]pyrimidin-7-ylamine (see Example 96 for its preparation), ethanol (18 mL) and ammonium hydroxide (15% aq., 16 mL). In another 100 mL flask, Raney nickel (slurry in water, ˜1.5 g) was washed several times, alternating between water and ethanol until the washes were no longer cloudy. After removing the last wash, the Raney nickel was transferred to the reaction flask using ethanol (˜18 mL) and the reaction was warmed to 78° C. After 21.5 h, the reaction was cooled to RT, filtered through Celite and rinsed with ethanol. The filtrate was concentrated in vacuo. The resulting material was purified via reverse phase HPLC (acetonitrile/water gradient with 0.1% TFA) to give 104 mg of a yellow solid identified as the TFA salt of 2-(6-methyl-pyridin-2-yl)-3-pyrimidin-4-yl-imidazo[1,2-a]pyrimidin-7-ylamine. ¹H NMR (d₆-DMSO, 300 MHz): 2.47 (s, 3H), 6.71 (d, J=7.7 Hz, 1H), 7.43 (d, J=7.6 Hz, 1H), 7.64 (d, J=7.8 Hz, M1), 7.68 (dd, J=1.1, 5.4 Hz, 1H), 7.89 (t, J=7.8 Hz, 1H), 8.84 (d, J=5.4 Hz, 1H), 9.08 (d, J=7.6 Hz, 1H), 9.35 (d, J=1.1 Hz, 1H); MS (ESP+) 304.22 (M+1).

Examples of compounds of the present invention are also described in the following table (Table I)

TABLE I

| Name | 1H NMR | MS m/z |
|---|---|---|
| [3-(2-amino-pyrimidin-4-yl)- | | 333.3 |
| 2-(6-methyl-pyridin-2-yl)- | | (M+1) |
| imidazo[1,2-a]pyridin-7-yl]- | | |
| methanol | | |
| [3-(4-Methyl-piperidin-1-yl)- | (d6-DMSO, 400MHz): 0.95(m, | 442.4 |
| propyl]-{4-[2-(6-methyl- | 3H), 1.35(m, 2H), 1.62(m, | (M+1) |
| pyridin-2-yl)-imidazo[1,2- | 1H), 1.81(m, 2H), 2.00(m, 2H), | |
| a]pyridin-3-yl]-pyrimidin- | 2.51(s, 3H), 2.93(m, 2H), | |
| 2-yl}-amine | 3.17(m, 2H), 3.47(m, 4H), | |
| | 6.76(bs, 1H), 7.20(t, 1H, | |
| | J=7.0Hz), 7.39(d, 1H, J=7.0Hz), | |
| | 7.62(m, 2H), 7.82(m, 2H), | |
| | 7.91(t, 1H, J=7.0Hz), 8.31(d, | |
| | 1H, J=4.7Hz), 9.17(bs, 1H) | |
| [4-(2-Pyridin-2-yl-imidazo[1, | | 380.2 |
| 2-a]pyridin-3-yl)-pyrimidin- | | (M+1) |
| 2-yl]-pyridin-3-ylmethyl- | | |
| amine | | |
| {4-[2-(6-methyl-pyridin-2- | | 343.2 |
| yl)-imidazo[1,2-a]-pyridin-3- | | (M+1) |
| yl]-pyrimidin-2-yl}-(1H- | | |
| tetrazol-5-yl)-amine | | |
| {4-[2-(6-Methyl-pyridin-2- | (d6-DMSO, 400MHz): 1.38(m, | 428.25 |
| yl)-imidazo[1,2-a]-pyridin- | 1H), 1.66(m, 3H), 1.80(m, 2H), | (M+1) |
| 3-yl]-pyrimidin-2-yl}-(3- | 2.49(s, 3H), 2.87(m, 2H), | |
| piperidin-1-yl-propyl)-amine | 3.13(m, 2H), 3.42(m, 4H), | |
| | 6.72(bs, 1H), 7.17(t, 1H, | |
| | J=7.3Hz), 7.37(d, 1H, J=7.3Hz), | |
| | 7.58(t, 1H, J=7.3Hz), 7.65(bs, | |
| | 1H), 7.83(m, 3H), 8.28(d, 1H, | |
| | J=5.3Hz), 9.32(bs, 1H) | |
| {7,7-Dimethyl-8-[5-(4-{4-[2- | | 859.1 |
| (6-methyl-pyridin-2-yl)- | | (M+1) |
| imidazo[1,2-a]pyridin-3-yl]- | | |
| pyrimidin-2-ylamino}-butyl- | | |
| carbamoyl)-pentyl]-2-oxo-4- | | |
| trifluoromethyl-7,8-dihydro- | | |
| 2H-1-oxa-8-aza-anthracen-5- | | |
| yl}-methanesulfonic acid | | |
| 2-(2,7-Difluoro-6-hydroxy-3- | | 868.4 |
| oxo-9,9a-dihydro-3H-xanthen- | | (M+1) |
| 9-yl)-3,5,6-trifluoro-4-[(4-{4- | | |
| [2-(6-methyl-pyridin-2-yl)- | | |
| imidazo[1,2-a]pyridin-3-yl]- | | |
| pyrimidin-2-ylamino}-butyl- | | |
| carbamoyl)-methylsulfanyl]- | | |
| benzoic acid | | |
| 2-(6-Methyl-pyridin-2-yl)-3- | | |
| pyrimidin-4-yl-imidazo[1,2- | | |
| a]pyridine | | |
| 3-(2-amino-pyrimidin-4-yl)-2- | (DMSO-d6, 400MHz)2.93(s, | 318.21 |
| (6-methyl-pyridin-2-yl)- | 3H), 6.63(d, J=7.48Hz, 1H), | (M+1) |
| imidazo[1,2-a]pyridin-8- | 6.66(d, J=7.72Hz, 1H), 7.89(dd, | |
| ylamine | J=7.77, 7.75Hz, 1H), 8.10(d, | |
| | J=6.12Hz, 1H), 8.89(d, | |
| | J=5.70Hz, 1H) | |
| 3-(2-amino-pyrimidin-4-yl)-2- | (DMSO-d6, 400MHz)2.42(s, | 328.2 |
| (6-methyl-pyridin-2-yl)- | 3H), 6.74(d, J=5.76Hz, 1H), | (M+1) |
| imidazo[1,2-a]pyridine-6- | 7.34(d, J=7.61Hz, 1H), 7.50(br | |
| carbonitrile | s, 2H), 7.74-7.77(m, 2H), | |
| | 7.85(dd, J=7.75, 7.71Hz, 1H), | |
| | 7.91(d, J=9.28Hz, 1H), 8.17(d, | |
| | J=5.75Hz, 1H), 10.08(s, 1H) | |
| 3-(2-Amino-pyrimidin-4-yl)- | | 486.24 |
| 2-(6-methyl-pyridin-2-yl)- | | (M+1) |
| imidazo[1,2-a]pyridine-6- | | |
| carboxylic acid [3-(4-ethyl- | | |
| piperazin-1-yl)-propyl]-amide | | |
| 3-(2-amino-pyrimidin-4-yl)-2- | (DMSO-d6, 400MHz)2.45(s, | 346.22 |
| (6-methyl-pyridin-2-yl)- | 3H), 6.81(d, J=5.69Hz, 1H), | (M+1) |
| imidazo[1,2-a]pyridine-6- | 7.34(d, J=7.49Hz, 1H), 7.44(br | |
| carboxylic acid amide | s, 2H), 7.64(br s, 1H), | |
| | 7.78-7.82(m, 2H), 7.84-7.89(m, | |
| | 2H), 8.12(br s, 1H), 8.23(d, | |
| | J=5.69Hz, 1H), 9.63(s, 1H) | |
| 3-(2-Amino-pyrimidin-4-yl)- | | 362.14 |
| 2-(6-methyl-pyridin-2-yl)- | | (M+1) |
| imidazo[1,2-a]pyridine-6- | | |
| carboxylic acid hydroxyamide | | |
| 3-(2-methanesulfonyl- | (DMSO-d6, 400MHz)2.34(s, | 391.18 |
| pyrimidin-4-yl)-2-(6-methyl- | 3H), 3.46(s, 3H), 7.30(d, | (M+1) |
| pyridin-2-yl)-imidazo[1,2- | J=7.61Hz, 1H), 7.78(dd, | |
| a]pyridine-6-carbonitrile | J=9.33Hz, 1.51Hz, 1H), | |
| | 7.84(dd, J=7.71, 7.75Hz, 1H), | |
| | 7.94(d, J=7.25Hz, 1H), 7.94(d, | |
| | J=9.61Hz, 1H), 8.16(d, | |
| | J=5.35Hz, 1H), 9.04(d, | |
| | J=5.35Hz, 1H), 9.80(s, 1H) | |
| 3-(2-Methylsulfanyl- | | 320.2 |
| pyrimidin-4-yl)-2-pyridin-2- | | (M+1) |
| yl-imidazo[1,2-a]pyridine | | |
| 3,6-Dichloro-N-(4-{4-[2-(6- | | 939.5 |
| methyl-pyridin-2-yl)- | | (M+1) |
| imidazo[1,2-a]pyridin-3-yl]- | | |
| pyrimidin-2-ylamino}-butyl)- | | |
| 2-(2,4,5,7-tetrachloro-6- | | |
| hydroxy-3-oxo-9,9a-dihydro- | | |
| 3H-xanthen-9-yl)-terephthala- | | |
| mic acid | | |
| 4-(2-Pyridin-2-yl-imidazo[1, | | 289.2 |
| 2-a]pyridin-3-yl)-pyrimidin-2- | | (M+1) |
| ylamine | | |
| 4-[2-(6-Chloro-pyridin-2-yl)- | | 323.1 |
| imidazo[1,2-a]pyridin-3-yl]- | | |
| pyrimidin-2-ylamine | | |
| 4-[2-(6-Methyl-pyridin-2-yl)- | | 371.2 |
| 7-trifluoromethyl-imidazo[1, | | (M+1) |
| 2-a]pyridin-3-yl]-pyrimidin-2- | | |
| ylamine | | |
| 4-[2-(6-methyl-pyridin-2-yl)- | | 313.2 |
| imidazo[1,2-a]pyridin-3-yl]- | | (M+1) |
| pyrimidine-2-carbonitrile | | |
| 4-[2-(6-methyl-pyridin-2-yl)- | | 331.2 |
| imidazo[1,2-a]pyridin-3-yl]- | | (M+1) |
| pyrimidine-2-carboxylic acid | | |
| amide | | |
| 4-[6-Bromo-2-(6-methyl- | (400MHz, CDCl₃)(J=Hz) | 381.1 |
| pyridin-2-yl)-imidazo[1,2- | δ 9.64(s; 1H), 8.15(d; 1H; | & |
| a]pyridin-3-yl]-pyrimidin-2- | J=6.0), 7.88(t; 1H; J=8.0); | 383.1 |
| ylamine | 7.76(d; 1H; J=9.4), 7.70(dd; | (M+1) |
| | 1H; J=9.7, 2.1), 7.36(d; 1H; | |
| | J=7.7), 6.77(d; 1H; J=6.3), | |
| | 2.43(s; 3H) | |
| 4-[6-Fluoro-2-(6-methyl- | (400MHz, CDCl₃)(J=Hz) | 321.2 |
| pyridin-2-yl)-imidazo[1,2- | δ 9.80(dd; 1H; J=3.6, 2.0), | (M+1) |
| a]pyridin-3-yl]-pyrimidin-2- | 8.11(d; 1H; J=6.3), 7.88(m; 2H); | |
| ylamine | 7.73(d; 1H; J=7.7), 7.69(dt; 1H; | |
| | J=7.9, 2.6), 7.37(d; 1H; J=8.0), | |
| | 6.75(d; 1H; J=6.2), 2.45(s, 3H). | |
| 4-[6-methyl-2-(6-methyl- | (DMSO-d6, 400MHz)2.39(s, | 317.2 |
| pyridin-2-yl)-imidazo[1,2- | 3H), 2.49(s, 3H), 6.73(d, | (M+1) |
| a]pyridin-3-yl]-pyrimidin- | J=5.99Hz, 1H), 7.40(d, | |
| 2-ylamine | J=7.69Hz, 1H), 7.55(dd, J=9.11, | |
| | 1.17Hz, 1H), 7.72(d, J=7.76, | |
| | 1H), 7.76(d, J=9.12Hz, 1H), | |
| | 7.89(dd, J=7.77, 7.77Hz, 1H), | |
| | 8.17(d, J=5.99Hz, 1H), | |
| | 9.37(s, 1H) | |
| 4-[7-aminomethyl-2-(6- | | 332.2 |
| methyl-pyridin-2-yl)- | | (M+1) |
| imidazo[1,2-a]pyridin-3-yl]- | | |
| pyrimidin-2-ylamine | | |
| 4-[8-Benzyloxy-2-(6-methyl- | | 410.1 |
| pyridin-2-yl)-imidazo[1,2- | | (M+1) |
| a]pyridin-3-yl]-pyrimidin-2-ol | | |
| 4-[8-Benzyloxy-2-(6-methyl- | (CDC₃, 400MHz)9.03(d, | 440.1 |
| pyridin-2-yl)-imidazo[1,2- | J=6.77Hz, 1H), 8.34(d, | (M+1) |
| a]pyridin-3-yl]-pyrimidin-2- | J=5.38Hz, 1H), 7.84(d, | |
| ylamine | J=7.73Hz, 1H), 7.68(dd, | |
| | J=7.73, 7.70Hz, 1H), 7.49(d, | |
| | J=7.16Hz, 2H), 7.36(dd, J=7.57, | |
| | 6.99Hz, 2H), 7.31(d, J=7.19Hz, | |
| | 1H), 7.16(d, J=5.43Hz, 2H), | |
| | 6.74(dd, J=7.44, 7.20Hz, 1H), | |
| | 5.41(s, 3H), 2.60(s, 3H), | |
| | 2.50(s, 3H) | |
| 5-Dimethylamino- | | 607.1 |
| naphthalene-1-sulfonic acid | | (M+1) |
| (4-{4-[2-(6-methyl-pyridin- | | |
| 2-yl)-imidazo[1,2-a]pyridin- | | |
| 3-yl]-pyrimidin-2-ylamino}- | | |
| butyl)-amide | | |
| 6-(2,7-Difluoro-6-hydroxy- | | 768.1 |
| 3-oxo-3H-xanthen-9-yl)-N- | | (M+1) |
| (4-{4-[2-(6-methyl-pyridin- | | |
| 2-yl)-imidazo[1,2-a]pyridin- | | |
| 3-yl]-pyrimidin-2-ylamino}- | | |
| butyl)-isothalamic acid | | |
| 6-Amino-9-[2-carboxy-5- | | 730.2 |
| (4-{4-[2-(6-methyl-pyridin- | | (M+1) |
| 2-yl)-imidazo[1,2-a]pyridin- | | |
| 3-yl]-pyrimidin-2-ylamino}- | | |
| butylcarbamoyl)-phenyl]- | | |
| xanthen-3-ylidene-ammonium | | |
| 6-Bromo-2-(6-methyl-pyridin- | (300MHz, CDCl₃)(J=Hz)δ | 412.1 |
| 2-yl)-3-(2-methylsulfanyl- | 9.73(s; 1H), 8.29(d; 1H; J=5.4), | & |
| pyrimidin-4-yl)-imidazo[1,2- | 7.62(m; 2H); 7.55(d; 1H; | 414.0 |
| a]pyridine | J=9.0); 7.36(dd; 1H; J=9.3, | (M+1) |
| | 1.8), 7.12(m; 2H), 2.60(s; 3H), | |
| | 2.48(s; 3H) | |
| 6-Fluoro-2-(6-methyl-pyridin- | (300MHz, CDCl₃)(J=Hz)δ | 352.1 |
| 2-yl)-3-(2-methylsulfanyl- | 9.92(s; 1H), 8.38(d; 1H; J=5.1), | (M+1) |
| pyrimidin-4-yl)-imidazo[1,2- | 7.76(m; 3H); 7.33(m; 3H); | |
| a]pyridine | 2.67(s; 3H), 2.57(s; 3H). | |
| 7-Amino-4-methyl-3-[(4-{4- | | 669.1 |
| [2-(6-methyl-pyridin-2-yl)- | | (M+1) |
| imidazo[1,2-a]pyridin-3- | | |
| yl]-pyrimidin-2-ylamino}- | | |
| butylcarbamoyl)-methyl]-2- | | |
| oxo-2H-chromene-6-sulfonic | | |
| acid | | |
| Cyclobutyl-{4-[2-(6- | (d6-DMSO, 400MHz): 1.72(m, | 329.3 |
| methyl-pyridin-2-yl)- | 2H), 2.07(m, 2H), 2.29(m, 2H), | (M+1) |
| imidazo[1,2-a]pyridin-3- | 2.55(s, 3H), 4.36(m, 1H), | |
| yl]-pyrimidin-2-yl}-amine | 6.72(d, 1H, J=3.5Hz, 7.30(t, | |
| | 1H, J=7.0Hz), 7.47(d, J=7.0Hz), | |
| | 7.70(t, 1H, J=7.0Hz), 7.78(d, | |
| | 1H, J=8.2Hz), 7.88(d, 1H, | |
| | J=9.4Hz), 7.97(t, 1H, J=9.4Hz), | |
| | 8.15(bs, 1H), 8.28(d, 1H, | |
| | J=4.7Hz), 9.41(bs, 1H) | |
| Cyclopropyl-methyl-{4-[2-(6- | (d6-DMSO, 400MHz): 0.78(m, | 351.3 |
| methyl-pyridin-2-yl)- | 2H), 0.89(m, 2H), 2.51(s, 3H), | (M+1) |
| imidazo[1,2-a]pyridin-3-yl]- | 2.88(m, 1H), 3.18(s, 3H), | |
| pyrimidin-2-yl}-amine | 6.81(d, 1H, J=5.7Hz), 7.26(t, | |
| | 1H, J=7.0Hz), 7.44(d, 1H, | |
| | J=7.5Hz), 7.65(m, 1H), 7.77(d, | |
| | 1H, J=7.8Hz), 7.85(d, 1H, | |
| | J=8.8Hz), 7.93(t, 1H, J=7.5Hz), | |
| | 8.37(d, 1H, J=5.7Hz), 9.60(d, | |
| | 1H, J=7.5Hz) | |
| Isopropyl-{4-[2-(6-methyl- | (d6-DMSO, 400MHz): 1.23(d, | 345.7 |
| pyridin-2-yl)-imidazo[1,2-a]- | 6H, J=6.8Hz), 2.55(s, 3H), | (M+1) |
| pyridin-3-yl]-pyrimidin-2-yl}- | 4.10(bs, 1H), 6.70(bs, 1H), | |
| amine | 7.28(t, 1H, J=6.8Hz), 7.46(d, | |
| | 1H, J=6.8Hz), 7.67(t, 1H, | |
| | J=8.1Hz), 7.78(d, 1H, J=8.1Hz), | |
| | 7.86(d, 1H, J=8.1Hz), 7.97(t, | |
| | 1H, J=9.6Hz), 8.26(d, 1H, | |
| | J=6.8Hz), 9.48(bs, 1H) | |
| N-(4-{4-[2-(6-Methyl- | | |
| pyridin-2-yl)-imidazo[1,2- | | |
| a]pyridin-3-yl]-pyrimidin-2- | | |
| ylamino}-butyl)- | | |
| (BODIPY-FL)amide | | |
| N-(4-{4-[2-(6-Methyl- | | 1075.1 |
| pyridin-2-yl)imidazo[1,2- | | (M+1) |
| a]pyridin-3-yl]-pyrimidin- | | |
| 2-ylamino}-butyl)- | | |
| (Texan Red-X) amide | | |
| N-[3-(2-amino-pyrimidin-4- | | 361.3 |
| yl)-2-(6-methyl-pyridin- | | (M+1) |
| 2-yl)-imidazo[1,2- | | |
| a]pyrimidin-7-yl]-acetamide | | |
| N-{4-[2-(6-methyl-pyridin-2- | (DMSO-d6, 400MHz)2.14(s, | 345.0 |
| yl)-imidazo[1,2-a]pyridin-3- | 3H), 2.44(s, 3H), 7.12(dd, | (M+1) |
| yl]-pyrimidin-2-yl}-acetamide | J=6.99, 6.92Hz, 1H), 7.18(d, | |
| | 5.46Hz, 1H), 7.36(d, J=7.66Hz, | |
| | 1H), 7.54(dd, J=8.09, 7.24Hz, | |
| | 1H), 7.75(d, J=6.15Hz, 1H), | |
| | 7.76(d, J=7.33Hz, 1H), 7.89(dd, | |
| | J=7.75, 7.69Hz, 1H) | |

Cell-Free Assay for Evaluating Inhibition of Activin Type I Receptor Kinase Activity

The TGFβ or activin inhibitory activity of compounds of formula (I) can be assessed by methods described in the following examples.

### EXAMPLE 113

Cell-Free Assay for Evaluating Inhibition of Autophosphorylation of TGFβ Type I Receptor

The serine-threonine kinase activity of TGFβ type I receptor was measured as the autophosphorylation activity of the cytoplasmic domain of the receptor containing an N-terminal poly histidine, TEV cleavage site-tag, e.g., His-TGFβRI. The His-tagged receptor cytoplasmic kinase domains were purified from infected insect cell cultures using the Gibco-BRL FastBac HTb baculovirus expression system.

To a 96-well Nickel FlashPlate (NEN Life Science, Perkin Elmer) was added 20 μl of 1.25 μCi ³³P-ATP/25 μM ATP in assay buffer (50 mM Hepes, 60 mM NaCl, 1 mM MgCl₂, 2 mM DTT, 5 nM MnCl₂, 2% glycerol, and 0.015% Brij 35). 10 μl of test compounds of formula (I) prepared in 5% DMSO solution were added to the FlashPlate. The assay was then initated with the addition of 20 ul of assay buffer containing 12.5 pmol of His-TGFβRI to each well. Plates were incubated for 30 minutes at room temperature and the reactions were then terminated by a single rinse with TBS. Radiation from each well of the plates was read on a TopCount (Packard). Total binding (no inhibition) was defined as counts measured in the presence of DMSO solution containing with no test compound and non-specific binding was defined as counts measured in the presence of EDTA or no-kinase control.

Alternatively, the reaction performed using the above reagents and incubation conditions but in a microcentrifuge tube was analyzed by separation on a 4-20% SDS-PAGE gel and the incorporation of radiolabel into the 40 kDa His-TGF□RI SDS-PAGE band was quantitated on a Storm Phosphoimager (Molecular Dynamics).

Compounds of formula (I) typically exhibited IC₅₀ values of less than 10 μM; some exhibited IC₅₀ values of less than 0.1 μM.

### EXAMPLE 114

Inhibition of the Activin type I receptor (Alk 4) kinase autophosphorylation activity by test compounds of formula (I) can be determined in a similar manner as described above in Example 7 except that a similarly His-tagged form of Alk 4 (His-Alk 4) was used in place of the His-TGFβRI.

### EXAMPLE 115

Assay for Evaluating Cellular Inhibition of TGFβ Signaling and Cytotoxicity

Biological activity of compounds of formula (I) were determined by measuring their ability to inhibit TGFβ-induced PAI-Luciferase reporter activity in HepG2 cells.

HepG2 cells were stably transfected with the PAI-luciferase reporter grown in DMEM medium containing 10% FBS, penicillin (100 U/ml), streptomycin (100 μg/ml), L-glutamine (2 mM), sodium pyruvate (1 mM), and non essential amino acids (1×). The transfected cells were then plated at a concentration of 2.5×10⁴ cells/well in 96 well plates and starved for 3-6 hours in media with 0.5% FBS at 37° C. in a 5% CO₂ incubator. The cells were then stimulated with ligand either 2.5 ng/ml TGFβ in the starvation media containing 1% DMSO and the presence or absence of test compounds of of formula (I) and incubated as described above for 24 hours. The media was washed out in the following day and the luciferase reporter activity was detected using the LucLite Luciferase Reporter Gene Assay kit (Packard, cat. no. 6016911) as recommended. The plates were read on a Wallac Microbeta plate reader, the reading of which was used to determine the IC₅₀ values of compounds of formula (I) for inhibiting TGFβ-induced PAI-Luciferase reporter activity in HepG2 cells. Compounds of formula (I) typically exhibited IC₅₀ values of less 10 uM.

Cytotoxicity was determined using the same cell culture conditions as described above. Specifically, cell viability was determined after overnight incubation with the CytoLite cell viability kit (Packard, cat. no. 6016901). Compounds of formula (I) typically exhibited LD₂₅ values greater than 10 μM.

### EXAMPLE 116

Assay for Evaluating Cellular Inhibition of TGFβ Signaling

The cellular inhibition of activin signaling activity by test compounds of formula (I) were determined in a similar manner as described above in Example 115 except that 100 ng/ml of activin is added to serum starved cells in place of the 2.5 ng/ml TGFβ.

### EXAMPLE 117

Assay for TGFβ-Induced Collagen Expression

Preparation of Immortalized Collagen Promotor-Green Fluorescent Protein Cells

Fibroblasts were derived from the skin of adult transgenic mice expressing Green Fluorescent Protein (GFP) under the control of the collagen 1A1 promoter (see Krempen, K. et al., Gene Exp. 8: 151-163 (1999)). Cells were immortalised with a temperature sensitive large T antigen that is active at 33° C. Cells are expanded at 33° C. then transferred to 37° C. so that the large T becomes inactive (see Xu, S. et al., Exp. Cell Res. 220: 407-414 (1995)). Over the course of about 4 days and one split, the cells cease proliferating. Cells are then frozen in aliquots sufficient for a single 96 well plate.

Assay of TGFβ-Induced Collagen-GFP Expression

Cells are thawed, plated in complete DMEM (contains nonessential amino acids, 1 mM sodium pyruvate and 2 mM L-glutamine) with 10% fetal calf serum and incubated overnight at 37° C., 5% CO₂. The following day, the cells are trypsinized and transferred into 96 well format with 30,000 cells per well in 50 μl complete DMEM containing 2% fetal calf serum, but without phenol red. The cells are incubated at 37° C. for 3 to 4 hours to allow them to adhere to the plate, solutions containing test compounds of formula (I) are then added to triplicate wells with no TGFβ, as well as triplicate wells with 1 ng/ml TGFβ. DMSO was also added to all of the wells at a final concentration of 0.1%. GFP fluorescence emission at 530 nm following excitation at 485 nm was measured at 48 hours after the addition of solution containing test compounds on a CytoFluor microplate reader (PerSeptive Biosystems). The data are then expressed as the ratio of TGFβ-induced to non-induced for each test sample.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound of the following formula: 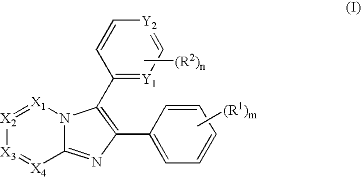
wherein
each of X₁, X₂, X₃, and X₄ is independently selected from CR^{x} or N; provided that only two of X₁, X₂, X₃, and X₄ can be N simultaneously;
each of Y₁ and Y₂ is independently selected from CR^{y} or N; provided that at least one of Y₁ and Y₂ must be N;
each R¹ is independently selected from alkyl, alkenyl, alkynyl, alkoxy, acyl, halo, hydroxy, amino, nitro, cyano, guanadino, amidino, carboxy, sulfo, mercapto, alkylsulfanyl, alkylsulfinyl, alkylsulfonyl, aminocarbonyl, alkylcarbonylamino, alkylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, heterocycloalkyl, heterocycloalkyloxy, heterocycloalkylsulfanyl, aryl, aryloxy, arylsulfanyl, aroyl, heteroaryl, heteroaryloxy, heteroarylsulfanyl, or heteroaroyl;
each R² is independently selected from alkyl, alkenyl, alkynyl, acyl, halo, hydroxy, —NH₂, —NH(alkyl), —N(alkyl)₂, —NH(cycloalkyl), —N(alkyl)(cyclocalkyl), —NH(heterocycloalkyl), —NH(heteroaryl), —NH-alkyl-heterocycloalkyl, —NH-alkyl-heteroaryl, —NH(aralkyl), cycloalkyl, (cycloalkyl)alkyl, aryl, aralkyl, aroyl, heterocycloalkyl, (heterocycloalkyl)alkyl, heteroaryl, heteroaralkyl, heteroaroyl, nitro, cyano, guanadino, amidino, carboxy, sulfo, mercapto, alkoxy, cycloalkyloxy, (cycloalkyl)alkoxy, aryloxy, arylalkoxy, heterocycloalkyloxy, (heterocycloalkyl)alkoxy, heteroaryloxy, heteroarylalkoxy, alkylsulfanyl, cycloalkylsulfanyl, (cycloalkyl)alkylsulfanyl, arylsulfanyl, aralkylsulfanyl, heterocycloalkylsulfanyl, (heterocycloalkyl)alkylsulfanyl, heteroarylsulfanyl, heteroarylalkylsulfanyl, alkylsulfinyl, alkylsulfonyl, aminocarbonyl, aminosulfonyl, alkylcarbonylamino, cycloalkylcarbonylamino, (cycloalkyl(alkylcarbonylamino, arylcarbonylamino, aralkylcarbonylamino, (heterocycloalkyl)carbonylamino, (heterocycloalkyl)alkylcarbonylamino, heteroarylcarbonylamino, heteroaralkylcarbonylamino, alkoxycarbonylaminoalkylamino, (heteroaryl)arylcarbonylaminoalkylamino, heteroaralkylcarbonylaminoalkylamino, (heteroaryl)arylsulfonylaminoalkylcarbonylaminoalkylamino, arylsulfonylaminoalkylamino, alkoxycarbonyl, alkylcarbonyloxy, urea, thiourea, sulfamoyl, sulfamide, or carbamoyl;
m is selected from 0, 1, 2, 3, or 4; provided that when m≧2, two adjacent R¹ groups can join together to form a 4- to 8-membered optionally substituted cyclic moiety;
n is selected from 0, 1, 2, or 3; provided that when n≧2, two adjacent R² groups can join together to form a 4- to 8-membered optionally substituted cyclic moiety; and
each of R^{x} and R^{y} is independently selected from hydrogen, alkyl, alkenyl, alkynyl, alkoxy, acyl, halo, hydroxy, amino, nitro, cyano, guanadino, amidino, carboxy, sulfo, mercapto, alkylsulfanyl, alkylsulfinyl, alkylsulfonyl, cycloalkylcarbonyl, (cycloalkyl)alkylcarbonyl, aroyl, aralkylcarbonyl, heterocycloalkylcarbonyl, (heterocycloalkyl)acyl, heteroaroyl, (heteroaryl)acyl, aminocarbonyl, alkylcarbonylamino, (amino)aminocarbonyl, alkylsulfonylaminocarbonyl, alkylsulfonylamino, cycloalkylcarbonylamino, cycloalkylsulfonylamino, (cycloalkyl)alkylcarbonylamino, (cycloalkyl)alkylsulfonylamino, arylcarbonylamino, arylsulfonylamino, aralkylcarbonylamino, aralkylsulfonylamino, (heterocycloalkyl)carbonylamino, (heterocycloalkyl)sulfonylamino, (heterocycloalkyl)alkylcarbonylamino, (heterocycloalkyl)alkylsulfonylamino, heteroarylcarbonylamino, heteroarylsulfonylamino, heteroaralkylcarbonylamino, heteroaralkylsulfonylamino, alkoxycarbonyl, alkylcarbonyloxy, urea, thiourea, sulfamoyl, sulfamide, carbamoyl, cycloalkyl, cycloalkyloxy, cycloalkylsulfanyl, (cycloalkyl)alkyl, (cycloalkyl)alkoxy, (cycloalkyl)alkylsulfanyl, heterocycloalkyl, heterocycloalkyloxy, heterocycloalkylsulfanyl, (heterocycloalkyl)alkyl, (heterocycloalkyl)alkoxy, (heterocycloalkyl)alkylsulfanyl, aryl, aryloxy, arylsulfanyl, aralkyl, aralkyloxy, aralkylsulfanyl, arylalkenyl, arylalkynyl, heteroaryl, heteroaryloxy, heteroarylsulfanyl, heteroaralkyl, (heteroaryl)alkoxy, or (heteroaryl)alkylsulfanyl;
or a pharmaceutically acceptable salt or N-oxide thereof.

2. The compound of claim 1, wherein each of X₁, X₂, and X₃ is CR^{x}.

3. The compound of claim 2, wherein each R^{x} is independently selected from hydrogen, unsubstituted alkyl, hydroxyalkyl, haloalkyl, aminoalkyl, aryloxyalkyl, heteroaralkyloxyalkyl, alkoxy, halo, hydroxy, carboxy, cyano, guanadino, amidino, amino, carboxy, (heteroaryl)acyl, alkoxycarbonyl, aminocarbonyl, alkylcarbonylamino, cycloalkylcarbonylamino, heteroarylcarbonylamino, (heterocycloalkyl)alkoxy, (heteroaryl)alkoxy, (heteroaryl)alkylsulfanyl, heterocycloalkyl, (heterocycloalkyl)alkyl, heteroaryl, or heteroaralkyl.

4. The compound of claim 2, wherein each R^{x} is independently selected from hydrogen, unsubstituted alkyl, hydroxyalkyl, trifluoromethyl, alkoxy, halo, hydroxy, cyano, guanadino, amidino, —NH₂, —NH(unsubstituted alkyl), —NH(hydroxyalkyl), —NH(alkoxyalkyl), —NH(carboxyalkyl), —N(unsubstituted alkyl)₂, —NH(heterocycloalkyl), —NH(heteroaryl), —NH(heterocycloalkylalkyl), —NH(aralkyl), —NH(heteroaralkyl), —NH—CO-alkyl, —NH—CO-heteroaryl, aminocarbonyl, heterocycloalkyl, or heteroaryl.

5. The compound of claim 2, wherein each R^{x} is hydrogen, methyl, ethyl, —NH₂, —NH—CO-methyl, —NH—CO-ethyl, —NH—CO-pyridyl, or —CO—NH(OH).

6. The compound of claim 2, wherein each of X₂, X₃, and X₄ is independently selected from —CH—, —C(CH₃)—, —C(OH)—, —C(NH₂)—, —C(CO—NH₂)—, —C(CO—NHOH)—, —C(NH(unsubstituted alkyl))-, —C(NH(aryl))-, —C(NH(aralkyl))-, —C(NH(heteroaryl))-, —C(NH(heteroarylalkyl))-, —C(NH—CO-(unsubstituted alkyl))-, —C(NH—CO-(aryl))-, —C(NH—CO-(heteroaryl))-, —C(NH—CO-(aralkyl))-, —C(NH—CO-(heteroarylalkyl))-, —C(NH—SO₂-(unsubstituted alkyl))-, —C(NH—SO₂-(aryl))-, —C(NH—SO₂-heteroaryl))-, —C(NH—SO₂-(aralkyl))-, —C(NH—SO₂-(heteroarylalkyl))-, —C(NH—SO₂—NH(unsubstituted alkyl))-, —C(NH—SO₂—NH(aryl))-, —C(NH—SO₂—NH(heteroaryl))-, —C(NH—SO₂—NH(aralkyl))-, —C(NH—SO₂—NH(heteroarylalkyl))-, —C(hydroxyalkyl)-, or —C(carboxy)-,
and X₁ is —CH—.

7. The compound of claim 2, wherein each of X₁ and X₂ is —CH—; X₄ is N; and X₃ is —C(NH₂)—, —C(NH(unsubstituted alkyl))-, —C(NH(aryl))-, —C(NH(aralkyl))-, —C(NH(heteroaryl))-, —C(NH(heteroarylalkyl))-, —C(NH—CO-(unsubstituted alkyl))-, —C(NH—CO-(aryl))-, —C(NH—CO-(heteroaryl))-, —C(NH—CO-(aralkyl))-, —C(NH—CO-(heteroarylalkyl))-, —C(NH—SO₂-(unsubstituted alkyl))-, —C(NH—SO₂—(aryl))-, —C(NH—SO₂-heteroaryl))-, —C(NH—SO₂-(aralkyl))-, —C(NH—SO₂-(heteroarylalkyl))-, —C(NH—SO₂—NH(unsubstituted alkyl))-, —C(NH—SO₂—NH(aryl))-, —C(NH—SO₂—NH(heteroaryl))-, —C(NH—SO₂—NH(aralkyl))-, or —C(NH—SO₂—NH(heteroarylalkyl))-.

8. The compound of claim 2, wherein m is selected from 0, 1, or 2.

9. The compound of claim 8, wherein each R¹ is independently selected from unsubstituted alkyl, hydroxyalkyl, haloalkyl, aminoalkyl, aryloxyalkyl, heteroaralkyloxyalkyl, unsubstituted alkenyl, alkoxy, acyl, halo, hydroxy, carboxy, cyano, guanadino, amidino, amino, carboxy, mercapto, alkylsulfanyl, alkylsulfinyl, alkylsulfonyl, aminocarbonyl, alkylcarbonylamino, alkoxycarbonyl, alkylcarbonyloxy, alkylsulfonyl, sulfamoyl, cycloalkyl, heterocycloalkyl, (heterocycloalkyl)alkyl, heteroaryl, or heteroaralkyl.

10. The compound of claim 8, wherein m is 1 and R¹ is selected from 6-alkyl, 6-alkenyl, 6-cycloalkyl, or 6-halo.

11. The compound of claim 8, wherein both Y₁ and Y₂ are N.

12. The compound of claim 11, wherein n is selected from 1 or 2 and each R² is independently selected from unsubstituted alkyl, hydroxyalkyl, haloalkyl, aminoalkyl, aryloxyalkyl, heteroaralkyloxyalkyl, alkoxy, acyl, halo, hydroxy, carboxy, cyano, guanadino, amidino, —NH₂, monoalkylamino, dialkylamino, monocycloalkylamino, monoheterocycloalkyl-amino, monoheteroaryl-amino, mono(heterocycloalkylalkyl)amino, mono (heteroaralkyl)amino, —N(alkyl)(cycloalkyl), mercapto, alkylsulfanyl, alkylsulfinyl, alkylsulfonyl, —CO—NH₂, —CO—NH(alkyl), —CO—N(alkyl)₂, —NH—CO-alkyl, —N(alkyl)-CO-alkyl, —CO₂-alkyl, —O—CO-alkyl, —SO₂—NH₂, —SO₂—NH(alkyl), —SO₂—N(alkyl)₂, —NH—SO₂-alkyl, —N(alkyl)-SO₂-alkyl, —NH—CO—NH(alkyl), —N(alkyl)-CO—NH(alkyl), —NH-alkyl-NH—CO-alkyl-heteroaryl, —NH-alkyl-NH—CO-aryl-heteroaryl, —NH-alkyl-NH—CO-alkyl-NH—SO₂-aryl-heteroaryl, —NH-alkyl-NH—SO₂-aryl, —NH—SO₂—NH(alkyl), —N(alkyl)-SO₂—NH(alkyl), heterocycloalkyl, or heteroaryl.

13. The compound of claim 12, wherein R² is substituted at the 3-position with a group selected from guanadino, amidino, —NH₂, monoalkylamino, dialkylamino, monocycloalkylamino, monoheterocycloalkylamino, monoheteroarylamino, mono((heterocycloalkyl)alkyl)amino, mono(heteroaralkyl)amino, —NH—CO—NH(alkyl), —N(alkyl)-CO—NH(alkyl), —NH-alkyl-NH—CO-alkyl-heteroaryl, —NH-alkyl-NH—CO-aryl-heteroaryl, —NH-alkyl-NH—CO-alkyl-NH—SO₂-aryl-heteroaryl, —NH-alkyl-NH—SO₂-aryl, —NH—SO₂—NH(alkyl), —N(alkyl)-SO₂—NH(alkyl), heterocycloalkyl, or heteroaryl.

14. The compound of claim 13, wherein m is 1 and R¹ is selected from 6-methyl, 6-ethyl, 6-propyl, 6-chloro, 6-trifluoromethyl, 6-vinyl, or 6-cyclopropyl.

15. The compound of claim 1, wherein m is selected from 0, 1, or 2.

16. The compound of claim 15, wherein R¹ is substituted at the 5-position or the 6-position.

17. The compound of claim 16, wherein R¹ is C₁₋₄ alkyl, C₁₋₄ alkoxy, C₁₋₄ alkylthio, halo, amino, aminocarbonyl, or alkoxycarbonyl.

18. The compound of claim 15, wherein each R¹ is independently selected from unsubstituted alkyl, hydroxyalkyl, haloalkyl, aminoalkyl, aryloxyalkyl, heteroaralkyloxyalkyl, unsubstituted alkenyl, alkoxy, acyl, halo, hydroxy, carboxy, cyano, guanadino, amidino, —NH₂, monoalkylamino, dialkylamino, monoheterocycloalkylamino, monoheteroarylamino, mono(heterocyclylalkyl)amino, mono(aralkyl)amino, mono(heteroaralkyl)amino, carboxy, mercapto, alkylsulfanyl, alkylsulfinyl, alkylsulfonyl, —CONH₂, —CONH(alkyl), —CO—N(alkyl)₂, —NH—CO-alkyl, —N(alkyl)-CO-alkyl, —CO₂-alkyl, —O—CO-alkyl, —SO₂—NH₂, —SO₂—NH(alkyl), —SO₂—N(alkyl)₂, cycloalkyl, heterocycloalkyl, or heteroaryl.

19. The compound of claim 18, wherein m is 1 and R¹ is selected from 6-methyl, 6-ethyl, 6-propyl, 6-chloro, 6-trifluoromethyl, 6-ethyl, 6-vinyl, or 6-cyclopropyl.

20. The compound of claim 1, wherein both Y₁ and Y₂ are N.

21. The compound of claim 20, wherein n is selected from 1 or 2 and each R² is independently selected from unsubstituted alkyl, hydroxyalkyl, haloalkyl, aminoalkyl, aryloxyalkyl, heteroaralkyloxyalkyl, alkoxy, acyl, halo, hydroxy, carboxy, cyano, guanadino, amidino, —NH₂, monoalkylamino, dialkylamino, monocycloalkylamino, monoheterocycloalkylamino, monoheteroaryl-amino, mono(heterocycloalkylalkyl)-amino, mono(heteroaralkyl)amino, —N(alkyl)(cycloalkyl), mercapto, alkylsulfanyl, alkylsulfinyl, alkylsulfonyl, —CONH₂, —CONH(alkyl), —CO—N(alkyl)₂, —NH—CO-alkyl, —N(alkyl)-CO-alkyl, —CO₂-alkyl, —O—CO-alkyl, —SO₂—NH₂, —SO₂—NH(alkyl), —SO₂—N(alkyl)₂, —NH—SO₂-alkyl, —N(alkyl)-SO₂-alkyl, —NH—CO—NH(alkyl), —N(alkyl)-CO—NH(alkyl), —NH—SO₂—NH(alkyl), —N(alkyl)-SO₂—NH(alkyl), —NH-alkyl-NH—CO-alkyl-heteroaryl, —NH-alkyl-NH—CO-aryl-heteroaryl, —NH-alkyl-NH—CO-alkyl-NH—SO₂-aryl-heteroaryl, —NH-alkyl-NH—SO₂-aryl, heterocycloalkyl, or heteroaryl.

22. The compound of claim 21, wherein n is 1 and each R² is independently selected from guanadino, amidino, —NH₂, monoalkylamino, dialkylamino, monocycloalkylamino, monoheterocycloalkylamino, monoheteroarylamino, mono((heterocycloalkyl)alkyl)amino, mono(heteroaralkyl)amino, —NH—CO—NH(alkyl), —N(alkyl)-CO—NH(alkyl), —NH—SO₂—NH(alkyl), —N(alkyl)-SO₂—NH(alkyl), —NH-alkyl-NH—CO-alkyl-heteroaryl, —NH-alkyl-NH—CO-aryl-heteroaryl, —NH-alkyl-NH—CO-alkyl-NH—SO₂-aryl-heteroaryl, —NH-alkyl-NH—SO₂-aryl, heterocycloalkyl, or heteroaryl.

23. The compound of claim 22, wherein R² is substituted at the 3-position.

24. The compound of claim 1, wherein each of X₂, X₃, and X₄ is independently selected from —CH—, —C(OH)—, —C(NH₂)—, —C(NH(unsubstituted alkyl))-, —C(NH(aryl))-, —C(NH(aralkyl))-, —C(NH(heteroaryl))-, —C(NH(heteroarylalkyl))-, —C(NH—CO-(unsubstituted alkyl))-, —C(NH—CO-(aryl))-, —C(NH—CO-(heteroaryl))-, —C(NH—CO-(aralkyl))-, —C(NH—CO-(heteroarylalkyl))-, —C(NH—SO₂-(unsubstituted alkyl))-, —C(NH—SO₂-(aryl))-, —C(NH—SO₂-(heteroaryl))-, —C(NH—SO₂-(aralkyl))-, —C(NH—SO₂-(heteroarylalkyl))-, —C(NH—SO₂—NH(unsubstituted alkyl))-, —C(NH—SO₂—NH(aryl))-, —C(NH—SO₂—NH(heteroaryl))-, —C(NH—SO₂—NH(aralkyl))-, —C(NH—SO₂—NH(heteroarylalkyl))-, —C(hydroxyalkyl)-, —C(carboxy)-, or N.

25. The compound of claim 1, wherein X₁ is —CH—.

26. The compound of claim 1, wherein X₁ is N.

27. The compound of claim 1, wherein X₂ is N.

28. The compound of claim 1, wherein X₃ is N.

29. The compound of claim 1, wherein X₄ is N.

30. The compound of claim 1, selected from
(2-Methoxy-ethyl)-{4-[2-(6-methyl-pyridin-2-yl)-imidazo[1,2-a]pyridin-3-yl]-pyrimidin-2-yl}-amine;
(3-{4-[2-(6-Methyl-pyridin-2-yl)-imidazo[1,2-a]pyridin-3-yl]-pyrimidin-2-ylamino}-propyl)-carbamic acid tert-butyl ester;
(3-Imidazol-1-yl-propyl)-{4-[2-(6-methyl-pyridin-2-yl)-imidazo[1,2-a]pyridin-3-yl]-pyrimidin-2-yl}-amine;
(4-Methoxy-benzyl)-{4-[2-(6-methyl-pyridin-2-yl)-imidazo[1,2-a]pyridin-3-yl]-pyrimidin-2-yl}-amine;
[2-(6-Methyl-pyridin-2-yl)-3-(2-methylsulfanyl-pyrimidin-4-yl)-imidazo[1,2-a]pyridin-6-yl]-methanol;
3-(2-Methanesulfonyl-pyrimidin-4-yl)-2-(6-methyl-pyridin-2-yl)-imidazo[1,2-a]pyridine;
(4-{4-[2-(6-Methyl-pyridin-2-yl)-imidazo[1,2-a]pyridin-3-yl]-pyrimidin-2-ylamino}-butyl)-carbamic acid tert-butyl ester;
(4-Amino-benzyl)-{4-[2-(6-methyl-pyridin-2-yl)-imidazo[1,2-a]pyridin-3-yl]-pyrimidin-2-yl}-amine;
(5-{4-[2-(6-Methyl-pyridin-2-yl)-imidazo[1,2-a]pyridin-3-yl]-pyrimidin-2-ylamino}-pentyl)-carbamic acid tert-butyl ester;
[3-(2-Amino-pyrimidin-4-yl)-2-(6-methyl-pyridin-2-yl)-imidazo[1,2-a]pyridin-6-yl]-methanol;
[3-(2-amino-pyrimidin-4-yl)-2-(6-methyl-pyridin-2-yl)-imidazo[1,2-a]pyridin-7-yl]-methanol;
[3-(2-Amino-pyrimidin-4-yl)-6-methyl-2-(6-methyl-pyridin-2-yl)-imidazo[1,2-a]pyridin-8-yl]-(2-morpholin-4-yl-ethyl)-amine;
[3-(2-Amino-pyrimidin-4-yl)-6-methyl-2-(6-methyl-pyridin-2-yl)-imidazo[1,2-a]pyridin-8-yl]-(2-pyridin-2-yl-ethyl)-amine;
[3-(2-Amino-pyrimidin-4-yl)-6-methyl-2-(6-methyl-pyridin-2-yl)-imidazo[1,2-a]pyridin-8-yl]-(2-pyridin-3-yl-ethyl)-amine;
[3-(2-methanesulfonyl-pyrimidin-4-yl)-2-(6-methyl-pyridin-2-yl)-imidazo[1,2-a]pyridin-6-yl]-methanol;
[3-(2-Amino-pyrimidin-4-yl)-6-methyl-2-(6-methyl-pyridin-2-yl)-imidazo[1,2-a]pyridin-8-yl]-(2-pyridin-4-yl-ethyl)-amine;
[3-(2-Amino-pyrimidin-4-yl)-6-methyl-2-(6-methyl-pyridin-2-yl)-imidazo[1,2-a]pyridin-8-yl]-(3-morpholin-4-yl-propyl)-amine;
[3-(4-Methyl-piperazin-1-yl)-propyl]-{4-[2-(6-methyl-pyridin-2-yl)-imidazo[1,2-a]pyridin-3-yl]-pyrimidin-2-yl}-amine;
[3-(4-Methyl-piperidin-1-yl)-propyl]-{4-[2-(6-methyl-pyridin-2-yl)-imidazo[1,2-a]pyridin-3-yl]-pyrimidin-2-yl}-amine;
[4-(2-Pyridin-2-yl-imidazo[1,2-a]pyridin-3-yl)-pyrimidin-2-yl]-pyridin-3-ylmethyl-amine;
{4-[2-(6-Methyl-pyridin-2-yl)-imidazo[1,2-a]pyridin-3-yl]-pyrimidin-2-yl}-((R)-1-phenyl-ethyl)-amine;
{4-[2-(6-Methyl-pyridin-2-yl)-imidazo[1,2-a]pyridin-3-yl]-pyrimidin-2-yl}-((S)-1-phenyl-ethyl)-amine;
{4-[2-(6-Methyl-pyridin-2-yl)-imidazo[1,2-a]pyridin-3-yl]-pyrimidin-2-yl}-(1H-tetrazol-5-yl)-amine;
{4-[2-(6-Methyl-pyridin-2-yl)-imidazo[1,2-a]pyridin-3-yl]-pyrimidin-2-yl}-(2H-pyrazol-3-yl)-amine;
{4-[2-(6-Methyl-pyridin-2-yl)-imidazo[1,2-a]pyridin-3-yl]-pyrimidin-2-yl}-(2-morpholin-4-yl-ethyl)-amine;
{4-[2-(6-Methyl-pyridin-2-yl)-imidazo[1,2-a]pyridin-3-yl]-pyrimidin-2-yl}-(2-pyridin-2-yl-ethyl)-amine;
{4-[2-(6-Methyl-pyridin-2-yl)-imidazo[1,2-a]pyridin-3-yl]-pyrimidin-2-yl}-(2-pyridin-3-yl-ethyl)-amine;
{4-[2-(6-Methyl-pyridin-2-yl)-imidazo[1,2-a]pyridin-3-yl]-pyrimidin-2-yl}-(2-pyridin-4-yl-ethyl)-amine;
{4-[2-(6-Methyl-pyridin-2-yl)-imidazo[1,2-a]pyridin-3-yl]-pyrimidin-2-yl}-(3-morpholin-4-yl-propyl)-amine;
{4-[2-(6-Methyl-pyridin-2-yl)-imidazo[1,2-a]pyridin-3-yl]-pyrimidin-2-yl}-(3-piperidin-1-yl-propyl)-amine;
{4-[2-(6-Methyl-pyridin-2-yl)-imidazo[1,2-a]pyridin-3-yl]-pyrimidin-2-yl}-[1,3,4]thiadiazol-2-yl-amine;
2-(6-Methyl-pyridin-2-yl)-3-(2-methylsulfanyl-pyrimidin-4-yl)-imidazo[1,2-a]pyridine;
2-(6-Methyl-pyridin-2-yl)-3-(2-methylsulfanyl-pyrimidin-4-yl)-imidazo[1,2-a]pyridine-6-carboxylic acid methyl ester;
2-(6-Methyl-pyridin-2-yl)-3-(2-methylsulfanyl-pyrimidin-4-yl)-imidazo[1,2-a]pyridine-7-carboxylic acid ethyl ester;
2-(6-Methyl-pyridin-2-yl)-3-(2-methylsulfanyl-pyrimidin-4-yl)-imidazo[1,2-a]pyrimidin-7-ylamine;
{7,7-Dimethyl-8-[5-(4-{4-[2-(6-methyl-pyridin-2-yl)-imidazo[1,2-a]pyridin-3-yl]-pyrimidin-2-ylamino}-butylcarbamoyl)-pentyl]-2-oxo-4-trifluoromethyl-7,8-dihydro-2H-1-oxa-8-aza-anthracen-5-yl}-methanesulfonic acid;
2-(2,7-Difluoro-6-hydroxy-3-oxo-9,9a-dihydro-3H-xanthen-9-yl)-3,5,6-trifluoro-4-[(4-{4-[2-(6-methyl-pyridin-2-yl)-imidazo[1,2-a]pyridin-3-yl]-pyrimidin-2-ylamino}-butylcarbamoyl)-methylsulfanyl]-benzoic acid;
2-(6-Methyl-pyridin-2-yl)-3-(2-morpholin-4-yl-pyrimidin-4-yl)-imidazo[1,2-a]pyridine;
2-(6-Methyl-pyridin-2-yl)-3-(2-piperidin-1-yl-pyrimidin-4-yl)-imidazo[1,2-a]pyridine;
2-(6-Methyl-pyridin-2-yl)-3-(2-pyrrolidin-1-yl-pyrimidin-4-yl)-imidazo[1,2-a]pyridine;
2-(6-Methyl-pyridin-2-yl)-3-[2-(1H-tetrazol-5-yl)-pyrimidin-4-yl]-imidazo[1,2-a]pyridine;
2-(6-Methyl-pyridin-2-yl)-3-pyrimidin-4-yl-imidazo[1,2-a]pyridine;
2-(6-Methyl-pyridin-2-yl)-3-pyrimidin-4-yl-imidazo[1,2-a]pyrimidin-7-ylamine;
3-(2-Amino-pyrimidin-4-yl)-2-(6-methyl-pyridin-2-yl)-imidazo[1,2-a]pyridin-8-ylamine;
3-(2-Amino-pyrimidin-4-yl)-2-(6-methyl-pyridin-2-yl)-imidazo[1,2-a]pyridine-6-carbonitrile;
3-(2-Amino-pyrimidin-4-yl)-2-(6-methyl-pyridin-2-yl)-imidazo[1,2-a]pyridine-6-carboxylic acid;
3-(2-Amino-pyrimidin-4-yl)-2-(6-methyl-pyridin-2-yl)-imidazo[1,2-a]pyridine-6-carboxylic acid ([1,4]dioxan-2-ylmethyl)-amide;
3-(2-Amino-pyrimidin-4-yl)-2-(6-methyl-pyridin-2-yl)-imidazo[1,2-a]pyridine-6-carboxylic acid ([1,4]dioxan-2-ylmethyl)-amide;
3-(2-Amino-pyrimidin-4-yl)-2-(6-methyl-pyridin-2-yl)-imidazo[1,2-a]pyridine-6-carboxylic acid (2-dimethylamino-ethyl)-amide;
3-(2-Amino-pyrimidin-4-yl)-2-(6-methyl-pyridin-2-yl)-imidazo[1,2-a]pyridine-6-carboxylic acid (2-methoxy-ethyl)-amide;
3-(2-Amino-pyrimidin-4-yl)-2-(6-methyl-pyridin-2-yl)-imidazo[1,2-a]pyridine-6-carboxylic acid (2-thiophen-2-yl-ethyl)-amide;
3-(2-Amino-pyrimidin-4-yl)-2-(6-methyl-pyridin-2-yl)-imidazo[1,2-a]pyridine-6-carboxylic acid [3-(4-methyl-piperazin-1-yl)-propyl]-amide;
3-(2-Amino-pyrimidin-4-yl)-2-(6-methyl-pyridin-2-yl)-imidazo[1,2-a]pyridine-6-carboxylic acid amide;
3-(2-Amino-pyrimidin-4-yl)-2-(6-methyl-pyridin-2-yl)-imidazo[1,2-a]pyridine-6-carboxylic acid cyclopropylamide;
3-(2-Amino-pyrimidin-4-yl)-2-(6-methyl-pyridin-2-yl)-imidazo[1,2-a]pyridine-6-carboxylic acid ethylamide;
3-(2-Amino-pyrimidin-4-yl)-2-(6-methyl-pyridin-2-yl)-imidazo[1,2-a]pyridine-6-carboxylic acid hydroxyamide;
3-(2-Amino-pyrimidin-4-yl)-2-(6-methyl-pyridin-2-yl)-imidazo[1,2-a]pyridine-6-carboxylic acid methoxy-amide;
3-(2-Amino-pyrimidin-4-yl)-2-(6-methyl-pyridin-2-yl)-imidazo[1,2-a]pyridine-6-carboxylic acid methyl ester;
3-(2-Amino-pyrimidin-4-yl)-2-(6-methyl-pyridin-2-yl)-imidazo[1,2-a]pyridine-7-carboxylic acid;
3-(2-Amino-pyrimidin-4-yl)-2-(6-methyl-pyridin-2-yl)-imidazo[1,2-a]pyridine-7-carboxylic acid ([1,4]dioxan-2-ylmethyl)-amide;
3-(2-Amino-pyrimidin-4-yl)-2-(6-methyl-pyridin-2-yl)-imidazo[1,2-a]pyridine-7-carboxylic acid (2-amino-ethyl)-amide;
3-(2-Amino-pyrimidin-4-yl)-2-(6-methyl-pyridin-2-yl)-imidazo[1,2-a]pyridine-7-carboxylic acid (2-dimethylamino-ethyl)-amide;
3-(2-Amino-pyrimidin-4-yl)-2-(6-methyl-pyridin-2-yl)-imidazo[1,2-a]pyridine-7-carboxylic acid (2-hydroxy-ethyl)-amide;
3-(2-Amino-pyrimidin-4-yl)-2-(6-methyl-pyridin-2-yl)-imidazo[1,2-a]pyridine-7-carboxylic acid (2-oxo-2-pyridin-3-yl-ethyl)-amide;
3-(2-Amino-pyrimidin-4-yl)-2-(6-methyl-pyridin-2-yl)-imidazo[1,2-a]pyridine-7-carboxylic acid (2-thiophen-2-yl-ethyl)-amide;
3-(2-Amino-pyrimidin-4-yl)-2-(6-methyl-pyridin-2-yl)-imidazo[1,2-a]pyridine-7-carboxylic acid (piperidin-3-ylmethyl)-amide;
3-(2-Amino-pyrimidin-4-yl)-2-(6-methyl-pyridin-2-yl)-imidazo[1,2-a]pyridine-7-carboxylic acid 2,2-dimethylhydrazide;
3-(2-Amino-pyrimidin-4-yl)-2-(6-methyl-pyridin-2-yl)-imidazo[1,2-a]pyridine-7-carboxylic acid amide;
3-(2-Amino-pyrimidin-4-yl)-2-(6-methyl-pyridin-2-yl)-imidazo[1,2-a]pyridine-7-carboxylic acid cyclopropylamide;
3-(2-Amino-pyrimidin-4-yl)-2-(6-methyl-pyridin-2-yl)-imidazo[1,2-a]pyridine-7-carboxylic acid ethyl ester;
3-(2-Amino-pyrimidin-4-yl)-2-(6-methyl-pyridin-2-yl)-imidazo[1,2-a]pyridine-7-carboxylic acid ethylamide;
3-(2-Amino-pyrimidin-4-yl)-2-(6-methyl-pyridin-2-yl)-imidazo[1,2-a]pyridine-7-carboxylic acid hydroxyamide;
3-(2-Amino-pyrimidin-4-yl)-2-(6-methyl-pyridin-2-yl)-imidazo[1,2-a]pyridine-7-carboxylic acid methoxy-amide;
3-(2-Amino-pyrimidin-4-yl)-2-(6-methyl-pyridin-2-yl)-imidazo[1,2-a]pyrimidin-7-ylamine;
3-(2-Azetidin-1-yl-pyrimidin-4-yl)-2-(6-methyl-pyridin-2-yl)-imidazo[1,2-a]pyridine;
3-(2-Methanesulfonyl-pyrimidin-4-yl)-2-(6-methyl-pyridin-2-yl)-imidazo[1,2-a]pyridine-7-carboxylic acid ethyl ester;
3-(2-Methanesulfonyl-pyrimidin-4-yl)-2-(6-methyl-pyridin-2-yl)-imidazo[1,2-a]pyridine-6-carboxylic acid methyl ester;
3-(2-Methanesulfonyl-pyrimidin-4-yl)-7-methyl-2-(6-methyl-pyridin-2-yl)-imidazo[1,2-a]pyridine;
3-(2-Methanesulfonyl-pyrimidin-4-yl)-8-methyl-2-(6-methyl-pyridin-2-yl)-imidazo[1,2-a]pyridine;
3,3-Dimethyl-N-[2-(6-methyl-pyridin-2-yl)-3-(2-methylsulfanyl-pyrimidin-4-yl)-imidazo[1,2-a]pyrimidin-7-yl]-butyramide;
3-(2-Methanesulfonyl-pyrimidin-4-yl)-2-(6-methyl-pyridin-2-yl)-imidazo[1,2-a]pyridine-6-carbonitrile;
3-(2-Methylsulfanyl-pyrimidin-4-yl)-2-pyridin-2-yl-imidazo[1,2-a]pyridine;
3,6-Dichloro-N-(4-{4-[2-(6-methyl-pyridin-2-yl)-imidazo[1,2-a]pyridin-3-yl]-pyrimidin-2-ylamino}-butyl)-2-(2,4,5,7-Tetrachloro-6-hydroxy-3-oxo-9,9a-dihydro-3H-xanthen-9-yl)-terephthalamic acid;
3-[2-(2-Methyl-aziridin-1-yl)-pyrimidin-4-yl]-2-(6-methyl-pyridin-2-yl)-imidazo[1,2-a]pyridine;
3-[2-(4-Methyl-piperazin-1-yl)-pyrimidin-4-yl]-2-(6-methyl-pyridin-2-yl)-imidazo[1,2-a]pyridine;
3-{[3-(2-Amino-pyrimidin-4-yl)-2-(6-methyl-pyridin-2-yl)-imidazo[1,2-a]pyridine-6-carbonyl]-amino}-propionic acid methyl ester;
3-{[3-(2-Amino-pyrimidin-4-yl)-2-(6-methyl-pyridin-2-yl)-imidazo[1,2-a]pyridine-7-carbonyl]-amino}-propionic acid methyl ester;
3-{4-[2-(6-Methyl-pyridin-2-yl)-imidazo[1,2-a]pyridin-3-yl]-pyrimidin-2-ylamino}-phenol;
4-(2-{4-[2-(6-Methyl-pyridin-2-yl)-imidazo[1,2-a]pyridin-3-yl]-pyrimidin-2-ylamino}-ethyl)-benzenesulfonamide;
4-(2-Pyridin-2-yl-imidazo[1,2-a]pyridin-3-yl)-pyrimidin-2-ylamine;
4-[2-(6-Chloro-pyridin-2-yl)-imidazo[1,2-a]pyridin-3-yl]-pyrimidin-2-ylamine;
4-[2-(6-Methyl-pyridin-2-yl)-7-trifluoromethyl-imidazo[1,2-a]pyridin-3-yl]-pyrimidin-2-ylamine;
4-[2-(6-Methyl-pyridin-2-yl)-imidazo[1,2-a]pyridin-3-yl]-pyrimidin-2-ylamine;
4-[2-(6-Methyl-pyridin-2-yl)-imidazo[1,2-a]pyridin-3-yl]-pyrimidine-2-carbonitrile;
4-[2-(6-Methyl-pyridin-2-yl)-imidazo[1,2-a]pyridin-3-yl]-pyrimidine-2-carboxylic acid amide;
4-[6-Bromo-2-(6-methyl-pyridin-2-yl)-imidazo[1,2-a]pyridin-3-yl]-pyrimidin-2-ylamine;
4-[6-Chloro-2-(6-methyl-pyridin-2-yl)-imidazo[1,2-a]pyridin-3-yl]-pyrimidin-2-ylamine;
4-[6-Fluoro-2-(6-methyl-pyridin-2-yl)-imidazo[1,2-a]pyridin-3-yl]-pyrimidin-2-ylamine;
4-[6-Methyl-2-(6-methyl-pyridin-2-yl)-8-(2-morpholin-4-yl-ethylamino)-imidazo[1,2-a]pyridin-3-yl]-pyrimidin-2-ol;
4-[6-Methyl-2-(6-methyl-pyridin-2-yl)-8-(2-pyridin-2-yl-ethylamino)-imidazo[1,2-a]pyridin-3-yl]-pyrimidin-2-ol;
4-[6-Methyl-2-(6-methyl-pyridin-2-yl)-8-(2-pyridin-3-yl-ethylamino)-imidazo[1,2-a]pyridin-3-yl]-pyrimidin-2-ol;
4-[6-Methyl-2-(6-methyl-pyridin-2-yl)-8-(2-pyridin-4-yl-ethylamino)-imidazo[1,2-a]pyridin-3-yl]-pyrimidin-2-ol;
4-[6-Methyl-2-(6-methyl-pyridin-2-yl)-8-morpholin-4-yl-imidazo[1,2-a]pyridin-3-yl]-pyrimidin-2-ol;
4-[6-Methyl-2-(6-methyl-pyridin-2-yl)-8-morpholin-4-yl-imidazo[1,2-a]pyridin-3-yl]-pyrimidin-2-ylamine;
4-[6-Methyl-2-(6-methyl-pyridin-2-yl)-imidazo[1,2-a]pyridin-3-yl]-pyrimidin-2-ylamine;
4-[7-Aminomethyl-2-(6-methyl-pyridin-2-yl)-imidazo[1,2-a]pyridin-3-yl]-pyrimidin-2-ylamine;
4-[7-Methyl-2-(6-methyl-pyridin-2-yl)-imidazo[1,2-a]pyridin-3-yl]-pyrimidin-2-ylamine;
4-[8-Benzyloxy-2-(6-methyl-pyridin-2-yl)-imidazo[1,2-a]pyridin-3-yl]-pyrimidin-2-ol;
4-[8-Benzyloxy-2-(6-methyl-pyridin-2-yl)-imidazo[1,2-a]pyridin-3-yl]-pyrimidin-2-ylamine;
4-[8-Bromo-6-methyl-2-(6-methyl-pyridin-2-yl)-imidazo[1,2-a]pyridin-3-yl]-pyrimidin-2-ol;
4-[8-Methyl-2-(6-methyl-pyridin-2-yl)-imidazo[1,2-a]pyridin-3-yl]-pyrimidin-2-ylamine;
6-Chloro-3-(2-methanesulfonyl-pyrimidin-4-yl)-2-(6-methyl-pyridin-2-yl)-imidazo[1,2-a]pyridine;
5-Dimethylamino-naphthalene-1-sulfonic acid (4-{4-[2-(6-methyl-pyridin-2-yl)-imidazo[1,2-a]pyridin-3-yl]-pyrimidin-2-ylamino}-butyl)-amide;
6-(2,7-Difluoro-6-hydroxy-3-oxo-3H-xanthen-9-yl)-N-(4-{4-[2-(6-methyl-pyridin-2-yl)-imidazo[1,2-a]pyridin-3-yl]-pyrimidin-2-ylamino}-butyl)-isophthalamic acid;
6-Amino-9-[2-carboxy-5-(4-{4-[2-(6-methyl-pyridin-2-yl)-imidazo[1,2-a]pyridin-3-yl]-pyrimidin-2-ylamino}-butylcarbamoyl)-phenyl]-xanthen-3-ylidene-ammonium;
6-Bromo-2-(6-methyl-pyridin-2-yl)-3-(2-methylsulfanyl-pyrimidin-4-yl)-imidazo[1,2-a]pyridine;
6-Fluoro-2-(6-methyl-pyridin-2-yl)-3-(2-methylsulfanyl-pyrimidin-4-yl)-imidazo[1,2-a]pyridine;
7-Amino-4-methyl-3-[(4-{4-[2-(6-methyl-pyridin-2-yl)-imidazo[1,2-a]pyridin-3-yl]-pyrimidin-2-ylamino}-butylcarbamoyl)-methyl]-2-oxo-2H-chromene-6-sulfonic acid;
Cyclobutyl-{4-[2-(6-methyl-pyridin-2-yl)-imidazo[1,2-a]pyridin-3-yl]-pyrimidin-2-yl}-amine;
Cyclopentyl-{4-[2-(6-methyl-pyridin-2-yl)-imidazo[1,2-a]pyridin-3-yl]-pyrimidin-2-yl}-amine;
Cyclopropyl-{4-[2-(6-methyl-pyridin-2-yl)-imidazo[1,2-a]pyridin-3-yl]-pyrimidin-2-yl}-amine;
Cyclopropyl-methyl-{4-[2-(6-methyl-pyridin-2-yl)-imidazo[1,2-a]pyridin-3-yl]-pyrimidin-2-yl}-amine;
Dimethyl-{4-[2-(6-methyl-pyridin-2-yl)-imidazo[1,2-a]pyridin-3-yl]-pyrimidin-2-yl}-amine;
Isopropyl-{4-[2-(6-methyl-pyridin-2-yl)-imidazo[1,2-a]pyridin-3-yl]-pyrimidin-2-yl}-amine;
Methyl-{4-[2-(6-methyl-pyridin-2-yl)-imidazo[1,2-a]pyridin-3-yl]-pyrimidin-2-yl}-amine;
N-(2-{4-[2-(6-Methyl-pyridin-2-yl)-imidazo[1,2-a]pyridin-3-yl]-pyrimidin-2-ylamino}-ethyl)-acetamide;
N-(4-{4-[2-(6-Methyl-pyridin-2-yl)-imidazo[1,2-a]pyridin-3-yl]-pyrimidin-2-ylamino}-butyl)-acetamide;
N,N-Dimethyl-N′-{4-[2-(6-methyl-pyridin-2-yl)-imidazo[1,2-a]pyridin-3-yl]-pyrimidin-2-yl}-ethane-1,2-diamine;
N-[2-(6-Methyl-pyridin-2-yl)-3-(2-methylsulfanyl-pyrimidin-4-yl)-imidazo[1,2-a]pyrimidin-7-yl]-3-pyridin-3-yl-propionamide;
N-[2-(6-Methyl-pyridin-2-yl)-3-(2-methylsulfanyl-pyrimidin-4-yl)-imidazo[1,2-a]pyrimidin-7-yl]-nicotinamide;
N-[2-(6-Methyl-pyridin-2-yl)-3-(2-methylsulfanyl-pyrimidin-4-yl)-imidazo[1,2-a]pyrimidin-7-yl]-propionamide;
N-[3-(2-Amino-pyrimidin-4-yl)-2-(6-methyl-pyridin-2-yl)-imidazo[1,2-a]pyridine-6-carbonyl]-methanesulfonamide;
N-[3-(2-Amino-pyrimidin-4-yl)-2-(6-methyl-pyridin-2-yl)-imidazo[1,2-a]pyridine-7-carbonyl]-methanesulfonamide;
N-[3-(2-Amino-pyrimidin-4-yl)-2-(6-methyl-pyridin-2-yl)-imidazo[1,2-a]pyrimidin-7-yl]-2-(3-methoxy-phenyl)-acetamide;
N-[3-(2-Amino-pyrimidin-4-yl)-2-(6-methyl-pyridin-2-yl)-imidazo[1,2-a]pyrimidin-7-yl]-3,3-dimethyl-butyramide;
N-[3-(2-Amino-pyrimidin-4-yl)-2-(6-methyl-pyridin-2-yl)-imidazo[1,2-a]pyrimidin-7-yl]-3-pyridin-3-yl-propionamide;
N-[3-(2-Amino-pyrimidin-4-yl)-2-(6-methyl-pyridin-2-yl)-imidazo[1,2-a]pyrimidin-7-yl]-acetamide;
N-[3-(2-Amino-pyrimidin-4-yl)-2-(6-methyl-pyridin-2-yl)-imidazo[1,2-a]pyrimidin-7-yl]-nicotinamide;
N-[3-(2-Methanesulfonyl-pyrimidin-4-yl)-2-(6-methyl-pyridin-2-yl)-imidazo[1,2-a]pyrimidin-7-yl]-2-(3-methoxy-phenyl)-acetamide;
N-[3-(2-Methanesulfonyl-pyrimidin-4-yl)-2-(6-methyl-pyridin-2-yl)-imidazo[1,2-a]pyrimidin-7-yl]-3,3-dimethyl-butyramide;
N-[3-(2-Methanesulfonyl-pyrimidin-4-yl)-2-(6-methyl-pyridin-2-yl)-imidazo[1,2-a]pyrimidin-7-yl]-3-pyridin-3-yl-propionamide;
N-[3-(2-Methanesulfonyl-pyrimidin-4-yl)-2-(6-methyl-pyridin-2-yl)-imidazo[1,2-a]pyrimidin-7-yl]-nicotinamide;
N-[3-(2-Methanesulfonyl-pyrimidin-4-yl)-2-(6-methyl-pyridin-2-yl)-imidazo[1,2-a]pyrimidin-7-yl]-propionamide;
N-[3-(2-Amino-pyrimidin-4-yl)-2-(6-methyl-pyridin-2-yl)-imidazo[1,2-a]pyrimidin-7-yl]-propionamide;
N-{4-[2-(6-Methyl-pyridin-2-yl)-imidazo[1,2-a]pyridin-3-yl]-pyrimidin-2-yl}-acetamide;
N1-{4-[2-(6-Methyl-pyridin-2-yl)-imidazo[1,2-a]pyridin-3-yl]-pyrimidin-2-yl}-butane-1,4-diamine;
N1-{4-[2-(6-Methyl-pyridin-2-yl)-imidazo[1,2-a]pyridin-3-yl]-pyrimidin-2-yl}-propane-1,3-diamine;
N-(4-{4-[2-(6-Methyl-pyridin-2-yl)-imidazo[1,2-a]pyridin-3-yl]-pyrimidin-2-ylamino}-butyl)-(BODIPY FL) amide;
N-(4-{4-[2-(6-Methyl-pyridin-2-yl)-imidazo[1,2-a]pyridin-3-yl]-pyrimidin-2-ylamino}-butyl)-(Texas Red-X) amide;
or pharmaceutically acceptable salts or N-oxides thereof.

31. The compound of claim 1, selected from
(2-Methoxy-ethyl)-{4-[2-(6-methyl-pyridin-2-yl)-imidazo[1,2-a]pyridin-3-yl]-pyrimidin-2-yl}-amine;
(3-{4-[2-(6-Methyl-pyridin-2-yl)-imidazo[1,2-a]pyridin-3-yl]-pyrimidin-2-ylamino}-propyl)-carbamic acid tert-butyl ester;
(3-Imidazol-1-yl-propyl)-{4-[2-(6-methyl-pyridin-2-yl)-imidazo[1,2-a]pyridin-3-yl]-pyrimidin-2-yl}-amine;
(4-Amino-benzyl)-{4-[2-(6-methyl-pyridin-2-yl)-imidazo[1,2-a]pyridin-3-yl]-pyrimidin-2-yl}-amine;
(5-{4-[2-(6-Methyl-pyridin-2-yl)-imidazo[1,2-a]pyridin-3-yl]-pyrimidin-2-ylamino}-pentyl)-carbamic acid tert-butyl ester;
[3-(2-Amino-pyrimidin-4-yl)-2-(6-methyl-pyridin-2-yl)-imidazo[1,2-a]pyridin-6-yl]-methanol;
[3-(2-amino-pyrimidin-4-yl)-2-(6-methyl-pyridin-2-yl)-imidazo[1,2-a]pyridin-7-yl]-methanol;
[3-(2-Amino-pyrimidin-4-yl)-6-methyl-2-(6-methyl-pyridin-2-yl)-imidazo[1,2-a]pyridin-8-yl]-(2-pyridin-2-yl-ethyl)-amine;
[3-(4-Methyl-piperazin-1-yl)-propyl]-{4-[2-(6-methyl-pyridin-2-yl)-imidazo[1,2-a]pyridin-3-yl]-pyrimidin-2-yl}-amine;
[3-(4-Methyl-piperidin-1-yl)-propyl]-{4-[2-(6-methyl-pyridin-2-yl)-imidazo[1,2-a]pyridin-3-yl]-pyrimidin-2-yl}-amine;
{4-[2-(6-Methyl-pyridin-2-yl)-imidazo[1,2-a]pyridin-3-yl]-pyrimidin-2-yl}-(2H-pyrazol-3-yl)-amine;
{4-[2-(6-Methyl-pyridin-2-yl)-imidazo[1,2-a]pyridin-3-yl]-pyrimidin-2-yl}-(2-morpholin-4-yl-ethyl)-amine;
{4-[2-(6-Methyl-pyridin-2-yl)-imidazo[1,2-a]pyridin-3-yl]-pyrimidin-2-yl}-(2-pyridin-2-yl-ethyl)-amine;
{4-[2-(6-Methyl-pyridin-2-yl)-imidazo[1,2-a]pyridin-3-yl]-pyrimidin-2-yl}-(2-pyridin-3-yl-ethyl)-amine;
{4-[2-(6-Methyl-pyridin-2-yl)-imidazo[1,2-a]pyridin-3-yl]-pyrimidin-2-yl}-(2-pyridin-4-yl-ethyl)-amine;
{4-[2-(6-Methyl-pyridin-2-yl)-imidazo[1,2-a]pyridin-3-yl]-pyrimidin-2-yl}-(3-morpholin-4-yl-propyl)-amine;
{4-[2-(6-Methyl-pyridin-2-yl)-imidazo[1,2-a]pyridin-3-yl]-pyrimidin-2-yl}-(3-piperidin-1-yl-propyl)-amine;
{4-[2-(6-Methyl-pyridin-2-yl)-imidazo[1,2-a]pyridin-3-yl]-pyrimidin-2-yl}-[1,3,4]thiadiazol-2-yl-amine;
2-(6-Methyl-pyridin-2-yl)-3-pyrimidin-4-yl-imidazo[1,2-a]pyrimidin-7-ylamine;
3-(2-amino-pyrimidin-4-yl)-2-(6-methyl-pyridin-2-yl)-imidazo[1,2-a]pyridine-6-carbonitrile;
3-(2-Amino-pyrimidin-4-yl)-2-(6-methyl-pyridin-2-yl)-imidazo[1,2-a]pyridine-6-carboxylic acid (2-methoxy-ethyl)-amide;
3-(2-amino-pyrimidin-4-yl)-2-(6-methyl-pyridin-2-yl)-imidazo[1,2-a]pyridine-6-carboxylic acid amide;
3-(2-Amino-pyrimidin-4-yl)-2-(6-methyl-pyridin-2-yl)-imidazo[1,2-a]pyridine-6-carboxylic acid cyclopropylamide;
3-(2-Amino-pyrimidin-4-yl)-2-(6-methyl-pyridin-2-yl)-imidazo[1,2-a]pyridine-6-carboxylic acid ethyl amide;
3-(2-Amino-pyrimidin-4-yl)-2-(6-methyl-pyridin-2-yl)-imidazo[1,2-a]pyridine-6-carboxylic acid hydroxyamide;
3-(2-Amino-pyrimidin-4-yl)-2-(6-methyl-pyridin-2-yl)-imidazo[1,2-a]pyridine-6-carboxylic acid methoxy-amide;
3-(2-Amino-pyrimidin-4-yl)-2-(6-methyl-pyridin-2-yl)-imidazo[1,2-a]pyridine-7-carboxylic acid (2-thiophen-2-yl-ethyl)-amide;
3-(2-amino-pyrimidin-4-yl)-2-(6-methyl-pyridin-2-yl)-imidazo[1,2-a]pyridine-7-carboxylic acid amide;
3-(2-Amino-pyrimidin-4-yl)-2-(6-methyl-pyridin-2-yl)-imidazo[1,2-a]pyridine-7-carboxylic acid cyclopropylamide;
3-(2-Amino-pyrimidin-4-yl)-2-(6-methyl-pyridin-2-yl)-imidazo[1,2-a]pyridine-7-carboxylic acid ethyl amide;
3-(2-Amino-pyrimidin-4-yl)-2-(6-methyl-pyridin-2-yl)-imidazo[1,2-a]pyridine-7-carboxylic acid methoxy-amide;
3-(2-amino-pyrimidin-4-yl)-2-(6-methyl-pyridin-2-yl)-imidazo[1,2-a]pyrimidin-7-ylamine;
3-(2-Azetidin-1-yl-pyrimidin-4-yl)-2-(6-methyl-pyridin-2-yl)-imidazo[1,2-a]pyridine;
3-[2-(2-Methyl-aziridin-1-yl)-pyrimidin-4-yl]-2-(6-methyl-pyridin-2-yl)-imidazo[1,2-a]pyridine;
3-{[3-(2-Amino-pyrimidin-4-yl)-2-(6-methyl-pyridin-2-yl)-imidazo[1,2-a]pyridine-7-carbonyl]-amino}-propionic acid methyl ester;
4-(2-{4-[2-(6-Methyl-pyridin-2-yl)-imidazo[1,2-a]pyridin-3-yl]-pyrimidin-2-ylamino}-ethyl)-benzenesulfonamide;
4-[2-(6-Chloro-pyridin-2-yl)-imidazo[1,2-a]pyridin-3-yl]-pyrimidin-2-ylamine;
4-[2-(6-methyl-pyridin-2-yl)-imidazo[1,2-a]pyridin-3-yl]-pyrimidin-2-ylamine;
4-[2-(6-methyl-pyridin-2-yl)-imidazo[1,2-a]pyridin-3-yl]-pyrimidine-2-carbonitrile;
4-[6-Bromo-2-(6-methyl-pyridin-2-yl)-imidazo[1,2-a]pyridin-3-yl]-pyrimidin-2-ylamine;
4-[6-Chloro-2-(6-methyl-pyridin-2-yl)-imidazo[1,2-a]pyridin-3-yl]-pyrimidin-2-ylamine;
4-[6-Fluoro-2-(6-methyl-pyridin-2-yl)-imidazo[1,2-a]pyridin-3-yl]-pyrimidin-2-ylamine;
4-[6-methyl-2-(6-methyl-pyridin-2-yl)-imidazo[1,2-a]pyridin-3-yl]-pyrimidin-2-ylamine;
4-[7-aminomethyl-2-(6-methyl-pyridin-2-yl)-imidazo[1,2-a]pyridin-3-yl]-pyrimidin-2-ylamine;
4-[7-Methyl-2-(6-methyl-pyridin-2-yl)-imidazo[1,2-a]pyridin-3-yl]-pyrimidin-2-ylamine;
4-[8-Methyl-2-(6-methyl-pyridin-2-yl)-imidazo[1,2-a]pyridin-3-yl]-pyrimidin-2-ylamine;
6-(2,7-Difluoro-6-hydroxy-3-oxo-3H-xanthen-9-yl)-N-(4-{4-[2-(6-methyl-pyridin-2-yl)-imidazo[1,2-a]pyridin-3-yl]-pyrimidin-2-ylamino}-butyl)-isophthalamic acid;
7-Amino-4-methyl-3-[(4-{4-[2-(6-methyl-pyridin-2-yl)-imidazo[1,2-a]pyridin-3-yl]-pyrimidin-2-ylamino}-butylcarbamoyl)-methyl]-2-oxo-2H-chromene-6-sulfonic acid;
Cyclobutyl-{4-[2-(6-methyl-pyridin-2-yl)-imidazo[1,2-a]pyridin-3-yl]-pyrimidin-2-yl}-amine;
Cyclopentyl-{4-[2-(6-methyl-pyridin-2-yl)-imidazo[1,2-a]pyridin-3-yl]-pyrimidin-2-yl}-amine;
Cyclopropyl-{4-[2-(6-methyl-pyridin-2-yl)-imidazo[1,2-a]pyridin-3-yl]-pyrimidin-2-yl}-amine;
Cyclopropyl-methyl-{4-[2-(6-methyl-pyridin-2-yl)-imidazo[1,2-a]pyridin-3-yl]-pyrimidin-2-yl}-amine;
Dimethyl-{4-[2-(6-methyl-pyridin-2-yl)-imidazo[1,2-a]pyridin-3-yl]-pyrimidin-2-yl}-amine;
Isopropyl-{4-[2-(6-methyl-pyridin-2-yl)-imidazo[1,2-a]pyridin-3-yl]-pyrimidin-2-yl}-amine;
Methyl-{4-[2-(6-methyl-pyridin-2-yl)-imidazo[1,2-a]pyridin-3-yl]-pyrimidin-2-yl}-amine;
N-(2-{4-[2-(6-Methyl-pyridin-2-yl)-imidazo[1,2-a]pyridin-3-yl]-pyrimidin-2-ylamino}-ethyl)-acetamide;
N-(4-{4-[2-(6-Methyl-pyridin-2-yl)-imidazo[1,2-a]pyridin-3-yl]-pyrimidin-2-ylamino}-butyl)-acetamide;
N-[3-(2-amino-pyrimidin-4-yl)-2-(6-methyl-pyridin-2-yl)-imidazo[1,2-a]pyrimidin-7-yl]-2-(3-methoxy-phenyl)-acetamide;
N-[3-(2-Amino-pyrimidin-4-yl)-2-(6-methyl-pyridin-2-yl)-imidazo[1,2-a]pyrimidin-7-yl]-3,3-dimethyl-butyramide;
N-[3-(2-amino-pyrimidin-4-yl)-2-(6-methyl-pyridin-2-yl)-imidazo[1,2-a]pyrimidin-7-yl]-3-pyridin-3-yl-propionamide;
N-[3-(2-amino-pyrimidin-4-yl)-2-(6-methyl-pyridin-2-yl)-imidazo[1,2-a]pyrimidin-7-yl]-acetamide;
N-[3-(2-amino-pyrimidin-4-yl)-2-(6-methyl-pyridin-2-yl)-imidazo[1,2-a]pyrimidin-7-yl]-nicotinamide;
N-[3-(2-amino-pyrimidin-4-yl)-2-(6-methyl-pyridin-2-yl)-imidazo[1,2-a]pyrimidin-7-yl]-propionamide;
N-{4-[2-(6-methyl-pyridin-2-yl)-imidazo[1,2-a]pyridin-3-yl]-pyrimidin-2-yl}-acetamide;
N1-{4-[2-(6-Methyl-pyridin-2-yl)-imidazo[1,2-a]pyridin-3-yl]-pyrimidin-2-yl}-butane-1,4-diamine;
(4-{4-[2-(6-Methyl-pyridin-2-yl)-imidazo[1,2-a]pyridin-3-yl]-pyrimidin-2-ylamino}-butyl)-carbamic acid tert-butyl ester;
[3-(2-Amino-pyrimidin-4-yl)-6-methyl-2-(6-methyl-pyridin-2-yl)-imidazo[1,2-a]pyridin-8-yl]-(2-morpholin-4-yl-ethyl)-amine;
[3-(2-Amino-pyrimidin-4-yl)-6-methyl-2-(6-methyl-pyridin-2-yl)-imidazo[1,2-a]pyridin-8-yl]-(2-pyridin-3-yl-ethyl)-amine;
[3-(2-Amino-pyrimidin-4-yl)-6-methyl-2-(6-methyl-pyridin-2-yl)-imidazo[1,2-a]pyridin-8-yl]-(2-pyridin-4-yl-ethyl)-amine;
[3-(2-Amino-pyrimidin-4-yl)-6-methyl-2-(6-methyl-pyridin-2-yl)-imidazo[1,2-a]pyridin-8-yl]-(3-morpholin-4-yl-propyl)-amine;
[4-(2-Pyridin-2-yl-imidazo[1,2-a]pyridin-3-yl)-pyrimidin-2-yl]-pyridin-3-ylmethyl-amine;
{4-[2-(6-Methyl-pyridin-2-yl)-imidazo[1,2-a]pyridin-3-yl]-pyrimidin-2-yl}-((R)-1-phenyl-ethyl)-amine;
{4-[2-(6-Methyl-pyridin-2-yl)-imidazo[1,2-a]pyridin-3-yl]-pyrimidin-2-yl}-((S)-1-phenyl-ethyl)-amine;
{4-[2-(6-methyl-pyridin-2-yl)-imidazo[1,2-a]pyridin-3-yl]-pyrimidin-2-yl}-(1H-tetrazol-5-yl)-amine;
{7,7-Dimethyl-8-[5-(4-{4-[2-(6-methyl-pyridin-2-yl)-imidazo[1,2-a]pyridin-3-yl]-pyrimidin-2-ylamino}-butylcarbamoyl)-pentyl]-2-oxo-4-trifluoromethyl-7,8-dihydro-2H-1-oxa-8-aza-anthracen-5-yl}-methanesulfonic acid;
2-(6-Methyl-pyridin-2-yl)-3-(2-morpholin-4-yl-pyrimidin-4-yl)-imidazo[1,2-a]pyridine;
2-(6-Methyl-pyridin-2-yl)-3-(2-piperidin-1-yl-pyrimidin-4-yl)-imidazo[1,2-a]pyridine;
2-(6-Methyl-pyridin-2-yl)-3-(2-pyrrolidin-1-yl-pyrimidin-4-yl)-imidazo[1,2-a]pyridine;
2-(6-Methyl-pyridin-2-yl)-3-pyrimidin-4-yl-imidazo[1,2-a]pyridine;
3-(2-Amino-pyrimidin-4-yl)-2-(6-methyl-pyridin-2-yl)-imidazo[1,2-a]pyridin-8-ylamine;
3-(2-Amino-pyrimidin-4-yl)-2-(6-methyl-pyridin-2-yl)-imidazo[1,2-a]pyridine-6-carboxylic acid;
3-(2-Amino-pyrimidin-4-yl)-2-(6-methyl-pyridin-2-yl)-imidazo[1,2-a]pyridine-6-carboxylic acid ([1,4]dioxan-2-ylmethyl)-amide;
3-(2-Amino-pyrimidin-4-yl)-2-(6-methyl-pyridin-2-yl)-imidazo[1,2-a]pyridine-6-carboxylic acid ([1,4]dioxan-2-ylmethyl)-amide;
3-(2-Amino-pyrimidin-4-yl)-2-(6-methyl-pyridin-2-yl)-imidazo[1,2-a]pyridine-6-carboxylic acid (2-dimethylamino-ethyl)-amide;
3-(2-Amino-pyrimidin-4-yl)-2-(6-methyl-pyridin-2-yl)-imidazo[1,2-a]pyridine-6-carboxylic acid (2-thiophen-2-yl-ethyl)-amide;
3-(2-Amino-pyrimidin-4-yl)-2-(6-methyl-pyridin-2-yl)-imidazo[1,2-a]pyridine-6-carboxylic acid methyl ester;
3-(2-Amino-pyrimidin-4-yl)-2-(6-methyl-pyridin-2-yl)-imidazo[1,2-a]pyridine-7-carboxylic acid ([1,4]dioxan-2-ylmethyl)-amide;
3-(2-Amino-pyrimidin-4-yl)-2-(6-methyl-pyridin-2-yl)-imidazo[1,2-a]pyridine-7-carboxylic acid (2-dimethylamino-ethyl)-amide;
3-(2-Amino-pyrimidin-4-yl)-2-(6-methyl-pyridin-2-yl)-imidazo[1,2-a]pyridine-7-carboxylic acid (2-hydroxy-ethyl)-amide;
3-(2-Amino-pyrimidin-4-yl)-2-(6-methyl-pyridin-2-yl)-imidazo[1,2-a]pyridine-7-carboxylic acid (2-oxo-2-pyridin-3-yl-ethyl)-amide;
3-(2-Amino-pyrimidin-4-yl)-2-(6-methyl-pyridin-2-yl)-imidazo[1,2-a]pyridine-7-carboxylic acid (piperidin-3-ylmethyl)-amide;
3-(2-Amino-pyrimidin-4-yl)-2-(6-methyl-pyridin-2-yl)-imidazo[1,2-a]pyridine-7-carboxylic acid ethyl ester;
3-(2-Methylsulfanyl-pyrimidin-4-yl)-2-pyridin-2-yl-imidazo[1,2-a]pyridine;
3-[2-(4-Methyl-piperazin-1-yl)-pyrimidin-4-yl]-2-(6-methyl-pyridin-2-yl)-imidazo[1,2-a]pyridine;
3-{[3-(2-Amino-pyrimidin-4-yl)-2-(6-methyl-pyridin-2-yl)-imidazo[1,2-a]pyridine-6-carbonyl]-amino}-propionic acid methyl ester;
4-(2-Pyridin-2-yl-imidazo[1,2-a]pyridin-3-yl)-pyrimidin-2-ylamine;
4-[2-(6-Methyl-pyridin-2-yl)-7-trifluoromethyl-imidazo[1,2-a]pyridin-3-yl]-pyrimidin-2-ylamine;
4-[2-(6-Methyl-pyridin-2-yl)-imidazo[1,2-a]pyridin-3-yl]-pyrimidine-2-carboxylic acid amide;
4-[6-Methyl-2-(6-methyl-pyridin-2-yl)-8-(2-morpholin-4-yl-ethylamino)-imidazo[1,2-a]pyridin-3-yl]-pyrimidin-2-ol;
4-[6-Methyl-2-(6-methyl-pyridin-2-yl)-8-morpholin-4-yl-imidazo[1,2-a]pyridin-3-yl]-pyrimidin-2-ylamine;
4-[8-Benzyloxy-2-(6-methyl-pyridin-2-yl)-imidazo[1,2-a]pyridin-3-yl]-pyrimidin-2-ol;
4-[8-Benzyloxy-2-(6-methyl-pyridin-2-yl)-imidazo[1,2-a]pyridin-3-yl]-pyrimidin-2-ylamine;
4-[8-Bromo-6-methyl-2-(6-methyl-pyridin-2-yl)-imidazo[1,2-a]pyridin-3-yl]-pyrimidin-2-ol;
5-Dimethylamino-naphthalene-1-sulfonic acid (4-{4-[2-(6-methyl-pyridin-2-yl)-imidazo[1,2-a]pyridin-3-yl]-pyrimidin-2-ylamino}-butyl)-amide;
6-Amino-9-[2-carboxy-5-(4-{4-[2-(6-methyl-pyridin-2-yl)-imidazo[1,2-a]pyridin-3-yl]-pyrimidin-2-ylamino}-butylcarbamoyl)-phenyl]-xanthen-3-ylidene-ammonium;
6-Bromo-2-(6-methyl-pyridin-2-yl)-3-(2-methylsulfanyl-pyrimidin-4-yl)-imidazo[1,2-a]pyridine;
6-Fluoro-2-(6-methyl-pyridin-2-yl)-3-(2-methylsulfanyl-pyrimidin-4-yl)-imidazo[1,2-a]pyridine;
N,N-Dimethyl-N′-{4-[2-(6-methyl-pyridin-2-yl)-imidazo[1,2-a]pyridin-3-yl]-pyrimidin-2-yl}-ethane-1,2-diamine;
N1-{4-[2-(6-Methyl-pyridin-2-yl)-imidazo[1,2-a]pyridin-3-yl]-pyrimidin-2-yl}-propane-1,3-diamine;
N-(4-{4-[2-(6-Methyl-pyridin-2-yl)-imidazo[1,2-a]pyridin-3-yl]-pyrimidin-2-ylamino}-butyl)-(BODIPY FL) amide;
N-(4-{4-[2-(6-Methyl-pyridin-2-yl)-imidazo[1,2-a]pyridin-3-yl]-pyrimidin-2-ylamino}-butyl)-(Texas Red-X) amide;
or pharmaceutically acceptable salts or N-oxides thereof.

32. The compound of claim 1, selected from
(2-Methoxy-ethyl)-{4-[2-(6-methyl-pyridin-2-yl)-imidazo[1,2-a]pyridin-3-yl]-pyrimidin-2-yl}-amine;
(3-{4-[2-(6-Methyl-pyridin-2-yl)-imidazo[1,2-a]pyridin-3-yl]-pyrimidin-2-ylamino}-propyl)-carbamic acid tert-butyl ester;
(3-Imidazol-1-yl-propyl)-{4-[2-(6-methyl-pyridin-2-yl)-imidazo[1,2-a]pyridin-3-yl]-pyrimidin-2-yl}-amine;
(4-Amino-benzyl)-{4-[2-(6-methyl-pyridin-2-yl)-imidazo[1,2-a]pyridin-3-yl]-pyrimidin-2-yl}-amine;
(5-{4-[2-(6-Methyl-pyridin-2-yl)-imidazo[1,2-a]pyridin-3-yl]-pyrimidin-2-ylamino}-pentyl)-carbamic acid tert-butyl ester;
[3-(2-Amino-pyrimidin-4-yl)-2-(6-methyl-pyridin-2-yl)-imidazo[1,2-a]pyridin-6-yl]-methanol;
[3-(2-amino-pyrimidin-4-yl)-2-(6-methyl-pyridin-2-yl)-imidazo[1,2-a]pyridin-7-yl]-methanol;
[3-(2-Amino-pyrimidin-4-yl)-6-methyl-2-(6-methyl-pyridin-2-yl)-imidazo[1,2-a]pyridin-8-yl]-(2-pyridin-2-yl-ethyl)-amine;
[3-(4-Methyl-piperazin-1-yl)-propyl]-{4-[2-(6-methyl-pyridin-2-yl)-imidazo[1,2-a]pyridin-3-yl]-pyrimidin-2-yl}-amine;
[3-(4-Methyl-piperidin-1-yl)-propyl]-{4-[2-(6-methyl-pyridin-2-yl)-imidazo[1,2-a]pyridin-3-yl]-pyrimidin-2-yl}-amine;
{4-[2-(6-Methyl-pyridin-2-yl)-imidazo[1,2-a]pyridin-3-yl]-pyrimidin-2-yl}-(2H-pyrazol-3-yl)-amine;
{4-[2-(6-Methyl-pyridin-2-yl)-imidazo[1,2-a]pyridin-3-yl]-pyrimidin-2-yl}-(2-morpholin-4-yl-ethyl)-amine;
{4-[2-(6-Methyl-pyridin-2-yl)-imidazo[1,2-a]pyridin-3-yl]-pyrimidin-2-yl}-(2-pyridin-2-yl-ethyl)-amine;
{4-[2-(6-Methyl-pyridin-2-yl)-imidazo[1,2-a]pyridin-3-yl]-pyrimidin-2-yl}-(2-pyridin-3-yl-ethyl)-amine;
{4-[2-(6-Methyl-pyridin-2-yl)-imidazo[1,2-a]pyridin-3-yl]-pyrimidin-2-yl}-(2-pyridin-4-yl-ethyl)-amine;
{4-[2-(6-Methyl-pyridin-2-yl)-imidazo[1,2-a]pyridin-3-yl]-pyrimidin-2-yl}-(3-morpholin-4-yl-propyl)-amine;
{4-[2-(6-Methyl-pyridin-2-yl)-imidazo[1,2-a]pyridin-3-yl]-pyrimidin-2-yl}-(3-piperidin-1-yl-propyl)-amine;
{4-[2-(6-Methyl-pyridin-2-yl)-imidazo[1,2-a]pyridin-3-yl]-pyrimidin-2-yl}-[1,3,4]thiadiazol-2-yl-amine;
2-(6-Methyl-pyridin-2-yl)-3-pyrimidin-4-yl-imidazo[1,2-a]pyrimidin-7-ylamine;
3-(2-amino-pyrimidin-4-yl)-2-(6-methyl-pyridin-2-yl)-imidazo[1,2-a]pyridine-6-carbonitrile;
3-(2-Amino-pyrimidin-4-yl)-2-(6-methyl-pyridin-2-yl)-imidazo[1,2-a]pyridine-6-carboxylic acid (2-methoxy-ethyl)-amide;
3-(2-amino-pyrimidin-4-yl)-2-(6-methyl-pyridin-2-yl)-imidazo[1,2-a]pyridine-6-carboxylic acid amide;
3-(2-Amino-pyrimidin-4-yl)-2-(6-methyl-pyridin-2-yl)-imidazo[1,2-a]pyridine-6-carboxylic acid cyclopropylamide;
3-(2-Amino-pyrimidin-4-yl)-2-(6-methyl-pyridin-2-yl)-imidazo[1,2-a]pyridine-6-carboxylic acid ethylamide;
3-(2-Amino-pyrimidin-4-yl)-2-(6-methyl-pyridin-2-yl)-imidazo[1,2-a]pyridine-6-carboxylic acid hydroxyamide;
3-(2-Amino-pyrimidin-4-yl)-2-(6-methyl-pyridin-2-yl)-imidazo[1,2-a]pyridine-6-carboxylic acid methoxy-amide;
3-(2-Amino-pyrimidin-4-yl)-2-(6-methyl-pyridin-2-yl)-imidazo[1,2-a]pyridine-7-carboxylic acid (2-thiophen-2-yl-ethyl)-amide;
3-(2-amino-pyrimidin-4-yl)-2-(6-methyl-pyridin-2-yl)-imidazo[1,2-a]pyridine-7-carboxylic acid amide;
3-(2-Amino-pyrimidin-4-yl)-2-(6-methyl-pyridin-2-yl)-imidazo[1,2-a]pyridine-7-carboxylic acid cyclopropylamide;
3-(2-Amino-pyrimidin-4-yl)-2-(6-methyl-pyridin-2-yl)-imidazo[1,2-a]pyridine-7-carboxylic acid ethyl amide;
3-(2-Amino-pyrimidin-4-yl)-2-(6-methyl-pyridin-2-yl)-imidazo[1,2-a]pyridine-7-carboxylic acid methoxy-amide;
3-(2-amino-pyrimidin-4-yl)-2-(6-methyl-pyridin-2-yl)-imidazo[1,2-a]pyrimidin-7-ylamine;
3-(2-Azetidin-1-yl-pyrimidin-4-yl)-2-(6-methyl-pyridin-2-yl)-imidazo[1,2-a]pyridine;
3-[2-(2-Methyl-aziridin-1-yl)-pyrimidin-4-yl]-2-(6-methyl-pyridin-2-yl)-imidazo[1,2-a]pyridine;
3-{[3-(2-Amino-pyrimidin-4-yl)-2-(6-methyl-pyridin-2-yl)-imidazo[1,2-a]pyridine-7-carbonyl]-amino}-propionic acid methyl ester;
4-(2-{4-[2-(6-Methyl-pyridin-2-yl)-imidazo[1,2-a]pyridin-3-yl]-pyrimidin-2-ylamino}-ethyl)-benzenesulfonamide;
4-[2-(6-Chloro-pyridin-2-yl)-imidazo[1,2-a]pyridin-3-yl]-pyrimidin-2-ylamine;
4-[2-(6-methyl-pyridin-2-yl)-imidazo[1,2-a]pyridin-3-yl]-pyrimidin-2-ylamine;
4-[2-(6-methyl-pyridin-2-yl)-imidazo[1,2-a]pyridin-3-yl]-pyrimidine-2-carbonitrile;
4-[6-Bromo-2-(6-methyl-pyridin-2-yl)-imidazo[1,2-a]pyridin-3-yl]-pyrimidin-2-ylamine;
4-[6-Chloro-2-(6-methyl-pyridin-2-yl)-imidazo[1,2-a]pyridin-3-yl]-pyrimidin-2-ylamine;
4-[6-Fluoro-2-(6-methyl-pyridin-2-yl)-imidazo[1,2-a]pyridin-3-yl]-pyrimidin-2-ylamine;
4-[6-methyl-2-(6-methyl-pyridin-2-yl)-imidazo[1,2-a]pyridin-3-yl]-pyrimidin-2-ylamine;
4-[7-aminomethyl-2-(6-methyl-pyridin-2-yl)-imidazo[1,2-a]pyridin-3-yl]-pyrimidin-2-ylamine;
4-[7-Methyl-2-(6-methyl-pyridin-2-yl)-imidazo[1,2-a]pyridin-3-yl]-pyrimidin-2-ylamine;
4-[8-Methyl-2-(6-methyl-pyridin-2-yl)-imidazo[1,2-a]pyridin-3-yl]-pyrimidin-2-ylamine;
6-(2,7-Difluoro-6-hydroxy-3-oxo-3H-xanthen-9-yl)-N-(4-{4-[2-(6-methyl-pyridin-2-yl)-imidazo[1,2-a]pyridin-3-yl]-pyrimidin-2-ylamino}-butyl)-isophthalamic acid;
7-Amino-4-methyl-3-[(4-{4-[2-(6-methyl-pyridin-2-yl)-imidazo[1,2-a]pyridin-3-yl]-pyrimidin-2-ylamino}-butylcarbamoyl)-methyl]-2-oxo-2H-chromene-6-sulfonic acid;
Cyclobutyl-{4-[2-(6-methyl-pyridin-2-yl)-imidazo[1,2-a]pyridin-3-yl]-pyrimidin-2-yl}-amine;
Cyclopentyl-{4-[2-(6-methyl-pyridin-2-yl)-imidazo[1,2-a]pyridin-3-yl]-pyrimidin-2-yl}-amine;
Cyclopropyl-{4-[2-(6-methyl-pyridin-2-yl)-imidazo[1,2-a]pyridin-3-yl]-pyrimidin-2-yl}-amine;
Cyclopropyl-methyl-{4-[2-(6-methyl-pyridin-2-yl)-imidazo[1,2-a]pyridin-3-yl]-pyrimidin-2-yl}-amine;
Dimethyl-{4-[2-(6-methyl-pyridin-2-yl)-imidazo[1,2-a]pyridin-3-yl]-pyrimidin-2-yl}-amine;
Isopropyl-{4-[2-(6-methyl-pyridin-2-yl)-imidazo[1,2-a]pyridin-3-yl]-pyrimidin-2-yl}-amine;
Methyl-{4-[2-(6-methyl-pyridin-2-yl)-imidazo[1,2-a]pyridin-3-yl]-pyrimidin-2-yl}-amine;
N-(2-{4-[2-(6-Methyl-pyridin-2-yl)-imidazo[1,2-a]pyridin-3-yl]-pyrimidin-2-ylamino}-ethyl)-acetamide;
N-(4-{4-[2-(6-Methyl-pyridin-2-yl)-imidazo[1,2-a]pyridin-3-yl]-pyrimidin-2-ylamino}-butyl)-acetamide;
N-[3-(2-amino-pyrimidin-4-yl)-2-(6-methyl-pyridin-2-yl)-imidazo[1,2-a]pyrimidin-7-yl]-2-(3-methoxy-phenyl)-acetamide;
N-[3-(2-Amino-pyrimidin-4-yl)-2-(6-methyl-pyridin-2-yl)-imidazo[1,2-a]pyrimidin-7-yl]-3,3-dimethyl-butyramide;
N-[3-(2-amino-pyrimidin-4-yl)-2-(6-methyl-pyridin-2-yl)-imidazo[1,2-a]pyrimidin-7-yl]-3-pyridin-3-yl-propionamide;
N-[3-(2-amino-pyrimidin-4-yl)-2-(6-methyl-pyridin-2-yl)-imidazo[1,2-a]pyrimidin-7-yl]-acetamide;
N-[3-(2-amino-pyrimidin-4-yl)-2-(6-methyl-pyridin-2-yl)-imidazo[1,2-a]pyrimidin-7-yl]-nicotinamide;
N-[3-(2-amino-pyrimidin-4-yl)-2-(6-methyl-pyridin-2-yl)-imidazo[1,2-a]pyrimidin-7-yl]-propionamide;
N-{4-[2-(6-methyl-pyridin-2-yl)-imidazo[1,2-a]pyridin-3-yl]-pyrimidin-2-yl}-acetamide;
N1-{4-[2-(6-Methyl-pyridin-2-yl)-imidazo[1,2-a]pyridin-3-yl]-pyrimidin-2-yl}-butane-1,4-diamine;
or pharmaceutically acceptable salts or N-oxides thereof.

33. A pharmaceutical composition comprising at least one compound of claim 1 and a pharmaceutically acceptable carrier.

34. A pharmaceutical composition comprising at least one compound of claim 30 and a pharmaceutically acceptable carrier.

35. A method of inhibiting the TGFβ signaling pathway in a subject, the method comprising administering to said subject an effective amount of at least one compound of claim 1.

36. A method of inhibiting the TGFβ signaling pathway in a subject, the method comprising administering to said subject an effective amount of at least one compound of claim 30.

37. A method of inhibiting the TGFβ type I receptor in a cell, the method comprising contacting said cell with an effective amount of at least one compound of claim 1.

38. A method of inhibiting the TGFβ type I receptor in a cell, the method comprising contacting said cell with an effective amount of at least one compound of claim 30.

39. A method of reducing the accumulation of excess extracellular matrix induced by TGFβ in a subject, the method comprising administering to said subject an effective amount of at least one compound of claim 1.

40. A method of reducing the accumulation of excess extracellular matrix induced by TGFβ in a subject, the method comprising administering to said subject an effective amount of at least one compound of claim 30.

41. A method of treating or preventing a fibrotic condition in a subject, the method comprising administering to said subject an effective amount of at least one compound of claim 1.

42. A method of treating or preventing a fibrotic condition in a subject, the method comprising administering to said subject an effective amount of at least one compound of claim 30.

43. The method of claim 41 or 42, wherein the fibrotic condition is selected from scleroderma, lupus nephritis, connective tissue disease, wound healing, surgical scarring, spinal cord injury, CNS scarring, acute lung injury, idiopathic pulmonary fibrosis, chronic obstructive pulmonary disease, adult respiratory distress syndrome, acute lung injury, drug-induced lung injury, glomerulonephritis, diabetic nephropathy, hypertension-induced nephropathy, hepatic or biliary fibrosis, liver cirrhosis, renal fibrosis, primary biliary cirrhosis, fatty liver disease, primary sclerosing cholangitis, restenosis, cardiac fibrosis, opthalmic scarring, fibrosclerosis, fibrotic cancers, fibroids, fibroma, fibroadenomas, fibrosarcomas, transplant arteriopathy, radiation therapy-induced fibrosis, chemotherapy-induced fibrosis, and keloid.

44. A method of inhibiting metastasis of tumor cells in a subject, the method comprising administering to said subject an effective amount of at least one compound of claim 1.

45. A method of inhibiting metastasis of tumor cells in a subject, the method comprising administering to said subject an effective amount of at least one compound of claim 30.

46. A method of treating a disease or disorder mediated by an overexpression of TGFβ, the method comprising administering to a subject in need of such treatment an effective amount of at least one compound of claim 1.

47. A method of treating a disease or disorder mediated by an overexpression of TGFβ, the method comprising administering to a subject in need of such treatment an effective amount of at least one compound of claim 30.

48. The method of claim 46 or 47, said disease or disorder being selected from the group consisting of demyelination of neurons in multiple sclerosis, Alzheimer's disease, cerebral angiopathy, squamous cell carcinomas, multiple myeloma, melanoma, glioma, glioblastomas, leukemia, and carcinomas of the lung, breast, ovary, cervix, liver, biliary tract, gastrointestinal tract, pancreas, prostate, and head and neck.
